SEQUENCE LISTING (2 sequences)

SEQ ID NO: 1 - protein, 9 aa, synthetic construct
  REGION 1..9: note = Non-natural peptide substrate of PIM1, S6K/Rsk2peptide
KKRNRTLTK

SEQ ID NO: 2 - protein, 11 aa, synthetic construct
  REGION 1..11: note = Non-natural peptide substrate of PIM2, Pim2tide
RSRHSSYPAGT

(19) United States
(12) Patent Application Publication
(10) Pub. No.: US 2024/0360135 A1
(43) Pub. Date: Oct. 31, 2024

(54) IMIDAZO[1,2-B]PYRIDAZINYL COMPOUNDS AND USES THEREOF

(71) Applicant: Sumitomo Pharma Oncology, Inc., Marlborough, MA (US)

(72) Inventors: Adam Siddiqui-Jain, South Jordan, UT (US); Gaelle L. Mercenne, Millcreek, UT (US); Jason Marc Foulks, Sandy, UT (US); Steven L. Warner, Draper, UT (US)

(21) Appl. No.: 18/293,234

(22) PCT Filed: Jul. 29, 2022
(86) PCT No.: PCT/US2022/074288

§ 371 Date: Jan. 29, 2024

Related U.S. Application Data
(60) Provisional application No. 63/227,579, filed on Jul. 30, 2021.

(51) Int. Cl. C07D 487/04; A61K 31/5025; A61K 45/06

(52) U.S. Cl. CPC C07D 487/04; A61K 31/5025; A61K 45/06

(57) ABSTRACT

Provided herein are compounds of the following structural formula: or a pharmaceutically acceptable salt thereof, wherein values for the variables (e.g., R¹, R² n) are as described herein. Compounds of Structural Formula I and pharmaceutically acceptable salts thereof, pharmaceutical compositions of either of the foregoing, and pharmaceutical combinations of any of the foregoing can be used, e.g., to treat a disease, disorder or condition described herein, such as cancer, fibrosis or inflammation.

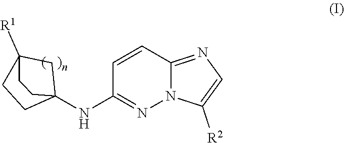

### RELATED APPLICATION

This application claims the benefit of U.S. Provisional Application No. 63/227,579, filed on Jul. 30, 2021. The entire teachings of this application are incorporated herein by reference.

### INCORPORATION BY REFERENCE OF MATERIAL IN XML FILE

This application incorporates by reference the Sequence Listing contained in the following XML file format being submitted via the USPTO patent electronic filing system:
File name: 58513014001_SequenceListing.xml; created Jul. 28, 2022, 4,000 Bytes in size.

### BACKGROUND

Proviral integration site for Moloney murine leukemia virus (PIM) kinases (e.g., PIM1 kinase, PIM2 kinase, PIM3 kinase) are a family of serine/threonine kinases. PIM1 kinase is known to be involved in a number of cytokine signaling pathways as a downstream effector. Once activated, PIM1 kinase causes progression of the cell cycle, inhibition of apoptosis and modulation of other signal transduction pathways, including its own. PIM1 kinase is also known to activate certain transcription factors, such as NFAT, p100, c-Myb and Pap-1, and inhibit others, such as HP1. Normal expression of PIM1 kinase is seen in cells of hematopoietic origin, such as fetal liver, thymus, spleen and bone marrow, as well as prostate and oral epithelial cells.

PIM kinases also play a role in immune regulation. For example, enhanced PIM expression has been observed in a variety of inflammatory states. PIM2 is also implicated in cytokine-induced T-cell growth and survival. One publication (Jackson et al., Cell Immunology, 2012, 272, 200-213) demonstrated in vivo efficacy for a dual PIM1 and PIM3 inhibitor in a mouse inflammatory bowel disease model. Therefore, PIM kinases are attractive targets for various autoimmune and/or inflammatory diseases.

In view of the involvement of PIM kinases in a number of human pathologies, there is a need for specific and selective inhibitors of one or more PIM kinases.

### SUMMARY

Provided herein are compounds that decrease the level of a PIM kinase, pharmaceutically acceptable salts thereof, pharmaceutical compositions of either of the foregoing, and pharmaceutical combinations of any of the foregoing. The compounds described herein can be used in various methods of treatment, e.g., by administering to a subject in need thereof a therapeutically effective amount of the compound, or a pharmaceutically acceptable salt thereof, or a pharmaceutical composition or combination comprising the compound, or a pharmaceutically acceptable salt thereof.

One aspect is a compound of Structural Formula I.

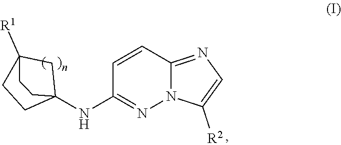

or a pharmaceutically acceptable salt thereof, wherein values for the variables (e.g., R¹, R², n) are as described herein.

Another aspect is a pharmaceutical composition comprising a compound of the present disclosure (e.g., a compound of Structural Formula I, or a subformula thereof, or a pharmaceutically acceptable salt of the foregoing), and one or more pharmaceutically acceptable carriers.

Yet another aspect is a pharmaceutical combination comprising a compound of the present disclosure (e.g., a compound of Structural Formula I, or a subformula thereof, or a pharmaceutically acceptable salt of the foregoing), and one or more additional therapeutic agents.

Another aspect is a method of inhibiting a proviral integration site for Moloney murine leukemia virus (PIM) kinase in a cell, comprising contacting the cell with a compound of the present disclosure (e.g., a compound of Structural Formula I, or a subformula thereof, or a pharmaceutically acceptable salt of the foregoing), for example, a therapeutically effective amount of a compound of the present disclosure.

Another aspect is a method of inhibiting a PIM kinase in a subject, comprising administering to the subject a compound of the present disclosure (e.g., a compound of Structural Formula I, or a subformula thereof, or a pharmaceutically acceptable salt of the foregoing), in an amount effective to inhibit the PIM kinase in the subject, for example, a therapeutically effective amount.

Another aspect is a method of treating a disease, disorder or condition associated with a PIM kinase in a subject in need thereof, comprising administering to the subject a compound of the present disclosure (e.g., a compound of Structural Formula I, or a subformula thereof, or a pharmaceutically acceptable salt of the foregoing), for example, a therapeutically effective amount of a compound of the present disclosure.

Another aspect is a method of treating a fibrotic disease, disorder or condition in a subject in need thereof, comprising administering to the subject a compound of the present disclosure (e.g., a compound of Structural Formula I, or a subformula thereof, or a pharmaceutically acceptable salt of the foregoing), for example, a therapeutically effective amount of a compound of the present disclosure, wherein the fibrotic disease, disorder or condition is present in the absence of a fibrotic cancer.

Another aspect is a method of treating inflammation in a subject in need thereof, comprising administering to the subject a compound of the present disclosure (e.g., a compound of Structural Formula I, or a subformula thereof, or a pharmaceutically acceptable salt of the foregoing), for example, a therapeutically effective amount of a compound of the present disclosure.

Another aspect is a method of treating an inflammatory disease, disorder or condition in a subject in need thereof, comprising administering to the subject a compound of the present disclosure (e.g., a compound of Structural Formula I, or a subformula thereof, or a pharmaceutically acceptable salt of the foregoing), for example, a therapeutically effective amount of a compound of the present disclosure.

Another aspect is a compound of the present disclosure (e.g., a compound of Structural Formula I, or a subformula thereof, or a pharmaceutically acceptable salt of the foregoing), or a composition described herein for use in treating a disorder, disease or condition described herein in a subject. Another aspect is use of a compound of the present disclosure (e.g., a compound of Structural Formula I, or a subformula thereof, or a pharmaceutically acceptable salt of the foregoing) for the manufacture of a medicament for treating a disorder, disease or condition described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments.

FIG. 1 is a representative image of In-Cell Western blot. Top panel shows the expression of total S6 protein detected at 800 nm in red, middle panel shows the expression of phospho-S6 (Ser235/236) measured at 700 nm in geren, and the lower panel shows the overlay of the two signals.

FIG. 2 shows the ratio between phospho-S6 (Ser235/236) and total S6 extracted from the In-Cell Western experiment. Dose response and IC₅₀ were calculated using GraphPad Prism; for Compound 2 and Compound 4, n=3, 2 technical replicates, respectively.

FIG. 3 shows Bad expression in A549 cells in presence of Compound 2 or Compound 4. Top panel shows to total Bad, middle panel shows phosphorylated Bad on residue 112, and bottom panel shows the ratio between phospho-Bad (Ser112) and total Bad. Expression of each protein was normalized to tubulin. Dose response and IC₅₀ were calculated using GraphPad Prism; for Compound 2 and Compound 4, n=3, 2 technical replicates, respectively.

FIG. 4 shows apoptosis measured by quantifying Caspase 3/7 activities in presence of Compound 2 or Compound 4 in A549 cells. Dose response and EC₅₀ were calculated using GraphPad Prism; for Compound 2 and Compound 4, n=3, 2 technical replicates, respectively.

FIG. 5 shows representative images of wound closure in scratched cells in a 96-well ImageLock plate.

FIG. 6 shows time course and percentage of relative wound density quantitatively calculated by IncuCyte software.

FIG. 7 shows the protocol used for the bleomycin-induced lung fibrosis model.

FIG. 8 shows the amount of hydroxyproline (micrograms hydroxyproline per milligram of lung tissue) measured from a portion of the lung tissue harvested from each mouse remaining on day twenty-one of the bleomycin-induced lung fibrosis study. Data are mean±SEM and analyzed by 1-way ANOVA test as applicable: *p<0.05, **p<0.01, ***p<0.005.

FIG. 9A shows representative images of Masson's trichrome- and hematoxylin and eosin-stained lung sections from mice, obtained after twenty-one days of dosing with bleomycin and Compound 2 or Compound 4, BID. Five mice from each condition were examined.

FIG. 9B shows Ashcroft scores of pathological lung injury, which is illustrated by Masson's trichrome and hematoxylin and eosin (H&E) stainings in FIG. 9A. Data are mean±SEM and analyzed by 1-way ANOVA test as applicable: *p<0.05, **p<0.01, ***p<0.005.

FIG. 10 shows difference in ear thickness between the sham/vehicle (no recombinant mouse (rm-) IL-23) and the other groups treated with rmIL-23. On days zero (before rmIL-23 injection), three, and four (approximately 24 hours after the final hyperplasia induction injection), thickness of ears was measured using calipers.

FIG. 11A shows representative hematoxylin and eosin-stained skin sections of ears from mice, obtained following four daily injections of either rmIL-23 or PBS and dosing with Compound 2 or Compound 4, BID. Ten mice from each condition were examined.

FIG. 11B shows quantification of the epidermal thickness measured from the immunohistochemistry images. Data are mean±SEM and analyzed by T-test as applicable: *p<0.05, **p<0.01, ***p<0.005.

FIG. 12A shows IL17A cytokine concentration in frozen ear tissue from mice euthanized on day four, approximately 24 hours after the final hyperplasia-induction injection, as measured in each ear sample. Data are mean±SEM and analyzed by T-test as applicable: *p<0.05, **p<0.01, ***p<0.005.

FIG. 12B shows IL-22 cytokine concentration in frozen ear tissue from mice euthanized on day four, approximately 24 hours after the final hyperplasia-induction injection, as measured in each ear sample. Data are mean±SEM and analyzed by T-test as applicable: *p<0.05, **p<0.01, ***p<0.005.

### DETAILED DESCRIPTION

A description of example embodiments follows.

### Compounds

A first embodiment is a compound having the following structural formula:

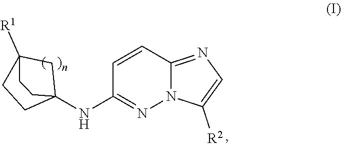

or a pharmaceutically acceptable salt thereof, wherein:
R¹ is —H, —OH, (C₁-C₃)alkyl or (C₁-C₃)hydroxyalkyl;
R² is (C₅-C₆)heteroaryl optionally substituted with one or more R²⁰;
each R²⁰ is independently —OH, halo, —CN, (C₁-C₃)alkyl, (C₁-C₃)haloalkyl, (C₃)carbocyclyl, —NH₂, —N(H)S(O)₂(C₁-C₃)alkyl or —N(CH₃)₂; and
n is 1 or 2.

In a first aspect of the first embodiment, R¹ is —OH or (C₁-C₃)hydroxyalkyl. Values for the remaining variables are as described in the first embodiment.

In a second aspect of the first embodiment, R¹ is —OH or —C(CH₃)₂OH. Values for the remaining variables are as described in the first embodiment, or first aspect thereof.

In a third aspect of the first embodiment, R² is (C₅)heteroaryl optionally substituted with one or more R²⁰. Values for the remaining variables are as described in the first embodiment, or first or second aspect thereof.

In a fourth aspect of the first embodiment, R² is pyrazolyl (and, in some further aspects, 1H-pyrazol-3-yl) or pyrrolyl (and, in some further aspects, 1H-pyrrol-3-yl) optionally substituted with one or more R²⁰. Values for the remaining variables are as described in the first embodiment, of first through third aspects thereof.

In a fifth aspect of the first embodiment, R² is 1H-pyrazol-3-yl or 1H-pyrrol-3-yl optionally substituted with one or more R²⁰. Values for the remaining variables are as described in the first embodiment, of first through fourth aspects thereof.

In a sixth aspect of the first embodiment, each R²⁰ is independently halo, —CN, (C₁-C₃)alkyl, (C₁-C₃)haloalkyl or cyclopropyl. Values for the remaining variables are as described in the first embodiment, or first through fifth aspects thereof.

In a seventh aspect of the first embodiment, each R²⁰ is independently (C₁-C₃)alkyl or cyclopropyl. Values for the remaining variables are as described in the first embodiment, or first through sixth aspects thereof.

In an eighth aspect of the first embodiment, each R²⁰ is independently methyl, ethyl or cyclopropyl. Values for the remaining variables are as described in the first embodiment, or first through seventh aspects thereof.

In a ninth aspect of the first embodiment, n is 1. Values for the remaining variables are as described in the first embodiment, or first through eighth aspects thereof.

In a tenth aspect of the first embodiment, n is 2. Values for the remaining variables are as described in the first embodiment, or first through ninth aspects thereof.

In an eleventh aspect of the first embodiment, R² is substituted with one R²⁰. Values for the variables are as described in the first embodiment, or first through tenth aspects thereof.

A second embodiment is a compound of the following structural formula:

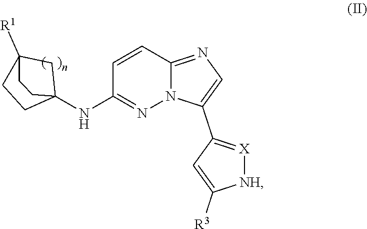

or a pharmaceutically acceptable salt thereof, wherein:
X is N or C(H); and
R³ is —H, —OH, halo, —CN, (C₁-C₃)alkyl, (C₁-C₃)haloalkyl, (C₃)carbocyclyl, —NH₂, —N(H)S(O)₂CH₃ or N(CH₃)₂.
   Values for the remaining variables (e.g., R¹, n) are as described in the first embodiment, or any aspect thereof.

In a first aspect of the second embodiment, X is N. Values for the remaining variables are as described in the first embodiment, or any aspect thereof, or the second embodiment.

In a second aspect of the second embodiment, X is C(H). Values for the remaining variables are as described in the first embodiment, or any aspect thereof, or the second embodiment.

In a third aspect of the second embodiment, R³ is —H, halo, —CN, (C₁-C₃)alkyl, (C₁-C₃)haloalkyl or cyclopropyl. Values for the remaining variables are as described in the first embodiment, or any aspect thereof, or the second embodiment, or first or second aspect thereof.

In a fourth aspect of the second embodiment, R³ is (C₁-C₃)alkyl or cyclopropyl. Values for the remaining variables are as described in the first embodiment, or any aspect thereof, or the second embodiment, or first through third aspects thereof.

In a fifth aspect of the second embodiment, R³ is methyl, ethyl or cyclopropyl. Values for the remaining variables are as described in the first embodiment, or any aspect thereof, or the second embodiment, or first through fourth aspects thereof.

Representative examples of compounds of Structural Formulas (I) and (II) are depicted in Table 1.

In some embodiments, the compound is:

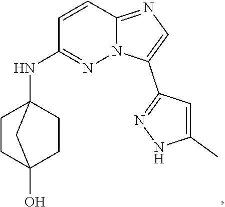

or a pharmaceutically acceptable salt thereof.

In some embodiments, the compound is:

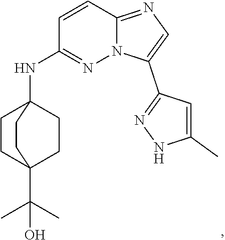 [SMILES: Cc1cc(-c2cnc3ccc(NC45CCC(C(C)(C)O)(CC4)CC5)nn23)n[nH]1]

or a pharmaceutically acceptable salt thereof.

In some embodiments, the compound is:

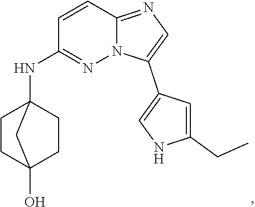 [SMILES: CCc1cc(-c2cnc3ccc(NC45CCC(O)(CC4)C5)nn23)c[nH]1]

or a pharmaceutically acceptable salt thereof.

In some embodiments, the compound is:

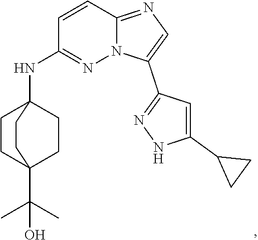 [SMILES: CC(C)(O)C12CCC(Nc3ccc4ncc(-c5cc(C6CC6)[nH]n5)n4n3)(CC1)CC2]

or a pharmaceutically acceptable salt thereof.

In some embodiments, the compound is:

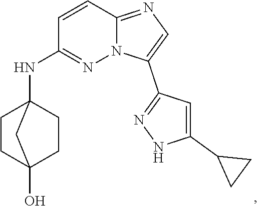 [SMILES: OC12CCC(Nc3ccc4ncc(-c5cc(C6CC6)[nH]n5)n4n3)(CC1)C2]

or a pharmaceutically acceptable salt thereof.

In some embodiments, the compound is:

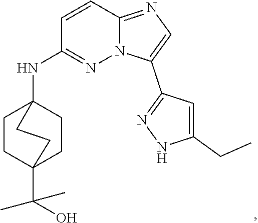 [SMILES: CCc1cc(-c2cnc3ccc(NC45CCC(C(C)(C)O)(CC4)CC5)nn23)n[nH]1]

or a pharmaceutically acceptable salt thereof.

In some embodiments, the compound is:

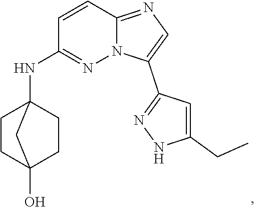 [SMILES: CCc1cc(-c2cnc3ccc(NC45CCC(O)(CC4)C5)nn23)n[nH]1]

or a pharmaceutically acceptable salt thereof.

### Definitions

For purposes of interpreting this specification, the following definitions will apply, and whenever appropriate, terms used in the singular will also include the plural. Terms used in the specification have the following meanings unless the context clearly indicates otherwise.

All methods described herein can be performed in any suitable order unless otherwise indicated herein or otherwise clearly contradicted by context. The use of any and all examples, or exemplary language (e.g., “such as”) provided herein is intended merely to better illuminate the present disclosure and does not pose a limitation on the scope of the present disclosure otherwise claimed.

The terms “a,” “an,” “the” and similar terms used in the context of the present disclosure (especially in the context of the claims) are to be construed to cover both the singular and plural unless otherwise indicated herein or clearly contradicted by the context.

As used herein, the term “heteroatom” refers to nitrogen (N), oxygen (O) or sulfur (S) atoms, in particular, nitrogen or oxygen. When one heteroatom is S, it can be optionally mono- or di-oxygenated (i.e., —S(O)— or —S(O)₂). Unless otherwise indicated, any heteroatom with unsatisfied valencies is assumed to have hydrogen atoms sufficient to satisfy the valencies.

As used herein, the term “alkyl” refers to a branched or straight-chain, monovalent, hydrocarbon radical having the specified number of carbon atoms, and the general formula CₙH₂ₙ₊₁. Thus, the term “(C₁-C₆)alkyl” refers to a branched or straight-chain, monovalent, hydrocarbon radical of the general formula CₙH₂ₙ₊₁ wherein n is 1, 2, 3, 4, 5 or 6. Examples of alkyl include, but are not limited to, methyl, ethyl, n-propyl, i-propyl, n-butyl, i-butyl, s-butyl, t-butyl, n-pentyl, 1-methylbutyl, 2-methylbutyl, 3-methylbutyl, neopentyl, 3,3-dimethylpropyl, hexyl, 2-methylpentyl, and the like.

“Halogen” and “halo,” as used herein, refer to fluorine, chlorine, bromine or iodine. In some embodiments, halogen is fluoro, chloro or bromo. In some embodiments, halogen is fluoro or chloro. In some embodiments, halogen is chloro, bromo or iodo. In some embodiments, halogen is chloro or bromo.

“Haloalkyl,” as used herein, refers to an alkyl radical wherein one or more hydrogen atoms is each independently replaced by a halogen, wherein alkyl and halogen are as described herein. “Haloalkyl” includes mono-, poly- and perhaloalkyl groups. “(C₁-C₆)haloalkyl” refers to a (C₁-C₆)alkyl wherein one or more hydrogen atoms is each independently replaced by a halogen. Examples of haloalkyl include, but are not limited to, fluoromethyl, difluoromethyl, trifluoromethyl, trichloromethyl, pentafluoroethyl, pentachloroethyl, 2,2,2-trifluoroethyl, heptafluoropropyl, and heptachloropropyl.

“Hydroxyalkyl,” as used herein, refers to an alkyl radical wherein one or more (e.g., one) hydrogen atoms is replaced by a hydroxy, wherein alkyl and hydroxy are as described herein. “Hydroxy(C₁-C₆)alkyl” refers to a (C₁-C₆)alkyl wherein one or more hydrogen atoms is replaced by a hydroxy. Examples of hydroxyalkyl include, but are not limited to, 2-hydroxyethyl and 2-hydroxypropyl.

The term “carbocyclyl,” as used herein, refers to a saturated or unsaturated, non-aromatic, monocyclic or polycyclic (e.g., bicyclic, tricyclic) hydrocarbon ring system having the specified number of ring carbon atoms. Thus, “(C₅-C₈)carbocyclyl” means a carbocyclyl ring system having from 5 to 8 ring carbons. A carbocyclyl can be saturated (i.e., a cycloalkyl). Alternatively, a carbocyclyl can be unsaturated (i.e., contain at least one degree of unsaturation, as in at least one carbon-carbon double bond or triple bond). A carbocyclyl ring system may consist of monocyclic rings, fused rings, bridged rings and spirocyclic rings. Examples of carbocyclyl include, but are not limited to, cyclopropyl, cyclobutyl, cyclopentyl, cyclopentenyl, cyclohexyl, cyclohexenyl, bicyclo[2.2.1]heptanyl, bicyclo[2.2.2]octanyl, and norbornyl.

The term “cycloalkyl,” as used herein, refers to a saturated, monocyclic or polycyclic (e.g., bicyclic, tricyclic), aliphatic, hydrocarbon ring system having the specified number of carbon atoms. Thus, “(C₅-C₈)cycloalkyl” means a cycloalkyl ring system having from 5 to 8 ring carbons. Examples of cycloalkyl include, but are not limited to, cyclopropyl, cyclobutyl, cyclopentyl, cyclohexyl, bicyclo[2.2.1]heptanyl, bicyclo[2.2.2]octanyl, and norbornyl.

The term “heteroaryl,” as used herein, refers to a monocyclic or polycyclic, aromatic, hydrocarbon ring system having the specified number of ring atoms, wherein at least one carbon atom in the ring has been replaced with a heteroatom. Thus, “(C₅-C₆)heteroaryl” refers to a heteroaryl ring system having five or six ring atoms. Typically, heteroaryl has 5 to 15, 5 to 10, 5 to 9, or 5 to 6 ring atoms. A heteroaryl ring system may consist of a single ring or a fused ring system. A typical monocyclic heteroaryl is a 5- to 6-membered ring containing one to three heteroatoms (e.g., one, two or three) independently selected from oxygen, sulfur and nitrogen, and a typical fused heteroaryl ring system is a 9- to 10-membered ring system containing one to four heteroatoms independently selected from oxygen, sulfur and nitrogen. The fused heteroaryl ring system may consist of two heteroaryl rings fused together or a heteroaryl ring fused to an aryl ring (e.g., phenyl). Examples of heteroaryl include, but are not limited to, pyrrolyl, pyridyl, pyrazolyl, indolyl, indolinyl, isoindolinyl, indazolyl, thienyl, furanyl, benzofuranyl, dihydrobenzofuranyl, dihydroisobenzofuranyl, oxazolyl, isoxazolyl, imidazolyl, triazolyl, tetrazolyl, triazinyl, pyrimidinyl, pyrazinyl, thiazolyl, purinyl, benzimidazolyl, quinolinyl, isoquinolinyl, quinoxalinyl, tetrahydroquinolinyl, benzofuranyl, benzopyranyl, benzothiophenyl, benzoimidazolyl, benzoxazolyl, 1H-benzo[d][1,2,3]triazolyl, and the like.

The term “substituted,” as used herein, means that at least one (e.g., one, two, three, four, five, six, etc., from one to five, from one to three, one or two) hydrogen atom is replaced with a non-hydrogen substituent, provided that normal valencies are maintained and that the substitution results in a stable compound. Unless otherwise indicated, an “optionally substituted” group can have a substituent at each substitutable position of the group. When more than one position in any given structure may be substituted with more than one substituent selected from a specified group, the substituent can be the same or different at every position. Alternatively, an “optionally substituted group” can be unsubstituted.

When a substituent is oxo, then two hydrogens on a single atom are replaced with the substituent. Oxo substituents are not present on aromatic moieties.

When there is a nitrogen atom(s) on a compound of the present disclosure, the nitrogen atom(s) may be independently converted to N-oxides by treatment with an oxidizing agent (e.g., mCPBA and/or hydrogen peroxide) to afford other compounds of the present disclosure. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 substituents, then said group may be unsubstituted or substituted with up to three substituents, and each substituent is selected independently from the other substituent(s).

When a bond to a substituent is shown to cross a bond connecting two atoms in a ring (as the bond to R¹ in structural formula I) or to cross a circle denoting a ring (as the bond to R³ in structural formula I), then such substituent may be bonded to any substitutable atom in the ring. Further, when the ring the bond to the substituent crosses into is polycyclic (e.g., bicyclic, as in the ring system in structural formula I containing X and Y), the substituent may be bonded to any substitutable atom of the ring or ring system the bond to the substituent crosses into. When a substituent is listed without indicating the atom to which such substituent is bonded to the rest of the compound of a given formula, then such substituent may be bonded via any atom in such substituent.

Combinations of substituents and/or variables are permissible only if such combinations result in stable compounds.

As a person of ordinary skill in the art would understand, for example, a ketone (—C(H)C(O)) group in a molecule may tautomerize to its enol form (—C═C(OH)). This disclosure is intended to cover all possible tautomers even when a structure depicts only one of them.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

Unless specified otherwise, the term “compounds of the present disclosure” refers to a compound of any structural formula depicted herein (e.g., a compound of Formula I, a subformula of a compound of Formula I), as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts.

Compounds of the present disclosure may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemic mixtures, individual isomers (e.g., diastereomers, enantiomers, geometrical isomers, conformational isomers (including rotamers and atropisomers), tautomers) and intermediate mixtures, with all possible isomers and mixtures thereof being included in the present invention.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and (S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

The compounds of the present disclosure can be isolated in optically active or racemic forms. Optically active forms may be prepared by resolution of racemic forms or by synthesis from optically active starting materials. All processes used to prepare compounds of the present disclosure and intermediates made therein are considered to be part of the present disclosure. When enantiomeric or diastereomeric products are prepared, they may be separated by conventional methods, for example, by chromatography or fractional crystallization.

Depending on the process conditions, the end products of the present disclosure are obtained either in free (neutral) or salt form. Both the free form and the salts of these end products are within the scope of the present disclosure. If so desired, one form of a compound may be converted into another form. A free base or acid may be converted into a salt; a salt may be converted into the free compound or another salt; a mixture of isomeric compounds of the present disclosure may be separated into the individual isomers.

Pharmaceutically acceptable salts are preferred. However, other salts may be useful, e.g., in isolation or purification steps which may be employed during preparation, and thus, are contemplated to be within the scope of the present disclosure.

As used herein, “pharmaceutically acceptable salts” refers to salts derived from suitable inorganic and organic acids and inorganic and organic bases that are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable acid addition salts can be formed from inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable acid addition salts include, but are not limited to, acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethanedisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate/hydroxymalonate, mandelate, mesylate, methylsulphate, mucate, naphthoate, napsylate, nicotinate, nitrate, octadecanoate, oleate, oxalate, palmitate, pamoate, phenylacetate, phosphate/hydrogen phosphate/dihydrogen phosphate, polygalacturonate, propionate, salicylates, stearate, succinate, sulfamate, sulfosalicylate, tartrate, tosylate, trifluoroacetate and xinafoate salts.

Pharmaceutically acceptable base addition salts can be formed from inorganic and organic bases. Inorganic bases from which salts can be derived include, for example, ammonium salts and metals from columns I to XII of the periodic table. In certain embodiments, a base addition salt is derived from sodium, potassium, ammonium, calcium, magnesium, iron, silver, zinc, or copper; particularly suitable base addition salts include ammonium, potassium, sodium, calcium and magnesium salts. Organic bases from which salts can be derived include, for example, primary, secondary, and tertiary amines, substituted amines including naturally occurring substituted amines, cyclic amines, basic ion-exchange resins, and the like. Examples of organic amines include, but are not limited to, isopropylamine, benzathine, cholinate, diethanolamine, diethylamine, lysine, meglumine, piperazine and tromethamine.

A salt (e.g., pharmaceutically acceptable salt) of a compound of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

It will be understood that when the compound of the present disclosure contains more than one basic moiety or more than one acidic moiety, each such moiety can independently be involved in forming an acid addition salt form or base addition salt form, with all possible salt forms being included in this disclosure. Further, when two or more moieties of a compound of the present disclosure are in salt form, the anions or cations forming the two or more salt forms can be the same or different. Typically, the anions or cations forming the two or more salt forms are the same. Typical molar ratios of an anion or cation in a salt of a compound of the present disclosure to the compound of the present disclosure are 3:1, 2:1, 1:1, 2:1, 3:1, 4:1 and 5:1. In some embodiments, the molar ratio of an anion or cation (e.g., anion) in a salt of a compound of the present disclosure to the compound of the present disclosure is 1:1 (e.g., as in the monohydrochloride salt of Compound 6). In some embodiments, the molar ratio of an anion or cation (e.g., anion) in a salt of a compound of the present disclosure to the compound of the present disclosure is 2:1 (e.g., as in the dihydrochloride salt of Compound 4).

Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorous, fluorine, chlorine and iodine, such as ²H, ³H, ¹¹C, ¹³C, ¹⁴C, ¹⁵N, ¹⁸F, ³¹P, ³²P, ³⁵S, ³⁶Cl, ¹²³I, ¹²⁴I and ¹²⁵I, respectively. The present disclosure includes various isotopically labeled compounds as defined herein, for example those into which radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies.

Further, substitution with heavier isotopes, particularly deuterium (i.e., ²H or D) may afford certain therapeutic advantages resulting from greater metabolic stability, for example, increased in vivo half-life or reduced dosage requirements or an improvement in therapeutic index. It is understood that deuterium in this context is regarded as a substituent of a compound of the present disclosure. The concentration of such a heavier isotope, specifically deuterium, may be defined by the isotopic enrichment factor. The term “isotopic enrichment factor,” as used herein, means the ratio between the isotopic abundance and the natural abundance of a specified isotope. If a substituent in a compound of this present disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Isotopically labeled compounds of the present disclosure can generally be prepared by conventional techniques known to those skilled in the art or by processes disclosed in the schemes or in the examples and preparations described below (or analogous processes to those described hereinbelow), by substituting an appropriate or readily available isotopically labeled reagent for a non-isotopically labeled reagent otherwise employed. Such compounds have a variety of potential uses, e.g., as standards and reagents in determining the ability of a potential pharmaceutical compound to bind to target proteins or receptors, or for imaging compounds of this disclosure bound to biological receptors in vivo or in vitro.

The term “solvate” means a physical association of a compound of the present disclosure with one or more solvent molecules, whether organic or inorganic. This physical association includes hydrogen bonding. In certain instances, the solvate will be capable of isolation, for example, when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution phase and solid phase solvates. Examples of solvates include, but are not limited to, hydrates, ethanolates, methanolates, and isopropanolates. Methods of solvation are generally known in the art.

Compounds of the present disclosure can be provided as amorphous solids or crystalline solids. Lyophilization can be employed to provide the compounds of the present disclosure as a solid.

The terms “malignancy” and “cancer” are used interchangeably herein, and refer to diseases in which abnormal cells divide without control and can invade nearby tissues. Malignant cells can also spread to other parts of the body through the blood and lymph systems. There are several main types of malignancy. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that starts in blood-forming tissue, such as the bone marrow, and causes large numbers of abnormal blood cells to be produced and enter the blood. Lymphoma and multiple myeloma are malignancies that begin in the cells of the immune system. Central nervous system cancers are malignancies that begin in the tissues of the brain and spinal cord.

The term “solid tumor,” as used herein, refers to malignancies/cancers formed of abnormal masses of tissue that usually do not contain cysts or liquid areas. Solid tumors are named/classified according to the tissue/cells of origin. Examples include, but are not limited to, sarcomas and carcinomas.

The term “leukemia,” as used herein, refers to hematologic or blood cell malignancies/cancers that begin in blood-forming tissue, such as the bone marrow. Examples include, but are not limited to, chronic leukemia, acute leukemia, acute myeloid leukemia (AML), chronic myeloid leukemia (CML), acute lymphocytic leukemia (ALL), acute lymphoblastic leukemia (e.g., B-cell, T-cell) and chronic lymphocytic leukemia (CLL).

The term “lymphoma,” as used herein, refers to lymphatic cell malignancies/cancers that begin in the cells of the immune system. Examples include, but are not limited to, Hodgkin's lymphoma, non-Hodgkin's lymphoma and multiple myeloma.

As used herein, the term “subject” refers to an animal. Typically, the animal is a mammal. A subject also refers to, for example, primates (e.g., humans), cows, sheep, goats, horses, dogs, cats, rabbits, rats, mice, fish, birds and the like. In certain embodiments, the subject is a primate. In yet other embodiments, the subject is a human. In yet other embodiments, the subject (e.g., human) does not have cancer (e.g., a fibrotic cancer).

As used herein, a subject (e.g., a human) is “in need of” a treatment if such subject would benefit biologically, medically or in quality of life from such treatment.

“Treat,” “treating” and “treatment,” as used herein, refer to the administration of a medication or medical care to a subject, such as a human, having a disease or condition of interest, e.g., a cancer, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; and/or (iv) relieving the symptoms resulting from the disease or condition (e.g., pain, weight loss, cough, fatigue, weakness, etc.).

As used herein, “first-line therapy” refers to the first therapy given for a disease or condition.

As used herein, “subsequent therapy” refers to any therapy given after a first-line therapy for a disease or condition. When a first-line therapy includes therapeutic agent(s), a subsequent therapy comprises one or more therapeutic agent(s) that are different from the therapeutic agent(s) of a first-line therapy. In some embodiments, the subsequent therapy is a second-line therapy (i.e., the second therapy given for a disease or condition). In some embodiments, the subsequent therapy is a third-line therapy (i.e., the third therapy given for a disease or condition). In some embodiments, the subsequent therapy is a last-line therapy.

In some embodiments, the cancer is resistant. As used herein, “resistant cancer”, “cancer is resistant” and “refractory cancer” refers to cancer that does not respond to a treatment. For example, the cancer may be resistant at the beginning of treatment, or it may become resistant during treatment. The treatment resistance can be occurred by different mechanisms and examples include individual genetic differences, multi-drug resistance, cell death inhibiting (apoptosis suppression), altering in the drug metabolism, epigenetic and drug targets, enhancing DNA repair and gene amplification.

The term “a therapeutically effective amount,” as used herein, refers to an amount of a therapeutic agent, such as a compound of the present disclosure, that, when administered to a subject, such as a human, is sufficient to effect treatment. The amount of a therapeutic agent that constitutes an “effective amount” will vary depending on the therapeutic agent, the condition being treated and its severity, the manner of administration, the duration of treatment, or the subject to be treated (e.g., age, weight, fitness of the subject), but can be determined routinely by one of ordinary skill in the art based on his own knowledge and this disclosure. In embodiments, an “effective amount” effects treatment as measured by a statistically significant change in one or more indications, symptoms, signs, diagnostic tests, vital signs, and the like. In other embodiments, an “effective amount” manages or prevents a condition as measured by a lack of a statistically significant change in one or more indications, symptoms, signs, diagnostic tests, vital signs, and the like.

The regimen of administration can affect what constitutes a therapeutically effective amount. A compound of the present disclosure can be administered to the subject either prior to or after the onset of a cancer condition. Further, several divided dosages, as well as staggered dosages, can be administered daily or sequentially, or the dose can be continuously infused, or can be a bolus injection. Further, the dosages of the compound(s) of the present disclosure can be proportionally increased or decreased as indicated by the exigencies of the therapeutic or prophylactic situation.

### Pharmaceutical Compositions and Combinations

Compounds of the present disclosure are typically used in a pharmaceutical composition (e.g., a pharmaceutical composition comprising a compound of the present disclosure, or a pharmaceutically acceptable salt thereof, and one or more pharmaceutically acceptable carriers). A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

In one aspect, provided herein is a pharmaceutical composition comprising a compound of the present disclosure (e.g., a compound of Formula I, or a subformula thereof, or a pharmaceutically acceptable salt of the foregoing), and a pharmaceutically acceptable carrier (e.g., at least two pharmaceutically acceptable carriers, such as those described herein). In a further aspect, provided herein is a pharmaceutical composition comprising a therapeutically effective amount of a compound of the present disclosure (e.g., a compound of Formula I, or a subformula thereof, or a pharmaceutically acceptable salt of the foregoing), and a pharmaceutically acceptable carrier (e.g., at least two pharmaceutically acceptable carriers, such as those described herein). For purposes of the present disclosure, unless designated otherwise, solvates are generally considered compositions. Preferably, pharmaceutically acceptable carriers are sterile.

The pharmaceutical composition can be formulated for a particular route(s) of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred route of administration can vary depending on the particular compound chosen. In some embodiments, the pharmaceutical composition is formulated for oral administration. In some embodiments, the pharmaceutical composition is formulated for intravenous administration.

Pharmaceutical compositions of the present disclosure can be made up in a solid form (including, without limitation, capsules, tablets, pills, granules, powders or suppositories), or in a liquid form (including, without limitation, solutions, suspensions or emulsions). The pharmaceutical compositions can be subjected to conventional pharmaceutical operations, such as sterilization, and/or can contain conventional inert diluents, lubricating agents, or buffering agents, as well as adjuvants, such as preservatives, stabilizers, wetting agents, emulsifiers and buffers, etc. Typically, the pharmaceutical compositions are tablets or gelatin capsules comprising the active ingredient together with one or more of.
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol;
c) binders, e.g., magnesium aluminum silicate, starch paste, gelatin, tragacanth, methylcellulose, sodium carboxymethylcellulose and/or polyvinylpyrrolidone;
d) disintegrants, e.g., starches, agar, alginic acid or its sodium salt, or effervescent mixtures; and
e) absorbents, colorants, flavors and sweeteners.
   Tablets may be either film-coated or enteric-coated according to methods known in the art.

Suitable compositions for oral administration include a compound of the present disclosure (e.g., a compound of Formula I, or a subformula thereof, or a pharmaceutically acceptable salt of the foregoing) in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions comprise a compound of the present disclosure (e.g., a compound of Formula I, or a subformula thereof, or a pharmaceutically acceptable salt of the foregoing) in the form of an aqueous isotonic solution or suspension, and certain suppositories comprising a compound of the present disclosure (e.g., a compound of Formula I, or a subformula thereof, or a pharmaceutically acceptable salt of the foregoing) are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include a compound of the present disclosure (e.g., a compound of Formula I, or a subformula thereof, or a pharmaceutically acceptable salt of the foregoing) with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Suitable compositions comprising a compound of the present disclosure (e.g., a compound of Formula I, or a subformula thereof, or a pharmaceutically acceptable salt of the foregoing) for topical application, e.g., to the skin and eyes, include aqueous solutions, suspensions, ointments, creams, gels or sprayable formulations, e.g., for delivery by aerosol or the like. Such topical delivery systems will, in particular, be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein, a topical application may also pertain to an inhalation or to an intranasal application. A composition suitable for inhalation or intranasal administration may be conveniently delivered in the form of a dry powder (either alone, as a mixture, for example a dry blend with lactose, or a mixed component particle, for example, with phospholipids) from a dry powder inhaler, or an aerosol spray presentation from a pressurized container, pump, spray, atomizer or nebulizer, with or without the use of a suitable propellant.

The present disclosure further provides anhydrous pharmaceutical compositions and dosage forms comprising a compound provided herein (e.g., a compound of Formula I, or a subformula thereof), or a pharmaceutically acceptable salt thereof, since water may facilitate the degradation of certain compounds. Anhydrous pharmaceutical compositions and dosage forms of the disclosure can be prepared using anhydrous or low moisture-containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The present disclosure further provides pharmaceutical compositions and dosage forms that comprise one or more agents that reduce the rate by which the compound of the present disclosure (e.g., a compound of Formula I, or a subformula thereof, or a pharmaceutically acceptable salt of the foregoing) as an active ingredient will decompose. Such agents, which are referred to herein as “stabilizers,” include, but are not limited to, antioxidants such as ascorbic acid, pH buffers, or salt buffers, etc.

A compound of the present disclosure (e.g., a compound of Formula I, or a subformula thereof, or a pharmaceutically acceptable salt of the foregoing) is typically formulated into pharmaceutical dosage forms to provide an easily controllable dosage of the drug and to give the patient an elegant and easily handleable product. The dosage regimen for the compounds of the present disclosure will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration; the renal and hepatic function of the patient; and the effect desired. Compounds described herein (e.g., a compound of Formula I, or a subformula thereof), or a pharmaceutically acceptable salt thereof, may be administered in a single daily dose, or the total daily dosage may be administered in divided doses, e.g., two, three, or four times daily.

In certain instances, it may be advantageous to administer a compound of the present disclosure (e.g., a compound of Formula I, or a subformula thereof, or a pharmaceutically acceptable salt of the foregoing) in combination with one or more additional therapeutic agent(s). For example, it may be advantageous to administer a compound of the present disclosure (e.g., a compound of Formula I, or a subformula thereof, or a pharmaceutically acceptable salt of the foregoing) in combination with one or more additional therapeutic agents, e.g., independently selected from an anti-cancer agent (e.g., chemotherapeutic agent), anti-allergic agent, anti-emetic, pain reliever, immunomodulator and cytoprotective agent, to treat cancer.

The term “combination therapy” refers to the administration of two or more therapeutic agents to treat a disease, disorder or condition described herein. Such administration encompasses co-administration of the therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Such administration also encompasses use of each type of therapeutic agent in a sequential manner, either at approximately the same time or at different times. The therapeutic agents in a combination therapy can be administered via the same administration route or via different administration routes. Powders and/or liquids may be reconstituted or diluted to a desired dose prior to administration. Typically, the treatment regimen will provide beneficial effects of the drug combination in treating the diseases, conditions or disorders described herein.

Compositions for use in combination therapies will either be formulated together as a pharmaceutical combination, or provided for separate administration (e.g., associated in a kit). Accordingly, provided herein is a pharmaceutical combination comprising a compound of the present disclosure (e.g., a compound of Formula I, or a subformula thereof, or a pharmaceutically acceptable salt of the foregoing) (e.g., a therapeutically effective amount of a compound of the present disclosure), and one or more other therapeutic agents (e.g., a therapeutically effective amount of one or more other therapeutic agents). A pharmaceutical combination can further comprise one or more pharmaceutically acceptable carriers, such as one or more of the pharmaceutically acceptable carriers described herein.

A therapy for use in combination with a compound of the present disclosure can comprise an agent known to modulate other pathway(s) than is(are) modulated by the compound of the present disclosure, other component(s) (e.g., enzymes) of the same pathway(s) as is(are) modulated by the compound of the present disclosure or even one or more of the same targets (e.g., target enzyme(s)) as is(are) modulated by the compound of the present disclosure. In one aspect, a combination therapy comprises a compound of the present disclosure and a chemotherapeutic agent, immunomodulator and/or radiation therapy, for example, to provide a synergistic or additive therapeutic effect.

Examples of therapies for use in combination with a compound of the present disclosure (e.g., in combination therapy, in a pharmaceutical combination) include standard of care therapies and/or regimens (e.g., standard of care agents), such as first-line standard of care therapies (e.g., chemotherapies), intermediate-line standard of care therapies (e.g., chemotherapies) or last-line standard of care therapies (e.g., chemotherapies). Standard of care therapies are therapies that a clinician should use for a certain type of patient, illness and/or clinical circumstance. Often, organizations such as National Comprehensive Cancer Network (NCCN) publish guidelines and/or treatment algorithms setting forth best practices for treatment of certain patients, illnesses and/or clinical circumstances. See nccn.org. These guidelines often establish, set forth and/or summarize standard of care therapies.

In some embodiments, a compound of the present disclosure is administered in combination with a standard of care therapy for fibrosis and/or symptoms of fibrosis. Non-limiting examples of standard of care therapies for fibrosis include nintedanib, pirfenidone and oxygen therapy. In some embodiments, a compound of the present disclosure is administered in combination with nintedanib or pirfenidone, or a pharmaceutically acceptable salt thereof. In some embodiments, a compound of the present disclosure is administered in combination with oxygen therapy.

In some embodiments, a compound of the present disclosure is administered in combination with a standard of care therapy for ovarian cancer. Non-limiting examples of standard of care therapies for ovarian cancer include monotherapy comprising a platinum analogue (e.g., cisplatin, paclitaxel, carboplatin) and combination therapy comprising a platinum analogue (e.g., docetaxel and carboplatin; paclitaxel and carboplatin; carboplatin and liposomal doxorubicin (dox); paclitaxel, carboplatin and bevacizumab (bev); carboplatin and gemcitabine (gem)/(bev); carboplatin, liposomal dox and bev; carboplatin, paclitaxel and bev; cisplatin and gemcitabine; oxaliplatin); targeted therapy, such as antibody therapy (e.g., bevacizumab); therapy comprising a PARP inhibitor (e.g., olaparib, rucaparib, niraparib, veliparib, talazoparib); therapy comprising a tyrosine kinase inhibitor (TKI) (e.g., pazopanib); immunotherapy; therapy comprising an immune checkpoint inhibitor (e.g., PD-1 or PD-L1 inhibitor); a hormone therapy (e.g., tamoxifen, anastrozole, exemestane, letrozole, an LHRH agonist, such as leuprolide acetate, megestrol acetate); and therapy comprising one or more of altretamine, capecitabine, cyclophosphamide, etoposide, ifosfamide, irinotecan, melphalan, paclitaxel (e.g., albumin-bound paclitaxel), pembrolizumab, pemetrexed, sorafenib and vinorelbine.

In some embodiments, a compound of the present disclosure is administered in combination with a standard of care therapy for pancreatic cancer (e.g., advanced pancreatic cancer, pancreatic ductal adenocarcinoma). Non-limiting examples of standard of care therapies for pancreatic cancer include FOLFIRINOX (a chemotherapy regimen made up of folinic acid, bolus fluorouracil, irinotecan and oxaliplatin); modified FOLFIRINOX regimen (a chemotherapy regimen made up of folinic acid, continuous infusion fluorouracil, irinotecan and oxaliplatin); gemcitabine and abraxane; gemcitabine and capecitabine; olaparib; emcitabine and erlotinib; gemcitabine, docetaxel and capecitabine; larotrectinib; pembrolizumab; gemcitabine; and triple combination therapy of nab-paclitaxel, gemcitabine and cisplatin.

In some embodiments, a compound of the present disclosure is administered in combination with a standard of care therapy for prostate cancer, such as castration resistant prostate cancer. Non-limiting examples of standard of care therapies for prostate cancer include therapy comprising a PARP inhibitor (e.g., olaparib, rucaparib, niraparib, veliparib, talazoparib); LHRH agonist (e.g., goserelin acetate, histrelin acetate, leuprolide acetate, and triptorelin pamoate); LHRH antagonist (e.g., degarelix); anti-androgen (e.g., bicalutamide, flutamide, nilutamide, enzalutamide, apalutamide, darolutamide); corticosteroid (e.g., prednisone, methylprednisolone, hydrocortisone, dexamethasone); estrogen (e.g., diethylstilbestrol); and/or androgen synthesis inhibitor (e.g., ketoconazole, abiraterone acetate); and androgen deprivation therapy.

In some embodiments, a compound of the present disclosure is administered in combination with a standard of care therapy for multiple myeloma. Non-limiting examples of standard of care therapies for multiple myeloma include therapy comprising a proteasome inhibitor (e.g., bortezomib, carfilzomib, marizomib).

In some embodiments, a compound of the present disclosure is administered in combination with radiation therapy. Non-limiting examples of radiation therapy include external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g., At211, I131, I125, Y90, Re186, Re188, Sm153, Bi212, P32, and radioactive isotopes of Lu). Suitable radiation sources for use as a cell conditioner include both solids and liquids. By way of non-limiting example, the radiation source can be a radionuclide, such as I125, I131, Yb169, Ir192 as a solid source, I125 as a solid source, or other radionuclides that emit photons, beta particles, gamma radiation, or other therapeutic rays. The radioactive material can also be a fluid made from any solution of radionuclide(s), e.g., a solution of I125 or I131, or a radioactive fluid can be produced using a slurry of a suitable fluid containing small particles of solid radionuclides, such as Au198, Y90. Moreover, the radionuclide(s) can be embodied in a gel or radioactive microspheres.

Compounds of the present disclosure may be effective in sensitizing abnormal cells to radiation therapy. Thus, also provided herein is a method for sensitizing abnormal cells in a subject (e.g., subject in need thereof) to treatment with radiation, comprising administering to the subject an amount of a compound of the present disclosure effective to sensitize abnormal cells to treatment with radiation. The amount of a compound of the present disclosure effective to sensitize abnormal cells to treatment with radiation can be determined by a person of ordinary skill in the art, for example, according to the means for ascertaining effective amounts described herein.

In some embodiments, standard of care therapy includes radiation therapy.

DNA damaging agents can also be used in combination with a compound of the present disclosure. As used herein, “DNA damaging agent” refers to any agent that directly or indirectly damages DNA in such a way that homologous recombination could repair the damage. Non-limiting examples of DNA damaging agents are DNA damaging chemicals, chemotherapeutic agents, radiochemotherapy and ionizing or ultraviolet radiation. Non-limiting examples of DNA damaging chemotherapeutic agents include alkylating agents, nitrosoureas, anti-metabolites, plant alkaloids, plant extracts and radioisotopes. Non-limiting examples of DNA damaging chemotherapeutic agents also include DNA-damaging drugs, for example, 5-fluorouracil (5-FU), capecitabine, gemcitabine, temozolomide, S-1 (Tegafur, 5-chloro-2,4-dihydroxypyridine and oxonic acid), 5-ethynyluracil, arabinosylcytosine (ara-C), 5-azacytidine (5-AC), 2′,2′-difluoro-2′-deoxycytidine (dFdC), purine antimetabolites (e.g., mercaptopurine, azathiopurine, thioguanine), gemcitabine hydrochlorine (Gemzar), pentostatin, allopurinol, 2-fluoro-arabinosyl-adenine (2F-ara-A), hydroxyurea, sulfur mustard (bischloroetyhylsulfide), mechlorethamine, melphalan, chlorambucil, cyclophosphamide, ifosfamide, thiotepa, AZQ, mitomycin C, dianhydrogalactitol, dibromoducitol, alkyl sulfonate (busulfan), nitrosoureas (BCNU, CCNU, 4-methyl CCNU or ACNU), procarbazine, decarbazine, rebeccamycin, anthracyclins such as doxorubicin (adriamycin; ADR), daunorubicin (Cerubicine), idarubicin (Idamycin) and epirubicin (Ellence), anthracyclin analogs such as mitoxantrone, actinomycin D, topoisomerase inhibitors (e.g., non-intercalating topoisomerase inhibitors such as epipodophyllotoxins (etoposide or VP16, teniposide or VM-26)), PARP inhibitors, podophylotoxin, bleomycin (Blea), pepleomycin, compounds that form adducts with nucleic acid including platinum derivatives, e.g., cisplatin (CDDP), trans analog of cisplatin, carboplatin, iproplatin, tetraplatin and oxaliplatin, as well as camptothecin, topotecan, irinotecan (CPT-11), and SN-38. Radiation, e.g., ultraviolet (UV), infrared (IR), or α-, β-, or γ-radiation, is also a DNA damaging agent.

In some embodiments, standard of care therapy includes a DNA damaging agent, such as a DNA crosslinking agent.

Agents that induce endoplasmic reticulum (ER) stress can also be used in combination with a compound of the present disclosure. Non-limiting examples of agents that induce ER stress include agents that increase levels of reactive oxygen species (ROS) (e.g., napabucasin), chaperone inhibitors, HSP90 inhibitors, HSP70 inhibitors, PDI inhibitors and proteasome inhibitors. Further non-limiting examples of agents that induce ER stress include GSK2606414, GSK2656157, STF-083010, TKI (e.g., sorafenib), phosphor-eif2α phosphatase (e.g., Sal003), diindolylmethane derivatives, proteasome inhibitors (e.g., bortezomib), levistolide A, andrographolide, tolfenamic acid, cantharidin, carnosic acid, casticin, cryptotanshinone, curcumin, flavokawain B, fucoidan, 2-3,4-dihydroxyphenylethanol, 7-dimethoxyflavone, SMIP004 (N-(4-butyl-2-methyl-phenylacetamide), licochalcone A, neferine, paeonol, pardaxin, parthenolide, piperine, polyphenon E, polyphyllin D, resveratrol, dehydrocostuslactone, γ-tocotrienol, Ω-hydroxyundec-9-enoic acid, ampelopsin, ardisianone, genistein, guttiferone H, guggulsterone, marchantin M, sarsasapogenin, saxifragifolin, prodigiosin, quercetin, honokiol, brefeldin A, A-tocopheryl succinate, verrucarin A, vitamin E succinate, ultrafine and zerumbone. See, for example, Walczak, A., et al. Oxidative Medicine and Cellular Longevity Volume 2019, Article ID 5729710, the entire content of which is incorporated herein by reference.

Anti-cancer agents of particular interest for use in combination with the compounds of the present disclosure include:

Topoisomerase inhibitors, including Type I topoisomerase inhibitors, such as irinotecan, topotecan, and camptothecin, and Type 2 topoisomerase inhibitors, such as etoposide, doxorubicin, and epirubicin.

Poly(ADP-ribose) polymerase (PARP) inhibitors, such as olaparib, rucaparib, niraparib, talazoparib, veliparib, pamiparib and iniparib.

DNA crosslinking agents, such as cisplatin, carboplatin and oxaliplatin.

Agents that increase levels of reactive oxygen species (ROS), such as napabucasin.

PARP inhibitors, such as olaparib, rucaparib, niraparib, veliparib and talazoparib.

Purine antimetabolites and/or inhibitors of de novo purine synthesis, such as pemetrexed (Alimta®), gemcitabine (Gemzar®), 5-fluorouracil (Adrucil®, Carac® and Efudex®), methotrexate (Trexall®), capecitabine (Xeloda®), floxuridine (FUDR®), decitabine (Dacogen®), azacitidine (Vidaza® and Azadine®), 6-mercaptopurine (Purinethol®), cladribine (Leustatin®, Litak® and Movectro®), fludarabine (Fludara®), pentostatin (Nipent®), nelarabine (Arranon®), clofarabine (Clolar® and Evoltra®), and cytarabine (Cytosar®).

Anti-angiogenesis agents, such as matrix metalloproteinase (MMP) inhibitors (e.g., MMP-2 inhibitors, MMP-9 inhibitors), rapamycin, temsirolimus (CCI-779), everolimus (RAD001), sorafenib, sunitinib and bevacizumab, as well as COX-II inhibitors, such as CELEBREX™ (alecoxib), valdecoxib and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Embodiments of MMP-2 and/or MMP-9 inhibitors include those that have little or no activity inhibiting MMP-1. Other embodiments include MMP inhibitors that selectively inhibit MMP-2 and/or MMP-9 relative to other matrix metalloproteinases (e.g., MMP-1, MMP-3, MMP-4, MMP-5, MMP-6, MMP-7, MMP-8, MMP-10, MMP-11, MMP-12 and/or MMP-13). Specific examples of MMP inhibitors useful in some embodiments include AG-3340, RO 323555 and RS 13-0830.

Autophagy inhibitors, such as chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N6-mercaptopurine riboside and vinblastine, as well as antisense or siRNA that inhibits expression of proteins including but not limited to ATG5 (which are implicated in autophagy).

B-cell lymphoma 2 (BCL-2) inhibitors, such as venetoclax.

B-cell receptor signaling antagonists, such as a Bruton's tyrosine kinase (BTK) inhibitor (e.g., ibrutinib).

Bromodomain inhibitors. A bromodomain inhibitor inhibits at least one bromodomain protein, such as Brd2, Brd3, Brd4 and/or BrdT, for example, Brd4. Non-limiting examples of bromodomain inhibitors include JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct. 10; 56(19):7498-500; GlaxoSmithKline), IBET151 (Bioorg. Med. Chem. Lett. 2012 Apr. 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium), CPI-0610 (Constellation Pharmaceuticals). In some embodiments, the bromodomain inhibitor is TG101209, BI2536, OTX015, C244, IBET762, IBET151, or PFI-1.

Histone deacetylase (HDAC) inhibitors. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. Non-limiting examples of HDAC inhibitors include trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct. 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr. 13; 96(8):4592-7), mocetinostat (Bioorg. Med. Chem. Lett. 2008 Feb. 1; 18(3):106771) and valproic acid (EMBO J. 2001 Dec. 17; 20(24):6969-78). In some embodiments, the HDAC inhibitor is panobinostat, vorinostat, MS275, belinostat, SAHA or LBH589.

Epidermal growth factor receptor tyrosine kinase (EGFR) inhibitors, such as erlotinib, osimertinib, cetuximab, gefitinib, necitumumab, lapatinib, neratinib, panitumumab, vandetanib, and necitumumab. A combination of a compound as described herein and an EGFR inhibitor may be useful, for example, in the treatment of cancers that are related to EGFR dysregulation, such as non-small-cell lung cancer (NSCLC), pancreatic cancer, breast cancer, and colon cancer. EGFR may be dysregulated, for example, due to activating mutations in exons 18, 19, 20, or 21. In particular embodiments, the EGFR inhibitor is erlotinib or osimertinib. In particular embodiments, the combination of a compound of the present disclosure and an EGFR inhibitor is used to treat EGFR-mutated NSCLC. In particular embodiments, the combination of a compound of the present disclosure and an EGFR inhibitor is used to treat an EGFR inhibitor-resistant cancer, for example, and the compound of the present disclosure sensitizes the cancer to the EGFR inhibitor.

EGFR antibodies, such as cetuximab (Erbitux®).

Methylthioadenosine phosphorylase (MTAP) inhibitors, such as (3R,4S)-1-((4-amino-5H-pyrrolo[3,2-d]pyrimidin-7-yl)methyl)-4-((methylthio)methyl)pyrrolidin-3-ol (MT-DADMe-Immucillin-A, CAS 653592-04-2).

Methylthioadenosine ((2R,3R,4S,5S)-2-(6-amino-9H-purin-9-yl)-5-((methylthio)methyl)tetrahydrofuran-3,4-diol, CAS 2457-80-9).

Epidermal growth factor receptor (EGFR) inhibitors, such as erlotinib hydrochloride (Tarceva®) and gefitnib (Iressa®).

Mesenchymal-epithelial transition (MET) inhibitors, such as capmatinib (INC280, CAS 1029712-80-8).

Platelet-derived growth factor (PDGF) receptor inhibitors, such as imatinib (Gleevec®); linifanib (N-[4-(3-amino-1H-indazol-4-yl)phenyl]-N′-(2-fluoro-5-methylphenyl)urea, also known as ABT 869, available from Genentech); sunitinib malate (Sutent®); quizartinib (AC220, CAS 950769-58-1); pazopanib (Votrient®); axitinib (Inlyta@); sorafenib (Nexavar®); vargatef (BIBF 1120, CAS 928326-83-4); telatinib (BAY57-9352, CAS 332012-40-5); vatalanib dihydrochloride (PTK787, CAS 212141-51-0); and motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470).

Phosphoinositide 3-kinase (PI3K) inhibitors, such as 4-[2-(1H-indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 4-(trifluoromethyl)-5-(2,6-dimorpholinopyrimidin-4-yl)pyridin-2-amine (also known as BKM120 or NVP-BKM120, and described in PCT Publication No. WO 2007/084786); alpelisib (BYL719); (5Z)-5-[[4-(4-pyridinyl)-6-quinolinyl]methylene]-2,4-thiazolidinedione (GSK1059615, CAS 958852-01-2); 5-[8-methyl-9-(1-methylethyl)-2-(4-morpholinyl)-9H-purin-6-yl]-2-pyrimidinamine (VS-5584, CAS 1246560-33-7); and everolimus (AFINITOR®).

Cyclin-dependent kinase (CDK) inhibitors, such as ribociclib (LEE011, CAS 1211441-98-3); aloisine A; alvocidib (also known as flavopiridol or HMR-1275, 2-(2-chlorophenyl)-5,7-dihydroxy-8-[(3S,4R)-3-hydroxy-1-methyl-4-piperidinyl]-4-chromenone, and described in U.S. Pat. No. 5,621,002); crizotinib (PF-02341066, CAS 877399-52-5); 2-(2-chlorophenyl)-5,7-dihydroxy-8-[(2R,3S)-2-(hydroxymethyl)-1-methyl-3-pyrrolidinyl]-4H-1-benzopyran-4-one, hydrochloride (P276-00, CAS 920113-03-7); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265, CAS 927880-90-8); indisulam (E7070); roscovitine (CYC202); 6-acetyl-8-cyclopentyl-5-methyl-2-(5-piperazin-1-yl-pyridin-2-ylamino)-8H-pyrido[2,3-d]pyrimidin-7-one, hydrochloride (PD0332991); dinaciclib (SCH727965); N-[5-[[(5-tert-butyloxazol-2-yl)methyl]thio]thiazol-2-yl]piperidine-4-carboxamide (BMS 387032, CAS 345627-80-7); 4-[[9-chloro-7-(2,6-difluorophenyl)-5H-pyrimido[5,4-d][2]benzazepin-2-yl]amino]-benzoic acid (MLN8054, CAS 869363-13-3); 5-[3-(4,6-difluoro-1H-benzimidazol-2-yl)-1H-indazol-5-yl]-N-ethyl-4-methyl-3-pyridinemethanamine (AG-024322, CAS 837364-57-5); 4-(2,6-dichlorobenzoylamino)-1H-pyrazole-3-carboxylic acid N-(piperidin-4-yl)amide (AT7519, CAS 844442-38-2); 4-[2-methyl-1-(1-methylethyl)-1H-imidazol-5-yl]-N-[4-(methylsulfonyl)phenyl]-2-pyrimidinamine (AZD5438, CAS 602306-29-6); palbociclib (PD-0332991); and (2R,3R)-3-[[2-[[3-[[S(R)]—S-cyclopropylsulfonimidoyl]-phenyl]amino]-5-(trifluoromethyl)-4-pyrimidinyl]oxy]-2-butanol (BAY 10000394).

p53-MDM2 inhibitors, such as (S)-1-(4-chloro-phenyl)-7-isopropoxy-6-methoxy-2-(4-{methyl-[4-(4-methyl-3-oxo-piperazin-1-yl)-trans-cyclohexylmethyl]-amino}-phenyl)-1,4-dihydro-2H-isoquinolin-3-one, (S)-5-(5-chloro-1-methyl-2-oxo-1,2-dihydro-pyridin-3-yl)-6-(4-chloro-phenyl)-2-(2,4-dimethoxy-pyrimidin-5-yl)-1-isopropyl-5,6-dihydro-1H-pyrrolo[3,4-d]imidazol-4-one, [(4S,5R)-2-(4-tert-butyl-2-ethoxyphenyl)-4,5-bis(4-chlorophenyl)-4,5-dimethylimidazol-1-yl]-[4-(3-methylsulfonylpropyl)piperazin-1-yl]methanone (RG7112), 4-[[(2R,3S,4R,5S)-3-(3-chloro-2-fluorophenyl)-4-(4-chloro-2-fluorophenyl)-4-cyano-5-(2,2-dimethylpropyl)pyrrolidine-2-carbonyl]amino]-3-methoxybenzoic acid (RG7388), SAR299155, 2-((3R,5R,6S)-5-(3-chlorophenyl)-6-(4-chlorophenyl)-1-((S)-1-(isopropylsulfonyl)-3-methylbutan-2-yl)-3-methyl-2-oxopiperidin-3-yl)acetic acid (AMG232), {(3R,5R,6S)-5-(3-chlorophenyl)-6-(4-chlorophenyl)-1-[(2S,3S)-2-hydroxy-3-pentanyl]-3-methyl-2-oxo-3-piperidinyl}acetic acid (AM-8553), (+)-4-[4,5-bis(4-chlorophenyl)-2-(2-isopropoxy-4-methoxy-phenyl)-4,5-dihydro-imidazole-1-carbonyl]-piperazin-2-one (Nutlin-3), 2-methyl-7-[phenyl(phenylamino)methyl]-8-quinolinol (NSC 66811), 1-N-[2-(1H-indol-3-yl)ethyl]-4-N-pyridin-4-ylbenzene-1,4-diamine (JNJ-26854165), 4-[4,5-bis(3,4-chlorophenyl)-2-(2-isopropoxy-4-methoxy-phenyl)-4,5-dihydro-imidazole-1-carboxyl]-piperazin-2-one (Caylin-1), 4-[4,5-bis(4-trifluoromethyl-phenyl)-2-(2-isopropoxy-4-methoxy-phenyl)-4,5-dihydro-imidazole-1-carboxyl]-piperazin-2-one (Caylin-2), 5-[[3-dimethylamino)propyl]amino]-3,10-dimethylpyrimido[4,5-b]quinoline-2,4(3H,10H)-dione dihydrochloride (HLI373) and trans-4-iodo-4′-boranyl-chalcone (SC204072).

Mitogen-activated protein kinase (MEK) inhibitors, such as XL-518 (also known as GDC-0973, CAS No. 1029872-29-4, available from ACC Corp.); selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886, described in PCT Publication No. WO 2003/077914); 2-[(2-chloro-4-iodophenyl)amino]-N-(cyclopropylmethoxy)-3,4-difluoro-benzamide (also known as CI-1040 or PD184352 and described in PCT Publication No. WO 2000/035436); N-[(2R)-2,3-dihydroxypropoxy]-3,4-difluoro-2-[(2-fluoro-4-iodophenyl)amino]-benzamide (also known as PD0325901 and described in PCT Publication No. WO 2002/006213); 2,3-bis[amino[(2-aminophenyl)thio]methylene]-butanedinitrile (also known as U0126 and described in U.S. Pat. No. 2,779,780); N-[3,4-difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766 and described in PCT Publication No. WO 2007/014011); (3S,4R,5Z,8S,9S,11E)-14-(ethylamino)-8,9,16-trihydroxy-3,4-dimethyl-3,4,9,19-tetrahydro-1H-2-benzoxacyclotetradecine-1,7(8H)-dione] (also known as E6201 and described in PCT Publication No. WO 2003/076424); 2′-amino-3′-methoxyflavone (also known as PD98059 available from Biaffin GmbH & Co., KG, Germany); (R)-3-(2,3-dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); trametinib dimethyl sulfoxide (GSK-1120212, CAS 1204531-25-80); 2-(2-fluoro-4-iodophenylamino)-N-(2-hydroxyethoxy)-1,5-dimethyl-6-oxo-1,6-dihydropyridine-3-carboxamide (AZD 8330); 3,4-difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655); and 5-[(4-bromo-2-fluorophenyl)amino]-4-fluoro-N-(2-hydroxyethoxy)-1-methyl-1H-benzimidazole-6-carboxamide (MEK162).

B-RAF inhibitors, such as regorafenib (BAY73-4506, CAS 755037-03-7); tuvizanib (AV951, CAS 475108-18-0); vemurafenib (ZELBORAF®, PLX-4032, CAS 918504-65-1); encorafenib (also known as LGX818); 1-methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-hydroxyethyl)-3-(pyridin-4-yl)-1H-pyrazol-4-yl]-2,3-dihydroinden-1-one oxime (GDC-0879, CAS 905281-76-7); 5-[2-[4-[2-(dimethylamino)ethoxy]phenyl]-5-(4-pyridinyl)-1H-imidazol-4-yl]-2,3-dihydro-1H-inden-1-one oxime (GSK2118436 or SB590885); (+/−)-methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662); dabrafenib (TAFINLAR®); and N-(3-(5-chloro-1H-pyrrolo[2,3-b]pyridine-3-carbonyl)-2,4-difluorophenyl)propane-1-sulfonamide (also known as PLX4720).

ALK inhibitors, such as crizotinib (XALKORI@).

PIM kinase inhibitors, such as:

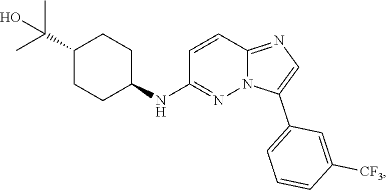 [SMILES: CC(C)(O)[C@H]1CC[C@H](Nc2ccc3ncc(-c4cccc(C(F)(F)F)c4)n3n2)CC1]

or a pharmaceutically acceptable salt thereof.

Proteasome inhibitors, such as bortezomib (VELCADE®), N-5-benzyloxycarbonyl-Ile-Glu(O-tert-butyl)-Ala-leucinal (PSI), carfilzomib and ixazomib, marizomib (NPI-0052), delanzomib (CEP-18770), and O-methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (oprozomib, ONX-0912, PR-047) (e.g., bortezomib), e.g., for the treatment of multiple myeloma.

A host of chemotherapeutic agents can be used in combination with the compound of the present disclosure. In some embodiments, the chemotherapeutic agent is selected from the group consisting of mitotic inhibitors (e.g., paclitaxel, nab-paclitaxel), alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples of chemotherapeutic agents for use in combination with a compound of the present disclosure (e.g., in combination therapy, in a pharmaceutical combination) include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex®, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.), docetaxel (TAXOTERE™, Rhone-Poulenc Rorer, Antony, France) and cabazitaxel (JEVTANA, Sanofi Genzyme); retinoic acid; esperamicins; capecitabine; and pharmaceutically acceptable salts, acids or derivatives of any of the above. Further non-limiting examples of chemotherapeutic agents for use in combination with a compound of the present disclosure (e.g., in combination therapy, in a pharmaceutical combination) include bortezomib, capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), erlotinib, fludarabine phosphate (Fludara®), 5-fluorouracil (Adrucil®, Efudex®), FOLFIRINOX, gemcitabine (difluorodeoxycitidine), irinotecan (Camptosar®), L-asparaginase (ELSPAR®), 6-mercaptopurine (Purinethol®), methotrexate (Folex®), nabpaclitaxel, pentostatin, 6-thioguanine, thiotepa, and topotecan hydrochloride for injection (Hycamptin®). Yet further non-limiting examples of chemotherapeutic agents for use in combination with a compound of the present disclosure (e.g., in combination therapy, in a pharmaceutical combination) include erlotinib, afatinib, gefitinib, GDC0941, MLN1117, BYL719 (alpelisib), BKM120 (buparlisib), CYT387, GLPG0634, baricitinib, lestaurtinib, momelotinib, pacritinib, ruxolitinib, TG101348, crizotinib, tivantinib, AMG337, cabozantinib, foretinib, onartuzumab, NVP-AEW541, dasatinib, ponatinib, saracatinib, bosutinib, trametinib, selumetinib, cobimetinib, PD0325901, RO5126766, axitinib, bevacizumab, cetuximab, fostamatinib, imatinib, lapatinib, lenvatinib, ibrutinib, nilotinib, panitumumab, pazopanib, pegaptanib, ranibizumab, sorafenib, sunitinib, SU6656, trastuzumab, tofacitinib, vandetanib, vemurafenib, irinotecan, Taxol, docetaxel, rapamycin and MLN0128. More non-limiting examples of chemotherapeutic agents for use in combination with a compound of the present disclosure (e.g., in combination therapy, in a pharmaceutical combination) include capecitabine (Xeloda®), N4-pentoxycarbonyl-5-deoxy-5-fluorocytidine, carboplatin (Paraplatin®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), doxorubicin hydrochloride (Adriamycin®, Rubex®), fludarabine phosphate (Fludara®), 5-fluorouracil (Adrucil®, Efudex®), gemcitabine (difluorodeoxycitidine), irinotecan (Camptosar®), L-asparaginase (ELSPAR®), 6-mercaptopurine (Purinethol®), methotrexate (Folex®), pentostatin, 6-thioguanine, thiotepa, and topotecan hydrochloride for injection (Hycamptin®).

Commonly prescribed anti-cancer drugs can also be used in combination with a compound of the present disclosure. Non-limiting examples of commonly prescribed anti-cancer drugs include Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

Chemotherapeutic cell conditioners can also be used in combination with compound of the present disclosure. Non-limiting examples of chemotherapeutic cell conditioners include anti-hormonal agents that act to regulate or inhibit hormone action on tumors, such as anti-estrogens, including, for example, tamoxifen, (Nolvadex™), raloxifene, aromatase inhibiting 4(5)-imidazoles, 4-hydroxytamoxifen, trioxifene, keoxifene, LY 117018, onapristone and toremifene (Fareston); and anti-androgens, such as flutamide, nilutamide, bicalutamide, leuprolide and goserelin; chlorambucil; gemcitabine; 6-thioguanine; mercaptopurine; methotrexate; platinum analogs, such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomeRASe inhibitor RFS 2000; and difluoromethylornithine (DMFO).

mTOR inhibitors can also be used in combination with a compound of the present disclosure. Non-limiting examples of mTOR inhibitors include, e.g., temsirolimus; ridaforolimus (formally known as deferolimus, (1R,2R,4S)-4-[(2R)-2 [(1R,9S,12S,15R,16E,18R,19R,21R,23 S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartyl-L-serine-inner salt (SF1126, CAS 936487-67-1) and XL765.

Some patients may experience allergic reactions to compounds of the present disclosure and/or other therapeutic agent(s) (e.g., anti-cancer agent(s)) during or after administration. Therefore, anti-allergic agents can be administered in combination with compounds of the present disclosure and/or other therapeutic agent(s) (e.g., anti-cancer agent(s)) to minimize the risk of an allergic reaction. Suitable anti-allergic agents include corticosteroids (Knutson, S., et al., PLoS One, DOI:10.1371/journal.pone.0111840 (2014)), such as dexamethasone (e.g., DECADRON®), beclomethasone (e.g., BECLOVENT®), hydrocortisone (also known as cortisone, hydrocortisone sodium succinate, hydrocortisone sodium phosphate, sold under the tradenames ALA-CORT®, hydrocortisone phosphate, SOLU-CORTEF®, HYDROCORT ACETATE® and LANACORT®), prednisolone (sold under the tradenames DELTA-CORTEL®, ORAPRED®, PEDIAPRED® and PRELONE®), prednisone (sold under the tradenames DELTASONE®, LIQUID RED®, METICORTEN® and ORASONE®), methylprednisolone (also known as 6-methylprednisolone, methylprednisolone acetate, methylprednisolone sodium succinate, sold under the tradenames DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

Some patients may experience nausea during and after administration of the compounds described herein and/or other therapeutic agent(s) (e.g., anti-cancer agent(s)). Therefore, anti-emetics can be used in combination with compounds of the present disclosure and/or other therapeutic agent(s) (e.g., anti-cancer agent(s)) to prevent nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and ZUNRISA®), and combinations thereof.

Medication to alleviate the pain experienced during the treatment period is often prescribed to make the patient more comfortable. Common over-the-counter analgesics, such TYLENOL®, can also be used in combination with compounds of the present disclosure and/or other therapeutic agent(s) (e.g., anti-cancer agent(s)). Opioid analgesic drugs such as hydrocodone/paracetamol or hydrocodone/acetaminophen (e.g., VICODIN®), morphine (e.g., ASTRAMORPH® or AVINZA®), oxycodone (e.g., OXYCONTIN® or PERCOCET®), oxymorphone hydrochloride (OPANA®), and fentanyl (e.g., DURAGESIC®) can be useful for moderate or severe pain, and can be used in combination with compounds of the present disclosure and/or other therapeutic agent(s) (e.g., anti-cancer agent(s)).

Immunomodulators can also be used in combination with compounds of the present disclosure. Non-limiting examples of immunomodulators (e.g., immunooncology agents) include afutuzumab (available from ROCHE®); pegfilgrastim (NEULASTA®); lenalidomide (CC-5013, REVLIMID®); thalidomide (THALOMID®); actimid (CC4047); and IRX-2 (mixture of human cytokines including interleukin 1, interleukin 2, and interferon 7, CAS 951209-71-5, available from IRX Therapeutics).

Immunomodulators of particular interest for use in combination with the compounds of the present disclosure include:

Chimeric antigen receptor T-cell (CAR-T) therapies, such as tisagenlecleucel (Novartis), axicabtagene ciloleucel (Kite), and tocilizumab (atlizumab; Roche).

Immune checkpoint inhibitors, such as PD-1 inhibitors, PD-L1 inhibitors, cytotoxic T-lymphocyte-associated modulators (e.g., CTLA-4 inhibitors), LAG-3 inhibitors, TIM-3 inhibitors.

PD-1 inhibitors, such as pembrolizumab (also known as Lambrolizumab, MK-3475, MK03475, SCH-900475, or KEYTRUDA®) and other anti-PD-1 antibodies (as disclosed in Hamid, O. et al. (2013) New England Journal of Medicine 369 (2): 134-44, U.S. Pat. No. 8,354,509, and WO 2009/114335, incorporated by reference in their entirety), nivolumab (also known as MDX-1106, MDX-1106-04, ONO-4538, BMS-936558, or OPDIVO®) and other anti-PD-1 antibodies (as disclosed in U.S. Pat. No. 8,008,449 and WO 2006/121168, incorporated by reference in their entirety), cemiplimab (LIBTAYO®), spartalizumab (PDR001), pidilizumab (CureTech), MEDI0680 (Medimmune), dostarlimab (TSR-042), PF-06801591 (Pfizer), sinitilimab, toripalimab, tislelizumab (BGB-A317), camrelizumab (INCSHR1210, SHR-1210), AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZM009 (Livzon), HLX-10 (Henlius Biotech), MEDIO680 (Medimmune), PDF001 (Novartis), PF-06801591 (Pfizer), pidilizumab (CureTech) also known as CT-011 and other anti-PD-1 antibodies (as disclosed in Rosenblatt, J. et al. (2011) J Immunotherapy 34(5): 409-18, U.S. Pat. Nos. 7,695,715, 7,332,582, and 8,686,119, incorporated by reference in their entirety), REGN2810 (Regeneron) and TSR-042 (Tesaro), also known as ANBO11, or CS1003 (CStone Pharmaceuticals). MEDIO680 (Medimmune), is also known as AMP-514. MEDIO680 and other anti-PD-1 antibodies are disclosed in U.S. Pat. No. 9,205,148 and WO 2012/145493, incorporated by reference in their entireties. Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entireties. In one embodiment, the PD-1 inhibitor is an anti-PD-1 antibody molecule, as described in US 2015/0210769, published on Jul. 30, 2015, entitled “Antibody Molecules to PD-1 and Uses Thereof,” incorporated by reference in its entirety. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety. In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In one embodiment, the PD-1 inhibitor is AMP-224 (B7-DCIg (Amplimmune), e.g., disclosed in WO 2010/027827 and WO 2011/066342, incorporated by reference in their entireties).

PD-L1 inhibitors, such as atezolizumab (also known as MPDL3280A, RG7446, RO5541267, YW243.55.S70, or TECENTRIQ®) and other anti-PD-L1 antibodies as disclosed in U.S. Pat. No. 8,217,149, incorporated by reference in its entirety, avelumab (BAVENCIO® also known as MSB0010718C) and other anti-PD-L1 antibodies as disclosed in WO 2013/079174, incorporated by reference in its entirety, durvalumab (IMFINZI® or MEDI4736) and other anti-PD-L1 antibodies as disclosed in U.S. Pat. No. 8,779,108, incorporated by reference in its entirety), FAZ053 (Novartis), and BMS-936559 (Bristol-Myers Squibb). In certain embodiments, the PD-L1 inhibitor is KN035 (Alphamab; 3DMed; Ascletis Pharma), Envafolimab (TRACON Pharmaceuticals), BMS 936559 (Bristol-Myers Squibb), CS1001 (CStone Pharmaceuticals, Ligand Pharmaceuticals), CX-072 (CytomX Therapeutics), FAZ053 (Novartis), SHR-1316 (Hengrui Medicine), TQB2450 (Chiatai Tianqing), STI-A1014 (Zhaoke Pharm; Lee's Pharm, Lonza, Sorrento Therapeutics, NantWorks), LYNO0102 (Lynkcell), A167 (Harbour BioMed, Kelun Group), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entireties. In certain embodiments, the PD-L1 inhibitor is cosibelimab (Fortress Biotech), LY3300054 or iodapolimab (Eli Lilly), GS-4224 (Gilead Sciences), STI-A1015 (Yuhan, Sorrento Therapeutics), BCD-135 (BIOCAD), cosibelimab (Dana-Farber Cancer Institute, TG Therapeutics), APL-502 (Apollomics), AK106 (Akeso Biopharma), MSB2311 (Transcenta Holding), TG-1501 (TG Therapeutics) or FAZ053 (Novartis). In certain embodiments, the PD-L1 inhibitor is MT-6035 (Molecular Templates), icaritin or ZKAB001 (Lonza, Lee's Pharmaceutical Holdings, Sorrento Therapeutics, Shenogen Pharma Group), TRIDENT Antibody (MacroGenics, Zai Lab), YBL-007 (Anh-Gook Pharmaceutical, Y-Biologics), HTI-1316 (Hengrui Therapeutics), PD-L1 Oncology Project (Weizmann Institute of Sciences), JS003 (Shanghai Junshi Biosciences), ND021 (Numab Therapeutics, CStone Pharmaceuticals), Toca 521 (Tocagen) or STTO1 (STCube). In certain embodiments, the PD-L1 inhibitor is DB004 (DotBio), MT-5050 (Molecular Templates), KD036 (Kadmon). In one embodiment, the PD-L1 inhibitor is an anti-PD-L1 antibody molecule. In one embodiment, the PD-L1 inhibitor is an anti-PD-L1 antibody molecule as disclosed in US 2016/0108123, published on Apr. 21, 2016, entitled “Antibody Molecules to PD-L1 and Uses Thereof,” incorporated by reference in its entirety. In one embodiment, the anti-PD-L1 antibody molecule comprises the CDRs, variable regions, heavy chains and/or light chains of BAP058-Clone O or BAP058-Clone N disclosed in US 2016/0108123, incorporated by reference in its entirety. Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entireties.

CTLA-4 inhibitors, such as ipilimumab (YERVOY®), tremelimumab, ALPN-202 (Alpine Immune Sciences), RP2 (Replimune), BMS-986249 (Bristol-Myers Squibb), BMS-986218 (Bristol-Myers Squibb), zalifrelimab (Agenus, Ludwig Institute for Cancer Research, UroGen Pharma, Recepta Biopharma), BCD-217 (BIOCAD), Onc-392 (Pfizer, OncoImmune), IBI310 (Innovent Biologics), KN046 (Alphamab), MK-1308 (Merck & Co), REGN4659 (Regeneron Pharmaceuticals), XmAb20717 (Xencor), XmAb22841 (Xencor), Anti-CTLA-4 NF (Bristol-Myers Squibb), MEDI5752 (AstraZeneca), AGEN1181 (Agenus), MGD019 (MacroGenics), ATOR-1015 (Alligator Bioscience), BCD-145 (BIOCAD), PSB205 (Sound Biologics), CS1002 (CStone Pharmaceuticals), ADU-1604 (Aduro Biotech), PF-06753512 (Pfizer), BioInvent-Transgene Research Program (Transgene), AGEN2041 (Agenus, Recepta Biopharam), ATOR-1144 (Alligator Bioscience), CTLA-4 Research Project (Sorrento Therapeutics), PD-L1/CTLA-4 Research Project (Sorrento Therapeutics), HLX13 (Shanghai Henlius Biotech), ISA203 (ISA Pharmaceuticals), PRS-300 Series A (Pieris Pharmaceuticals), BA3071 (BioAtla), CTLA4 Cancer Research Program (Biosortia Pharmaceuticals), RP3 (Replimune), CGO161 (Cold Genesys), APL-509 (Apollomics, JSR), AGEN2041 (Ludwig Institute for Cancer Research), APC 101 (Advanced Proteome), CTLA-4 Inhibitor (Advanced Proteome), BA3071 (BeiGene), BPI-002 (BeyondSpring Pharmaceuticals), CTLA-4 Antibody (Tikcro Technologies), Immuno-Oncology Research Program II (OliPass), PBP1701 (Prestige BioPharma), DB002 (DotBio), DB003 (DotBio), OR-2299 (OncoResponse) and NK044 (Alphamab).

LAG-3 inhibitors, such as LAG525 (Novartis), BMS-986016 (Bristol-Myers Squibb), and TSR-033 (Tesaro). In one embodiment, the LAG-3 inhibitor is an anti-LAG-3 antibody molecule. In one embodiment, the LAG-3 inhibitor is an anti-LAG-3 antibody molecule as disclosed in US 2015/0259420, published on Sep. 17, 2015, entitled “Antibody Molecules to LAG-3 and Uses Thereof,” incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420. In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entireties. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entireties. In one embodiment, the anti-LAG-3 antibody molecule is IMP761 (Prima BioMed). Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entireties. In one embodiment, the anti-LAG-3 inhibitor is a soluble LAG-3 protein, e.g., IMP321 (Prima BioMed), e.g., as disclosed in WO 2009/044273, incorporated by reference in its entirety.

TIM-3 inhibitors, such as MGB453 (Novartis) and TSR-022 (Tesaro). In one embodiment, the TIM-3 inhibitor is an anti-TIM-3 antibody molecule. In one embodiment, the TIM-3 inhibitor is an anti-TIM-3 antibody molecule as disclosed in US 2015/0218274, published on Aug. 6, 2015, entitled “Antibody Molecules to TIM-3 and Uses Thereof,” incorporated by reference in its entirety. In one embodiment, the anti-TIM-3 antibody molecule comprises the CDRs, variable regions, heavy chains and/or light chains of ABTIM3-hum11 or ABTIM3-hum03 disclosed in US 2015/0218274, incorporated by reference in its entirety. In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2. Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entireties.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g., Patents International (e.g., IMS World Publications).

In another aspect of the present disclosure, a kit comprising two or more separate pharmaceutical compositions, at least one of which contains a compound of the present disclosure is provided. In one embodiment, the kit comprises means for separately retaining said compositions, such as a container, divided bottle, or divided foil packet. An example of such a kit is a blister pack, as typically used for the packaging of tablets, capsules and the like.

The kit of the present disclosure may be used for administering different dosage forms, for example, oral and parenteral, for administering the separate compositions at different dosage intervals, or for titrating the separate compositions against one another. To assist compliance, the kit of the present disclosure typically comprises directions for administration, e.g., to treat a disease, disorder or condition described herein.

In the combination therapies of the present disclosure, the compound of the present disclosure and the other therapeutic agent may be manufactured and/or formulated by the same or different manufacturers. Moreover, the compound of the present disclosure and the other therapeutic agent may be brought together into a combination therapy: (i) prior to release of the combination product to physicians (e.g., in the case of a kit comprising the compound of the present disclosure and the other therapeutic agent); (ii) by the physician (or under the guidance of a physician) shortly before administration; (iii) in the patient themselves, e.g., during sequential administration of the compound of the present disclosure and the other therapeutic agent.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition or combination of the present disclosure can be in a unit dosage containing from about 1 to about 1000 mg of active ingredient(s) for a subject of from about 50 to about 70 kg, or from about 1 to about 500 mg, from about 1 to about 250 mg, from about 1 to about 150 mg, from about 0.5 to about 100 mg, or from about 1 to about 50 mg of active ingredient(s) for a subject of from about 50 to about 70 kg. The therapeutically effective dosage of a compound, pharmaceutical composition or pharmaceutical combination is dependent on the species of the subject, the body weight, age and individual condition of the subject, and the disease, disorder or condition or the severity thereof being treated. A physician, clinician or veterinarian of ordinary skill can readily determine the therapeutically effective amount of each of the active ingredients necessary to prevent or treat the progress of the disease, disorder or condition.

The above-cited dosage properties may be demonstrable in in vitro and in vivo tests using advantageously mammals, e.g., mice, rats, dogs, monkeys, or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations. A therapeutically effective amount in vivo may range depending on the route of administration, among other things, between about 0.1 mg/kg to about 500 mg/kg, or between about 1 mg/kg to about 100 mg/kg.

In some embodiments, the concentration of one or more therapeutic agents provided in a pharmaceutical composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.00010% w/w, w/v or v/v.

In some embodiments, the concentration of one or more therapeutic agents provided in a pharmaceutical composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%1, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more therapeutic agents provided in a pharmaceutical composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more therapeutic agents provided in a pharmaceutical composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods of Use

It has now been found that compounds of the present disclosure modulate (e.g., inhibit) one or more PIM kinases, such as PIM1, PIM2 and/or PIM3 kinase, and, therefore, are useful for modulating (e.g., inhibiting) one or more PIM kinases, such as PIM1, PIM2 and/or PIM3 kinase, in vitro or in vivo. Accordingly, provided herein are methods of modulating (e.g., inhibiting) one or more PIM kinases, such as PIM1, PIM2 and/or PIM3 kinase, in a cell (e.g., a cell expressing a PIM kinase), comprising contacting the cell with a compound of the present disclosure (e.g., a compound of Formula I, or a subformula thereof, or a pharmaceutically acceptable salt thereof, such as a therapeutically effective amount of a compound of Formula I, or a subformula thereof, or a pharmaceutically acceptable salt thereof). In some embodiments, the cell is in a subject, such as a human (e.g., a subject having a disease, disorder or condition described herein).

Also provided herein are methods of modulating (e.g., inhibiting) one or more PIM kinases, such as PIM1, PIM2 and/or PIM3 kinase, in a subject, comprising administering to the subject a compound of the present disclosure (e.g., a compound of Formula I, or a subformula thereof, or a pharmaceutically acceptable salt thereof) in an amount effective to inhibit the one or more PIM kinases in the subject.

Also provided herein are methods of modulating (e.g., inhibiting) one or more PIM kinases, such as PIM1, PIM2 and/or PIM3 kinase, in a subject in need thereof (e.g., a subject having a disease, disorder or condition described herein), comprising administering to the subject a therapeutically effective amount of a compound of the present disclosure (e.g., a compound of Formula I, or a subformula thereof, or a pharmaceutically acceptable salt thereof).

A compound of the present disclosure may modulate (e.g., inhibit) all three PIM kinases, or may be selective for one or two PIM kinases (e.g., PIM1 and PIM3 kinases; PIM1 kinase). A compound can be considered to be selective for one PIM kinase (e.g., PIM1 kinase; PIM3 kinase) over another (e.g., PIM2 kinase), when the EC₅₀ and/or IC₅₀ of the compound for a kinase is two times, preferably, five times, more preferably, 10 times, yet more preferably, 20 times, yet more preferably, 50 times, yet more preferably, 100 times, lower than the EC₅₀ and/or IC₅₀, respectively, for another kinase. Methods of measuring EC₅₀ and/or IC₅₀ of compounds of the present disclosure against PIM kinases are described in the Exemplification herein. Other methods for assessing selectivity of a compound are known in the art.

Also provided herein are methods of treating and/or preventing a disease, disorder or condition described herein (e.g., a proliferative disease, disorder or condition, such as cancer; a fibrotic disease, disorder or condition, which is present by itself or comorbid with an infectious, inflammatory or proliferative disease, disorder or condition; an inflammatory disease, disorder or condition) in a subject (e.g., a subject in need thereof). The methods comprise administering to the subject a compound of the present disclosure (e.g., an effective amount, such as a therapeutically effective amount or a prophylactically effective amount, respectively). In certain aspects, the disease, disorder or condition is associated with aberrant expression (e.g., overexpression) of one or more PIM kinases (e.g., PIM1 and PIM2 kinase; PIM1 kinase).

In certain embodiments, the disease, disorder or condition is a proliferative disease. Accordingly, provided herein are methods for treating a proliferative disease in a subject (e.g., a subject in need thereof), comprising administering to the subject a therapeutically effective amount of a compound of the present disclosure.

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: (1) the pathological proliferation of normally quiescent cells; (2) the pathological migration of cells from their normal location (e.g., metastasis of neoplastic cells); (3) the pathological expression of proteolytic enzymes such as the matrix metalloproteinases (e.g., collagenases, gelatinases, and elastases); and/or (4) pathological angiogenesis, as in proliferative retinopathy and tumor metastasis. Non-limiting examples of proliferative diseases include cancer (i.e., “malignant neoplasms”), benign neoplasms, angiogenesis, inflammatory diseases and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending, for example, on the following characteristics: degree of cellular differentiation (including morphology and functionality), rate of growth, local invasion, and metastasis.

A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Non-limiting examples of benign neoplasms include, but are not limited to, lipomas, chondromas, adenomas, acrochordons, senile angiomas, seborrheic keratoses, lentigos, and sebaceous hyperplasias.

In some cases, benign tumors may later give rise to malignant neoplasms, which may result from additional genetic changes in a subpopulation of the tumor's neoplastic cells. Such tumors are referred to as “pre-malignant neoplasms.” An exemplary pre-malignant neoplasm is a teratoma.

A “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue, and is typically identifiable by the presence of a “secondary tumor” or “secondary cell mass” of the tissue type of the primary or original tumor and not of that of the organ or tissue in which the secondary (metastatic) tumor is located.

In certain embodiments, the proliferative disease is cancer. Accordingly, provided herein are methods for treating a cancer in a subject (e.g., a subject in need thereof), comprising administering to the subject a therapeutically effective amount of a compound of the present disclosure.

The term “cancer” refers to a class of diseases characterized by the development of abnormal cells that proliferate uncontrollably and have the ability to infiltrate and destroy normal body tissues.

A wide variety of cancers, including solid tumors, leukemias, lymphomas, and myelomas are amenable to the methods disclosed herein. In some embodiments, the cancer is a solid tumor cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer (e.g., neuroblastoma, glioblastoma, anaplastic astrocytoma), a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer (e.g., anaplastic thyroid cancer), testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma.

In some embodiments, the cancer is a non-solid tumor cancer. In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. Non-limiting examples of hematologic cancers include leukemia (e.g., acute lymphocytic leukemia (ALL) (e.g., B-cell ALL, T-cell ALL), acute myelocytic leukemia (AML) (e.g., B-cell AML, T-cell AML), early T-cell precursor (ETP)-acute lymphoblastic leukemia, chronic myelocytic leukemia (CML) (e.g., B-cell CML, T-cell CML), chronic lymphocytic leukemia (CLL) (e.g., B-cell CLL, T-cell CLL)); lymphoma (e.g., Hodgkin lymphoma (HL) (e.g., B-cell HL, T-cell HL)), non-Hodgkin lymphoma (NHL) (e.g., B-cell NHL such as diffuse large cell lymphoma (DLCL) (e.g., diffuse large B-cell lymphoma)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomads, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma, T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome)), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); heavy chain disease (e.g., alpha chain disease, gamma chain disease, mu chain disease); a myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); multiple myeloma (MM); plasma cell neoplasia; familiar hypereosinophilia; inflammatory myofibroblastic tumors; and immunocytic amyloidosis. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

Examples of cancer treatable according to the methods described herein include, but are not limited to, adenocarcinoma of the breast, prostate, and colon; all forms of bronchogenic carcinoma of the lung; myeloid; melanoma (e.g., metastatic melanoma); astrocytoma (e.g., anaplastic astrocytoma); hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, lung cancer (e.g., large cell lung cancer, such as squamous cell carcinoma, non-small cell lung cancer (NSCLC)), oat cell, papillary, scirrhous, bronchiolar, bronchogenic, squamous cell, and transitional cell). Additional examples of cancer treatable according to the methods described herein include, but are not limited to, histiocytic disorders; leukemia; histiocytosis malignant; Hodgkin's disease; hypereosinophilia, immunoproliferative small; non-Hodgkin's lymphoma; plasmacytoma; reticuloendotheliosis; melanoma (e.g., metastatic melanoma); chondroblastoma; chondroma; chondrosarcoma; fibrotic cancer (e.g., myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans); hepatocellular carcinoma; fibroma; fibrosarcoma; giant cell tumors; histiocytoma; lipoma; liposarcoma; mesothelioma; myxoma; myxosarcoma; osteoma; osteosarcoma; pediatric malignancy, chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatocellular cancer, hepatoma; hidradenoma; islet cell tumor; Leydig cell tumor; papilloma; sertoli cell tumor; theca cell tumor; leiomyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin. Yet more examples of cancer treatable according to the methods described herein include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

HPV-associated cancers are also treatable according to the methods described herein. Non-limiting examples of HPV-associated cancers include cervical cancer, oropharyngeal cancer, anal cancer, vulvar/vaginal cancer, and penile cancer.

Liver cancers, such as hepatocellular cancer (HCC) (e.g., hepatocellular carcinoma, hepatoblastoma, hepatocellular adenoma), malignant hepatoma, hemangiomas and biliary cancer (e.g., cholangiocarcinoma), are also treatable according to the methods described herein.

Musculoskeletal cancers are also treatable according to the methods described herein. Non-limiting examples of musculoskeletal cancers include bone cancer (e.g., osteosarcoma, osteoid osteoma, malignant fibrous histiocytoma, Ewing's sarcoma, chordoma, malignant giant cell tumor chordoma, chondrosarcoma osteochondroma, benign chondroma, chondroblastoma chondromyxofibroma, myelodysplastic syndrome (MDS)), muscle cancer (e.g., rhabdomyosarcoma, rhabdomyoma), connective tissue cancer and synovioma.

Nervous system cancers are also treatable according to the methods described herein. Non-limiting examples of nervous system cancers include brain cancer (e.g., astrocytoma, medulloblastoma, glioma (e.g., astrocytoma, oligodendroglioma), glioblastomas, glioblastoma multiform, medulloblastoma, ependymoma, germinoma (i.e., pinealoma), oligodendroglioma, schwannoma, retinoblastoma, congenital tumors, craniopharyngioma), spinal cord cancer, neurofibroma (e.g., neurofibromatosis (NF) type 1 or type 2, schwannomatosis), neuroblastoma, primitive neuroectodermal tumors (PNT), meningeal cancer (e.g., meningioma, meningiosarcoma, gliomatosis), skull cancer, acoustic neuroma, ependymoma, hemangioblastoma, ocular cancer (e.g., intraocular melanoma, retinoblastoma), pleomorphic xenoanthrocytoma (PXA) and pediatric PXA.

Endocrine/exocrine cancers are also treatable according to the methods described herein. Non-limiting examples of endocrine/exocrine cancers include thyroid cancer (e.g., papillary thyroid carcinoma, follicular thyroid carcinoma, medullary thyroid carcinoma, multiple endocrine neoplasia type 2A, multiple endocrine neoplasia type 2B, familial medullary thyroid cancer, pheochromocytoma, paraganglioma), pancreatic cancer (e.g., pancreatic adenocarcinoma, intraductal papillary mucinous neoplasm (IPMN), islet cell tumors, ductal adenocarcinoma, insulinoma, glucagonoma, vipoma), adrenal gland cancer, neuroendocrine cancer (e.g., gastroenteropancreatic neuroendoctrine tumor (GEP-NET), carcinoid tumor), sebaceous gland carcinoma and sweat gland cancer (e.g., sweat gland carcinoma).

Head and neck cancers, such as squamous cell carcinoma of the head and neck (SCCHN) and adenoid cystic carcinoma, are also treatable according to the methods described herein.

Oral cancers, such as buccal cavity cancer, lip cancer, tongue cancer, mouth cancer, pharynx cancer, hypopharynx cancer (e.g., hypopharyngeal carcinoma), throat cancer (e.g., laryngeal cancer, pharyngeal cancer, nasopharyngeal cancer, oropharyngeal cancer) and salivary gland cancer, are also treatable according to the methods described herein.

Esophageal cancers, such as esophageal squamous cell carcinoma, esophageal adenocarcinoma, Barrett's adenocarcinoma and esophageal leiomyosarcoma, are also treatable according to the methods described herein.

Gastrointestinal cancers are also treatable according to the methods described herein. Non-limiting examples of gastrointestinal cancers include anal cancer, colorectal cancer (e.g., colon cancer, rectal cancer, colorectal adenocarcinoma), gall bladder cancer, gastric cancer (e.g., stomach cancer (e.g., stomach adenocarcinoma)), gastrointestinal stromal tumor (GIST), small bowel cancer (e.g., appendix cancer, small bowel carcinoma, e.g., small bowel adenocarcinoma), small intestine cancer, large bowel cancer and large intestine cancer.

Cardiovascular cancers are also treatable according to the methods described herein. Non-limiting examples of cardiovascular cancers include primary cardiac tumors, angiosarcoma (e.g., lymphangiosarcoma, lymphangioendotheliosarcoma, hemangiosarcoma), endotheliosarcoma (e.g., Kaposi's sarcoma, multiple idiopathic hemorrhagic sarcoma), cardiac myxoma and cardiac rhabdomyoma.

Lung cancers are also treatable according to the methods described herein. Non-limiting examples of lung cancers include bronchus cancer (e.g., bronchogenic carcinoma, bronchial adenoma), alveolar carcinoma, mesothelioma, small cell lung cancer (SCLC), non-small cell lung cancer (NSCLC), lung adenocarcinoma, chondromatous hamartoma and papillary adenocarcinoma.

Genitourinary cancers are also treatable according to the methods described herein. Non-limiting examples of genitourinary cancers include bladder cancer (e.g., urothelial carcinoma), urethral cancer, kidney cancer (e.g., nephroblastoma a.k.a. Wilms' tumor, renal cell carcinoma), testicular cancer (e.g., seminoma, testicular embryonal carcinoma), germ cell cancer, prostate cancer (e.g., prostate adenocarcinoma) and penile cancer (e.g., Paget's disease of the penis and scrotum).

Gynecological cancers are also treatable according to the methods described herein. Non-limiting examples of gynecological cancers include breast cancer (e.g., adenocarcinoma of the breast, papillary carcinoma of the breast, mammary cancer, medullary carcinoma of the breast, triple negative breast cancer, HER-2 positive breast cancer, HER2-negative breast cancer), endometrial cancer (e.g., uterine cancer (e.g., uterine sarcoma, choriocarcinoma), endometrial carcinoma), cervical cancer (e.g., cervical adenocarcinoma), ovarian cancer (e.g., cystadenocarcinoma, ovarian embryonal carcinoma, ovarian adenocarcinoma), germ cell cancer and vulvar cancer (e.g., Paget's disease of the vulva), vaginal cancer and fallopian tube cancer.

Skin cancers are also treatable according to the methods described herein. Non-limiting examples of skin cancers include squamous cell carcinoma (SCC), keratoacanthoma (KA), melanoma, basal cell carcinoma (BCC) and dermatofibroma.

Soft tissue cancers, such as intraepithelial neoplasms, epithelial carcinomas, epithelial sarcomas, adenocarcinomas, adenomas, fibrosarcomas, fibromas, liposarcomas, lipomas, myxomas and teratomas, are also treatable according to the methods described herein.

Myeloproliferative neoplasms are also treatable according to the methods described herein. Non-limiting examples of myeloproliferative neoplasms include myelofibrosis, polycythemia vera and essential thrombocythemia.

Fibrotic cancers are also treatable according to the methods described herein. As used herein, a “fibrotic cancer” is a cancer associated with fibrosis. Fibrosis may precede (e.g., be causative of) or follow (e.g., be caused by) the cancer or treatment of the cancer in fibrotic cancers. Fibrosis may also or alternatively be present with the cancer in fibrotic cancers. Non-limiting examples of fibrotic cancers include myelofibrosis, pancreatic cancer (e.g., pancreatic ductal adenocarcinoma), kidney cancer, liver cancer, lung cancer (e.g., large cell lung cancer, such as squamous cell carcinoma), breast cancer (e.g., inflammatory breast cancer), ovarian cancer (e.g., high grade serious ovarian carcinoma), endometrial cancer, uterine cancer, uterine sarcoma (e.g., uterine leiomyosarcoma), renal cell cancer, sarcoma (e.g., soft tissue sarcoma), malignant fibrous histiocytoma, fibrosarcoma (e.g., dermatofibrosarcoma protuberans), gastric cancer, esophageal cancer, head and neck cancer, cervical cancer, vulvar cancer and hepatocellular cancer (e.g., hepatocellular carcinoma). In some embodiments, the fibrotic cancer is a solid tumor cancer (e.g., kidney, liver, lung, breast, ovarian, endometrial, uterine, and/or pancreatic cancer). In some embodiments, the fibrotic cancer is carcinoma of an internal organ (e.g., pancreas, lung, kidney, liver).

Further examples of cancers treatable according to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Cancer of the anal region; Anal Cancer; Appendix Cancer; Astrocytomas, Childhood; Atypical Teratoid/Rhabdoid Tumor, Childhood, Central Nervous System (CNS); Neoplasms of the CNS (e.g., primary CNS lymphoma, spinal axis tumors, medulloblastoma, brain stem gliomas or pituitary adenomas), Barrett's esophagus (e.g., pre-malignant syndrome), and mycoses fungoides, Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood; Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sezary Syndrome); Ductal Carcinoma In Situ (DCIS); Embryonal Tumors, Central Nervous System, Childhood; Cancer of the Endocrine system (e.g., cancer of the thyroid, pancreas, parathyroid or adrenal glands), Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Gynecologic Tumors ((e.g., uterine sarcomas, carcinoma of the fallopian tubes, carcinoma of the endometrium, carcinoma of the cervix, carcinoma of the vagina or carcinoma of the vulva), Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Cutaneous or Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary; Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma; Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma; Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sezary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis (e.g., renal cell carcinoma, carcinoma of the renal pelvis), benign prostatic hypertrophy, parathyroid cancer, Transitional Cell Cancer; Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. Accordingly, in some embodiments, the cancer is a metastatic cancer. In other embodiments, the cancer is a pre-metastatic cancer.

In certain embodiments, the cancer is a rare cancer. The term “rare cancer” refers to cancers that occur in a relatively small number of patients.

In some embodiments, a proliferative disease, such as cancer (e.g., a fibrotic cancer), is treated by targeting a tumor stromal cell (e.g., in a tumor microenvironment), such as a cancer-associated fibroblast (CAF), stellate cell or myofibroblast, and/or an immune cell, such as a tumor-associated immune cell (e.g., in the tumor-immune microenvironment), for example, to thereby modulate the tumor-stroma microenvironment and/or the tumor-immune microenvironment.

Cachexia is linked to chronic illness and manifests in involuntary weight loss (e.g., greater than 5% of pre-illness weight) resulting from the atrophy of skeletal muscle and adipose tissues. This condition is distinct from other conditions, like anorexia, where fat stores are depleted but muscle mass remains largely intact. Cachexia affects over half of cancer patients resulting in poor quality of life (fatigue and weakness) and can sometimes even compromise treatment strategies in some individuals. Myostatin, a transforming growth factor-beta (TGF-beta) super-family member, has been well characterized as a negative regulator of muscle growth and development. Without wishing to be bound by any particular theory, it is believed that blocking this pathway would potentially benefit cancer patients, specifically patients with late stage disease and metastasis where cachexia is prominent. Thus, in some embodiments, the disease or condition is cachexia (e.g. cancer cachexia).

In some embodiments, the disease, disorder or condition is a fibrotic disease, disorder or condition. As used herein, the terms “fibrosis” and “fibrotic disease, disorder or condition” are used interchangeably, and refer to a disease, disorder or condition in a subject involving the formation of excess fibrous connective tissue in an organ or tissue. The formation of excess fibrous connective tissue leading to a fibrosis is believed to occur in an organ or tissue in a reparative or reactive process. This can be a reactive, benign, or pathological state. Physiologically, fibrosis acts to deposit connective tissue, which can interfere with, or totally inhibit the normal architecture and function of an underlying organ or tissue. For example, pulmonary fibrosis is a respiratory disease in which scars are formed in the lung tissues, leading to serious breathing problems. Scar formation typically involves the accumulation of excess fibrous connective tissue, and often leads to thickening of the walls and causes reduced oxygen supply in the blood. Reduced oxygen supply in the blood, in turn, can lead to heart failure, and even death. The replacement of normal lung with scar tissue causes a decrease in oxygen diffusion capacity. Some types of pulmonary fibrosis are believed to be perpetuated by aberrant wound healing, rather than chronic inflammation. Once the scarring has developed, it often remains, even after treatment with current therapies. Idiopathic pulmonary fibrosis (IPF) is a type of pulmonary fibrosis, and is a fatal lung disease with an unknown etiology that can be present with inflammation, cancer, and/or viral infection.

In pulmonary fibrosis, the fibrotic process is commonly considered the result of a recurrent injury to the alveolar epithelium followed by an uncontrolled proliferation of fibroblasts. In general, fibrosis progresses in three stages (illustrated for pulmonary fibrosis, but common across many fibrotic conditions): the injury stage (“Stage 1”), the epithelial-fibroblastic interaction stage (“Stage 2”), and the aberrant repair and fibrosis stage (“Stage 3”). In Stage 1, generally, the epithelium is damaged, and one or more of the following events can occur: epithelial damage, endothelial damage, for example, in pulmonary fibrosis, destruction of an alveolar capillary basilar membrane, vascular leak, platelet activation, and fibrin clot activation. In Stage 2, generally, fibroblasts begin to interact with the damaged epithelium, and one or more of the following events can occur: release of profibrotic cytokines, (myo)fibroblast recruitment, proliferation, and differentiation, provisional matrix formation, angiogenesis, and defective re-epithelialization. In Stage 3, generally, the epithelial damage is aberrantly repaired resulting in fibrosis, and one or more of the following events can occur: exaggerated extracellular matrix (ECM) accumulation, lack of matrix degradation, for example, in pulmonary fibrosis, progressive lung remodeling and honeycomb changes (in pulmonary fibrosis, the lung tissue comes to resemble a honeycomb).

Non-limiting examples of fibrotic diseases, disorders and conditions include cancer-associated fibrosis; lung fibrosis, commonly known as “scarring of the lungs” (e.g., pulmonary fibrosis, for example, idiopathic pulmonary fibrosis, acute exacerbation of idiopathic pulmonary fibrosis or familial pulmonary fibrosis); liver fibrosis (hepatic fibrosis, e.g., keloids, scleroderma, nephrogenic systemic fibrosis, bile duct fibrosis (biliary fibrosis), liver cirrhosis, for example, primary biliary cholangitis (biliary cirrhosis), primary sclerosing cholangitis); cardiac disease; cardiac fibrosis or restenosis (e.g., in-stent restenosis, post-angioplasty restenosis); vascular fibrosis; kidney fibrosis (renal fibrosis); skin fibrosis (cutaneous fibrosis or endometrial fibrosis, e.g., keloids, scleroderma, or nephrogenic systemic fibrosis); gastrointestinal fibrosis (e.g., Crohn's disease); bone marrow fibrosis (myelofibrosis); athrofibrosis (e.g., of the knee, the shoulder or another joint); Dupuytren's contracture; mediastinal fibrosis; Peyronie's disease; retroperitoneal fibrosis; systemic sclerosis; autoimmune hepatitis; nonalcoholic steatohepatitis; cystic fibrosis; beta-thalassemia; actinic keratosis; hypertension; chronic kidney disease; Chagas' heart disease; dry eye; ulcer; corneal fibrosis; wet age-related macular degeneration; chronic wound (failure to heal, close); psoriasis. In some embodiments, the fibrotic disease, disorder or condition is lung fibrosis, for example, pulmonary fibrosis, such as idiopathic pulmonary fibrosis, acute exacerbation of idiopathic pulmonary fibrosis or familial pulmonary fibrosis. In some embodiments, the fibrotic disease, disorder or condition is liver fibrosis. In some embodiments, the fibrotic disease, disorder or condition is a cardiac disease, or cardiac fibrosis or restenosis, for example, in-stent restenosis, post-angioplasty restenosis. In some embodiments, the fibrotic disease, disorder or condition is cardiac fibrosis or restenosis.

Fibrosis may be associated with another disease, disorder or condition (e.g., inflammation, an inflammatory disease, disorder or condition, a proliferative disease, such as cancer, a viral or bacterial infection or the like) or may occur independently. For example, fibrosis may precede (e.g., be causative of) or follow (e.g., be caused by) another disease, disorder or condition. Fibrosis may also or alternatively be present, whether associated or not, with another disease, disorder or condition (e.g., inflammation, an inflammatory disease, disorder or condition, a proliferative disease, such as cancer, a viral or bacterial infection or the like), or may be present without a concomitant disease, disorder or condition (e.g., associated disease, disorder or condition). In some embodiments, the fibrosis is present without an associated disease, disorder or condition. In some embodiments, the fibrosis is present with an associated disease, disorder or condition.

Although the occurrence of fibrosis associated with another disease, disorder or condition is not uncommon, for example, the presence of cancer-associated fibrosis, the etiology of fibrosis is not well understood and fibrosis occurs also independently from and/or in the absence of other diseases, disorders or conditions. However, it is believed that similar mechanisms and signaling pathways are present in both fibrosis and many associated diseases, disorders or conditions affecting organs or tissues in which fibrosis is also present, for example, the presence of IPF with lung cancer. For example, it is believed that fibrosis along with many diseases with which it is often present, progress via the TGFβ protein and the signaling cascade implicated by overexpression of it, see for example, Ballester, B; et al., Idiopathic Pulmonary Fibrosis and lung Cancer: Mechanisms and Molecular targets, Int. J. Mol. Sci. 2019, 20, 593; doi:10.3390/ijms20030593.

Fibrosis can be comorbid with, caused by and/or exacerbated by an associated disease, disorder or condition (e.g., an infection, such as an infection described herein, such as a viral or bacterial infection; an inflammatory disease, disorder or condition, such as an inflammatory disease, disorder or condition described herein; or a proliferative disease, such as a proliferative disease described herein, such as cancer, in particular, fibrotic cancer). Thus, in some embodiments, a disease, disorder or condition associated with fibrosis is a comorbid, causative and/or exacerbating disease, disorder or condition. In some embodiments, the fibrosis is comorbid with the associated disease, disorder or condition. For example, fibrosis can be comorbid with an infection, for example, a viral or bacterial infection; an inflammatory disease, disorder or condition, such as an inflammatory disease, disorder or condition described herein; or a proliferative disease, such as a proliferative disease described herein, such as cancer, in particular, fibrotic cancer. In some embodiments, the fibrosis is caused by the associated disease, disorder or condition (e.g., the fibrosis is caused by an infection, for example, a viral or bacterial infection; an inflammatory disease, disorder or condition, such as an inflammatory disease, disorder or condition described herein; or a proliferative disease, such as a proliferative disease described herein, such as cancer). In some embodiments, the fibrosis is comorbid with and/or caused by the associated disease, disorder or condition (e.g., an infection, for example, a viral or bacterial infection; an inflammatory disease, disorder or condition, such as an inflammatory disease, disorder or condition described herein; or a proliferative disease, such as a proliferative disease described herein, such as cancer, in particular, fibrotic cancer). In some embodiments, the fibrosis is exacerbated by the associated disease, disorder or condition. For example, fibrosis can be exacerbated by an infection, for example, a viral or bacterial infection; an inflammatory disease, disorder or condition, such as an inflammatory disease, disorder or condition described herein; or a proliferative disease, such as a proliferative disease described herein, such as cancer, in particular, fibrotic cancer.

In some embodiments, the disease, disorder or condition associated with fibrosis is an infection (e.g., viral infection, bacterial infection). In further embodiments, the infection is a viral infection (concomitant with a viral infection). Non-limiting examples of viral infections include Orthomyxoviridae viral infection (e.g., an influenza A viral infection or an influenza B viral infection), a Pneumoviridae viral infection (e.g., a metapneumovirus viral infection, such as human metapneumovirus (HMPV) infection, or an orthopneumovirus infection, such as respiratory syncytial virus (RSV) (e.g., human respiratory syncytial virus (HRSV) infection, such as human respiratory syncytial virus A2 (HRSV-A2) infection or human respiratory syncytial virus B1 (HRSV—B1) infection)), a Orthohepadnavirus viral infection (e.g., a Hepatitis B viral infection), a Hepacivirus viral infection (e.g., a Hepatitis C virus infection), a Paramyxoviridae viral infection (e.g., a Respirovirus infection, such as a human parainfluenza virus type 1 (HPIV-1) infection or a human parainfluenza type 3 (HPIV-3) infection, or a Rubulavirus viral infection, such as a human parainfluenza virus type 2 (HPIV-2) infection or a human parainfluenza type 4 (HPIV-4) infection), an Adenoviridae viral infection (e.g., a Mastadenovirus infection, such as a human adenovirus B (HAdV-B) infection or a human adenovirus C (HAdV-C) infection) and an Enterovirus viral infection (e.g., a Rhinovirus A infection, a Rhinovirus B infection or a Rhinovirus C infection). The infection associated with fibrosis can be a comorbid, causative and/or exacerbating infection.

In some embodiments, the disease, disorder or condition associated with fibrosis is an inflammatory disease, disorder or condition.

As used herein, “inflammatory disease, disorder or condition” refers to disease, disorder or condition involving the response of one or more of a subject's body tissues to stimuli recognized as harmful by the body. Non-limiting examples of inflammatory diseases, disorders or conditions include non-alcoholic fatty liver disease (NAFLD), alcoholic steatohepatitis (ASH), non-alcoholic steatohepatitis (NASH), primary biliary cholangitis (PBC), primary sclerosing cholangitis, autoimmune hepatitis, skin inflammation and psoriasis. In some embodiments, an inflammatory disease, disorder or condition is non-alcoholic fatty liver disease (NAFLD), alcoholic steatohepatitis (ASH), non-alcoholic steatohepatitis (NASH), primary biliary cholangitis (PBC), primary sclerosing cholangitis, autoimmune hepatitis, skin inflammation or psoriasis.

The inflammatory disease, disorder or condition associated with fibrosis can be a comorbid, causative and/or exacerbating disease, disorder or condition.

In some embodiments, an inflammatory disease, disorder or condition is an autoimmune disease, disorder or condition, such as osteoarthritis, rheumatoid arthritis, pain, inflammatory bowel disease, a respiratory disorder or a skin disorder. In some embodiments, the inflammatory disease, disorder or condition is an inflammatory bowel disease, e.g., Crohn's disease, ulcerative colitis or irritable bowel syndrome. In some embodiments, the inflammatory disease, disorder or condition is a respiratory disorder, e.g., asthma, rhinitis, chronic obstructive pulmonary disease, bronchitis, nasal polyposis, nasal congestion, farmer's lung fibroid lung or cough. In some embodiments, the inflammatory disease, disorder or condition is a skin disorder, e.g., dermatitis, cutaneous eosinophilias, Lichen planus, urticaria, psoriasis, pruritus, angiodermas, corneal ulcer, chronic skin ulcer, conjunctivitis, vaculitides, uveitis or erythema.

In some embodiments, the disease, disorder or condition associated with fibrosis is a cancer, such as any of the cancers described herein, in particular, a fibrotic cancer. Stated otherwise, in some embodiments, the fibrosis is cancer-associated fibrosis. The cancer can be a comorbid, causative and/or exacerbating cancer. Alternatively, in some embodiments, the fibrosis is not associated with a cancer.

It will be appreciated that some fibroses can be associated with cancer (e.g., fibrotic cancer), but can also occur independently from and/or in the absence of the associated cancer. For example, IPF can be associated with lung cancer, but can also occur independently from and/or in the absence of lung cancer. Accordingly, in some embodiments, the fibrosis is present in the absence of cancer (e.g., a fibrotic cancer), for example, IPF is present in the absence of lung cancer.

Some embodiments comprise identifying a subject who has fibrosis or who is at risk of developing a fibrosis (e.g., due to an associated disease, disorder or condition, such as a comorbid, causative, or exacerbating disease, disorder or condition), and administering to the subject an effective amount (e.g., a therapeutically effective amount, prophylactically effective amount) of a compound of the present disclosure.

Administration of a compound of the present disclosure, alone or in combination with one or more additional therapeutic agents, including any of those described herein, can occur during a single stage of fibrosis (e.g., Stage 1, Stage 2, Stage 3), or can be divided across multiple stages of fibrosis (e.g., two stages, three stages). For example, a compound of the present disclosure can be administered during Stage 1, Stage 2 or Stage 3 of fibrosis, while one or more additional therapeutic agents can be administered during a different stage of the fibrosis. Alternatively, a compound of the present disclosure and one or more additional therapeutic agent(s) can be administered during all stages of the fibrosis.

In various embodiments, an amount effective to treat a fibrotic disease, disorder or condition is an amount effective to slow down or stop the progression of the fibrotic disease, disorder or condition, increase the survival time of a subject suffering with the fibrotic disease, disorder or condition (e.g., by at least 10%, 20%, 30%, 40%, 50%, 60%, 70%, 80%, 90%, or 100%, when compared with a subject not administered the therapy), increase the survival rate in a subject population (e.g., survival after being admitted to the intensive care unit increases by at least 10%, 20%, 30%, 40%, 50%, 60%, 70%, 80%, 90%, or 100% when compared with a subject population that was not administered the therapy), reduce the risk of a subject developing the fibrotic disease, disorder or condition when compared with a subject that was not administered the therapy, preserve organ function (e.g., lung function, liver function) when compared with a subject that was not administered the therapy, and/or prevent or reduce the risk of acute exacerbation of the fibrotic disease, disorder or condition when compared with a subject that was not administered the therapy.

Also provided herein are methods of inhibiting fibrosis in a tissue, comprising contacting the tissue (e.g., in vitro, ex vivo, in vivo) with a compound of the present disclosure (e.g., an effective amount of a compound of the present disclosure). In various embodiments, an effective amount is an amount effective to inhibit the formation or deposition of tissue fibrosis, and/or reduce the size, cellularity, composition, cellular or collagen content of a fibrotic lesion. In some embodiments, the tissue is in a subject (e.g., a human).

Also provided herein are methods for targeting a tumor stromal cell or immune cell (e.g., tumor-associated immune cell), and/or (e.g., and thereby) modulating (e.g., normalizing) tumor microenvironment (e.g., tumor-stroma microenvironment and/or tumor-immune microenvironment) in vivo or in vitro, the methods comprising contacting a tumor stromal cell or an immune cell (e.g., a tumor-associated immune cell) with a compound of the present disclosure. In certain embodiments, the inhibition occurs in vivo in a subject. In certain embodiments, the inhibition occurs in vitro (e.g., in a cell line, tissue or biological sample). In certain embodiments, the tumor stromal cell is a cancer-associated fibroblast (CAF), a stellate cell or a myofibroblast.

Without wishing to be bound by any particular theory, it is believed that certain compounds can normalize the tumor microenvironment and thereby improve blood vessel perfusion and drug delivery. Enhanced drug delivery is expected, in turn, to enhance the efficacy of a drug, such as an immunomodulator (e.g., immunooncology agent), including any immunomodulator described herein. Accordingly, also provided herein are methods for modulating (e.g., normalizing) tumor microenvironment (e.g., tumor-stroma microenvironment and/or tumor-immune microenvironment) in vivo or in vitro, the methods comprising contacting a tumor with a compound of the present disclosure.

Also provided herein are methods of inhibiting viral infection and/or viral replication in a subject in need thereof, comprising administering to the subject an effective amount (e.g., a therapeutically effective amount, prophylactically effective amount) of a compound of the present disclosure.

Also provided herein are methods for treating and/or preventing an inflammatory disease, disorder or condition (e.g., any of the inflammatory diseases, disorders or conditions described herein) in a subject in need thereof, comprising administering to the subject an effective amount (e.g., a therapeutically effective amount, prophylactically effective amount, respectively) of a compound of the present disclosure.

The compounds of the present disclosure can be administered as a monotherapy, or can be administered as part of a combination therapy, as described herein, with other therapeutic agents and/or treatment modalities. Accordingly, in some embodiments, the methods described herein further comprise administering to the subject one or more additional therapies (e.g., one or more additional therapeutic agents). The compound of the present disclosure and the additional therapy(ies) can be co-administered, e.g., in a simultaneous or substantially simultaneous manner. The compound of the present disclosure and the additional therapy(ies) can also or alternatively be administered sequentially, either at approximately the same time or at different times. For example, the compound of the present disclosure can be administered before the additional therapy(ies). Or, the compound of the present disclosure can be administered after the additional therapy(ies). Suitable additional therapies for use in the methods disclosed herein include those discussed herein in the context of combinations.

Therapeutic agents (e.g., compounds of the present disclosure) and pharmaceutical compositions thereof can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen. In some embodiments, a therapeutic agent (e.g., a compound of the present disclosure) is administered orally. In some embodiments, a therapeutic agent (e.g., compound of the present disclosure) is administered intravenously.

### EXEMPLIFICATION

The compounds of the present disclosure can be prepared in a number of ways known to one skilled in the art of organic synthesis in view of the methods, reaction schemes and examples provided herein. The compounds of the present disclosure can be synthesized using the methods described below, together with synthetic methods known in the art of synthetic organic chemistry, or by variations thereon, as appreciated by those skilled in the art. Preferred methods include, but are not limited to, those described below. The reactions are performed in a solvent or solvent mixture appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

The starting materials are generally available from commercial sources such as Sigma Aldrich or other commercial vendors, or are prepared as described in this disclosure, or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), Larock, R. C., Comprehensive Organic Transformations, 2^{nd} ed., Wiley-VCH Weinheim, Germany (1999), or Beilsteins Handbuch der organischen Chemie, 4, Aufl. ed. Springer-Verlag, Berlin, including supplements (also available via the Beilstein online database).

For illustrative purposes, the reaction schemes depicted below provide potential routes for synthesizing the compounds of the present disclosure as well as key intermediates. Those skilled in the art will appreciate that other synthetic routes may be used to synthesize the compounds of the present disclosure. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in view of this disclosure using conventional chemistry well known to those skilled in the art.

In the preparation of compounds of the present disclosure, protection of remote functionality of intermediates may be necessary. The need for such protection will vary depending on the nature of the remote functionality and the conditions of the preparation methods. The need for such protection is readily determined by one skilled in the art. For a general description of protecting groups and their use, see Greene, T. W. et al., Protecting Groups in Organic Synthesis, 4th Ed., Wiley (2007). Protecting groups incorporated in making of the compounds of the present disclosure, such as the trityl protecting group, may be shown as one regioisomer but may also exist as a mixture of regioisomers.

### Example 1. Synthetic Experimentals

### 3-bromo-6-chloroimidazo[1,2-b]pyridazine

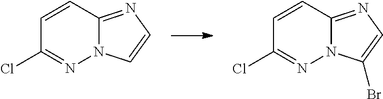 [SMILES: Clc1ccc2nccn2n1 ; Clc1ccc2ncc(Br)n2n1]

To an ice-cold solution of 6-chloroimidazo[1,2-b]pyridazine (100 g, 651 mmol) in acetonitrile (750 ml) under stirring N-bromosuccinimide (NBS, 116 g, 651 mmol) was cautiously added portionwise. The resulting mixture was stirred at room temperature for 3 hours (h). After confirming the completion of reaction by thin layer chromatography (TLC), reaction was quenched with slow addition of ice-cold water. Then reaction mixture was extracted with ethyl acetate (EtOAc, 3×5000 mL). Combined organic layers were washed with saturated bicarbonate solution (3×5000 mL) and brine solution (3000 mL), then dried over sodium sulfate, filtered and evaporated under reduced pressure to afford the title compound, which was used in the next step without further purification. LCMS (ESI positive ion) m/z: 234.1 (M+2).

### Compound 1: 4-((3-(5-ethyl-1H-pyrrol-3-yl)imidazo[1,2-b]pyridazin-6-yl)amino)bicyclo[2.2.1]heptan-1-ol

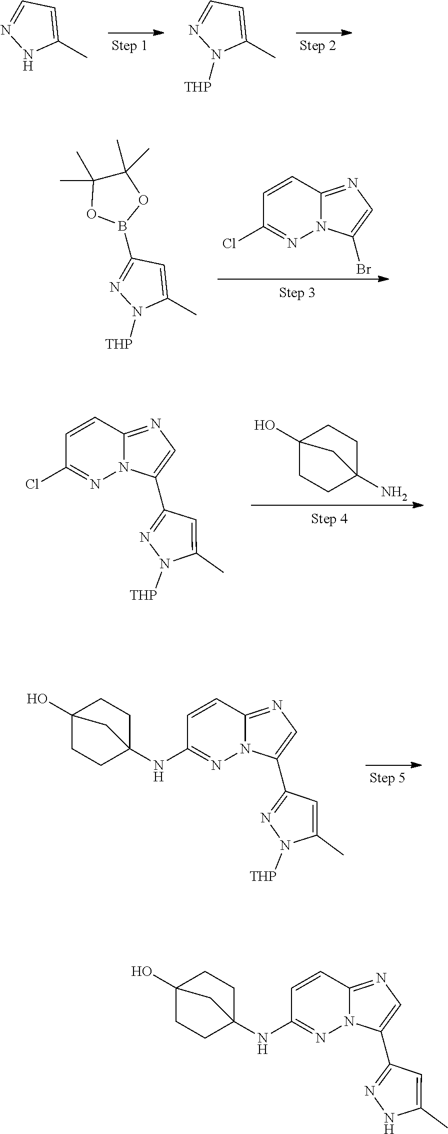

Step 1: 5-methyl-1-(tetrahydro-2H-pyran-2-yl)-1H-pyrazole. A round-bottomed flask was charged with 5-methyl-1H-pyrazole (4 g, 48.7 mmol) in 3,4-dihydro-2H-pyran (12.29 g, 146 mmol). To this suspension trifluoroacetic acid (0.263 ml, 3.41 mmol) was added. The clear solution was heated to 75° C. for 18 h. The reaction mixture was diluted with EtOAc (100 mL), and the organic phase was washed with aqueous saturated NaHCO₃ (2×30 mL), water (20 mL) and brine (20 mL), then dried over Na₂SO₄, filtered and concentrated under reduced pressure. The residue thus obtained was purified by flash chromatography (18% EtOAc in petroleum ether) to give the title compound. LCMS (ESI positive ion) m/z: 167.1 (M+1). ¹H-NMR (400 MHz, CDCl₃): δ 7.50 (d, J=2.40 Hz, 1H), 6.05-6.09 (m, 1H), 5.26-5.41 (m, 1H), 4.07-4.11 (m, 1H), 3.67-3.72 (m, 1H), 2.36 (s, 3H), 2.01-2.13 (m, 3H), 1.58-1.73 (m, 3H).

Step 2: 5-methyl-1-(tetrahydro-2H-pyran-2-yl)-3-(4,4,5,5-tetramethyl-1,3,2-dioxaborolan-2-yl)-1H-pyrazole. A round-bottomed flask was charged with 5-methyl-1-(tetrahydro-2H-pyran-2-yl)-1H-pyrazole (1.0 g, 6.02 mmol) in tetrahydrofuran (THF, 15 mL), and flask was maintained at −78° C. To this solution n-butyl lithium (n-BuLi, 3.95 mL, 6.32 mmol) was added slowly over a period of 10 minutes (min). The resulting reaction mixture was allowed to stir for a period of 15 min. To the reaction mixture triisopropyl borate (1.537 mL, 6.62 mmol) was added dropwise at −78° C. The reaction mixture was stirred for 15 min, at which time, the reaction mixture was allowed to reach to room temperature, and pinacol (0.782 g, 6.62 mmol) was added followed by acetic acid (0.689 mL, 12.03 mmol). The reaction mixture was stirred at room temperature overnight. The reaction mixture was diluted with heptane and the organic phase was washed with NH₄Cl (aqueous (aq)), NaHCO₃ (aq) and brine, then dried over Na₂SO₄, filtered and concentrated under reduced pressure to obtain the title compound, which was used in the next step without purification. LCMS (ESI positive ion) m/z: 211.1 (M+1).

Step 3: 6-chloro-3-(5-methyl-1-(tetrahydro-2H-pyran-2-yl)-1H-pyrazol-3-yl)imidazo[1,2-b]pyridazine. A sealed tube was charged with 3-bromo-6-chloroimidazo[1,2-b]pyridazine (250 mg, 1.075 mmol), 5-methyl-1-(tetrahydro-2H-pyran-2-yl)-3-(4,4,5,5-tetramethyl-1,3,2-dioxaborolan-2-yl)-1H-pyrazole (377 mg, 1.291 mmol) and K₂CO₃ (372 mg, 2.69 mmol) in 1,4-dioxane (4 mL) and water (1 mL). Nitrogen was purged through the solution for 5 minutes, then PdCl₂(dppf)·DCM (88 mg, 0.108 mmol) was added, and the reaction mixture was again purged with nitrogen for 5 minutes. The reaction mixture was kept in oil bath and allowed to stir at 100° C. for 16 h. The progress of the reaction was monitored by TLC and liquid chromatography-mass spectrometry (LCMS). The reaction was diluted with water (15 mL) and extracted with ethyl acetate (25 mL×3). The combined organic layers were washed with brine (20 mL), dried over anhydrous sodium sulfate, filtered and concentrated under reduced pressure to afford the crude product, which was purified by flash silica chromatography using 60% ethyl acetate in petroleum ether as eluent to obtain the title compound. LCMS (ESI positive ion) m/z: 318.1 (M+1). ¹H-NMR (400 MHz, DMSO-d6): δ 8.35 (d, J=12.8 Hz, 1H), 8.00 (s, 1H), 7.47-7.69 (m, 1H), 6.66 (s, 1H), 5.27 (d, J=10.8 Hz, 1H), 3.90-3.94 (m, 2H), 3.55-3.62 (m, 2H), 2.38 (s, 3H), 1.78-1.94 (m, 2H), 1.45-1.62 (m, 2H).

Step 4: 4-((3-(5-methyl-1-(tetrahydro-2H-pyran-2-yl)-1H-pyrazol-3-yl)imidazo[1,2-b]pyridazin-6-yl)amino)bicyclo[2.2.1]heptan-1-ol. A sealed tube was charged with 6-chloro-3-(5-methyl-1-(tetrahydro-2H-pyran-2-yl)-1H-pyrazol-3-yl)imidazo[1,2-b]pyridazine (150 mg, 0.472 mmol), 4-aminobicyclo[2.2.1]heptan-1-ol·HCl (60.0 mg, 0.472 mmol) and potassium fluoride (82 mg, 1.416 mmol) in dimethylsulfoxide (DMSO, 1.5 ml). The reaction mixture was stirred in oil bath at 120° C. for 16 h and completion of the reaction was monitored by TLC and LCMS. Reaction was diluted with water (10 mL) and product was extracted with EtOAc (3×20 mL). Combined organic layers were washed with brine, dried over Na₂SO₄, filtered and concentrated under reduced pressure. The residue thus obtained was purified by flash silica chromatography using 5% methanol (MeOH) in dichloromethane (DCM) as eluent to obtain the title compound. LCMS (ESI positive ion) m/z: 409.2 (M+1).

Step 5: 4-((3-(5-methyl-1H-pyrazol-3-yl)imidazo[1,2-b]pyridazin-6-yl)amino)bicyclo[2.2.1]heptan-1-ol. To an ice-cold solution of 4-((3-(5-methyl-1-(tetrahydro-2H-pyran-2-yl)-1H-pyrazol-3-yl)imidazo[1,2-b]pyridazin-6-yl)amino)bicyclo[2.2.1]heptan-1-ol (123 mg, 0.301 mmol) in DCM (2 mL) was added 4M HCl in dioxane (3 ml, 12.00 mmol). The reaction mixture was allowed to stir at 25° C. for 16 h and completion of the reaction was monitored by TLC. Reaction was concentrated and basified with methanolic ammonia and again concentrated to obtain the crude product. The residue thus obtained was purified by flash chromatography using 8% methanol in DCM as eluent to yield the title compound. LCMS (ESI positive ion) m/z: 325.2 (M+1); HPLC: 96.05%. ¹H-NMR (400 MHz, MeOD): δ 7.78 (s, 1H), 7.59-7.61 (m, 1H), 6.98 (s, 1H), 6.60-6.70 (m, 1H), 2.10-2.44 (m, 5H), 2.10 (s, 2H), 1.81-1.92 (m, 6H).

### Compound 2: 2-(4-((3-(5-methyl-1H-pyrazol-3-yl)imidazo[1,2-b]pyridazin-6-yl)amino)bicyclo[2.2.2]octan-1-yl)propan-2-ol

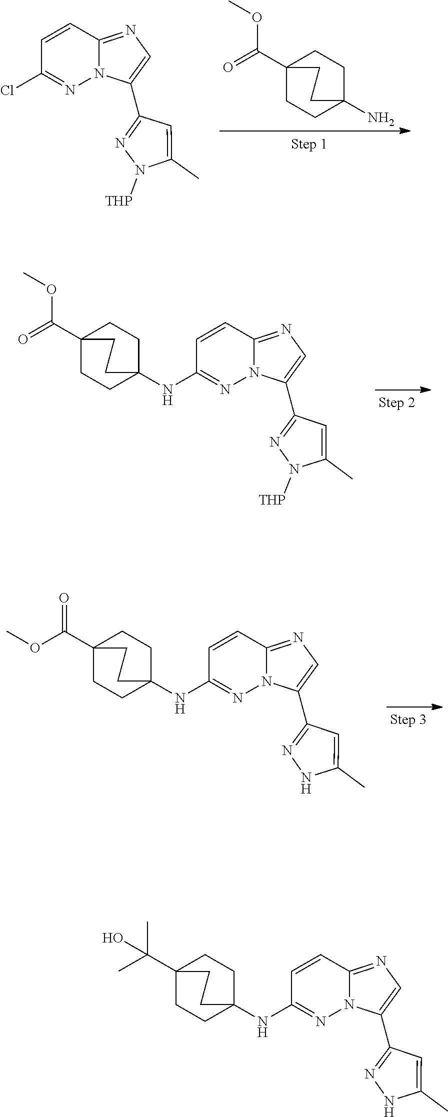

Step 1: methyl 4-((3-(5-methyl-1-(tetrahydro-2H-pyran-2-yl)-1H-pyrazol-3-yl)imidazo[1,2-b]pyridazin-6-yl)amino)bicyclo[2.2.2]octane-1-carboxylate. A sealed tube was charged with 6-chloro-3-(5-methyl-1-(tetrahydro-2H-pyran-2-yl)-1H-pyrazol-3-yl)imidazo[1,2-b]pyridazine (300 mg, 0.944 mmol), methyl 4-aminobicyclo[2.2.2]octane-1-carboxylate (173 mg, 0.944 mmol) and potassium fluoride (82 mg, 1.416 mmol) in DMSO (3 mL). The reaction mixture was stirred in oil bath at 120° C. for 30 h and completion of the reaction was monitored by TLC and LCMS. Reaction was diluted with water (25 mL) and product was extracted with EtOAc (3×20 mL). Combined organic layers were washed with brine, dried over Na₂SO₄, filtered and concentrated under reduced pressure. The residue thus obtained was purified by flash silica chromatography using 4% MeOH in DCM as eluent to obtain the title compound. LCMS (ESI positive ion) m/z: 465.3 (M+1). 6-Chloro-3-(5-methyl-1-(tetrahydro-2H-pyran-2-yl)-1H-pyrazol-3-yl)imidazo[1,2-b]pyridazine was made in accordance with Steps 1-3 in the synthesis of Compound 1.

Step 2: methyl 4-((3-(5-methyl-1H-pyrazol-3-yl)imidazo[1,2-b]pyridazin-6-yl)amino)bicyclo[2.2.2]octane-1-carboxylate. To an ice-cold solution of 4-((3-(5-methyl-1-(tetrahydro-2H-pyran-2-yl)-1H-pyrazol-3-yl)imidazo[1,2-b]pyridazin-6-yl)amino)bicyclo[2.2.2]octane-1-carboxylate (145 mg, 0.312 mmol) in DCM (2 mL) was added 4M HCl in dioxane (3 mL). The reaction mixture was allowed to stir at 25° C. for 16 h and completion of the reaction was monitored by TLC. Reaction was quenched with NaHCO₃ (25 mL) and product was extracted with ethyl acetate (3×20 mL). The combined organic layers were washed with brine, dried over Na₂SO₄, filtered and concentrated under reduced pressure to obtain the crude product (100 mg), which was used in the next step without further purification. LCMS (ESI positive ion) m/z: 381.2 (M+1).

Step 3: 2-(4-((3-(5-methyl-1H-pyrazol-3-yl)imidazo[1,2-b]pyridazin-6-yl)amino)bicyclo[2.2.2]octan-1-yl)propan-2-ol. A round-bottomed flask was charged with methyl 4-((3-(5-methyl-1H-pyrazol-3-yl)imidazo[1,2-b]pyridazin-6-yl)amino)bicyclo[2.2.2]octane-1-carboxylate (100 mg, 0.263 mmol) in THE (8 mL) maintained under nitrogen atmosphere. A solution of MeMgBr (1.4M THF:Toluene) (3.75 mL, 5.26 mmol) was added slowly to above reaction mixture at 25° C. The reaction mixture was allowed to stir at 65° C. for 6 h, and completion of the reaction was monitored by TLC. The reaction mixture was quenched with NH₄Cl (25 mL), and product was extracted with ethyl acetate (3×20 mL). The combined organic layers were washed with brine, dried over Na₂SO₄, filtered and concentrated under reduced pressure. The residue thus obtained was purified by preparative high-pressure liquid chromatography (HPLC) to obtain the title compound. LCMS (ESI positive ion) m/z: 381.2 (M+1), HPLC: 99.47%. ¹H-NMR (400 MHz, DMSO-d6): δ 7.65-7.72 (m, 2H), 6.92 (s, 1H), 6.63-6.66 (m, 1H), 3.93 (s, 2H), 2.29 (s, 2H), 1.90-2.03 (m, 8H), 1.61-1.65 (m, 6H), 1.04 (s, 6H).

### Compound 3: 4-((3-(5-ethyl-1H-pyrrol-3-yl)imidazo[1,2-b]pyridazin-6-yl)amino)bicyclo[2.2.1]heptan-1-ol

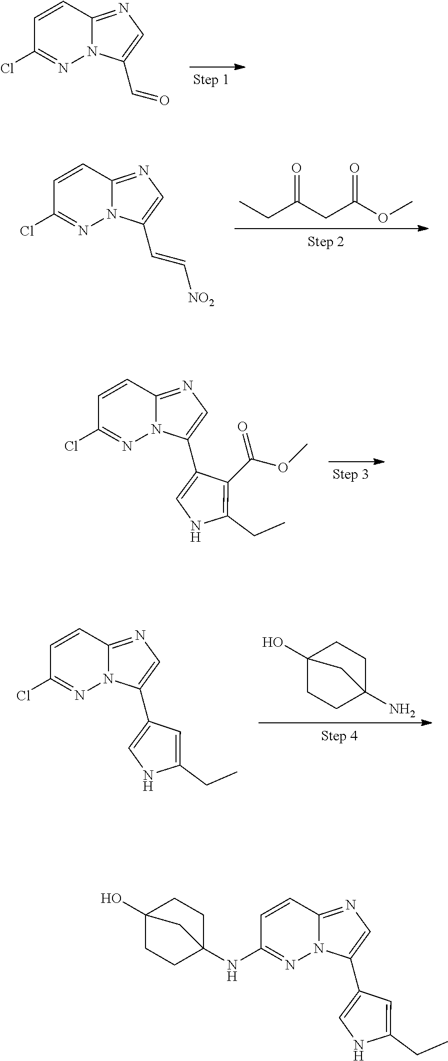

Step 1: (E)-6-chloro-3-(2-nitrovinyl)imidazo[1,2-b]pyridazine. A round-bottomed flask was charged with 6-chloroimidazo[1,2-b]pyridazine-3-carbaldehyde (10 g, 55.1 mmol), ammonium acetate (12.74 g, 165 mmol) and nitromethane (16.81 g, 275 mmol) in acetic acid (30 mL). The reaction mixture was allowed to stir at 80° C. for 2 h, and completion of the reaction was monitored by TLC. Reaction mixture was diluted with water (150 mL) and product was extracted with EtOAc (3×70 mL). Combined organic layers were washed with brine, dried over Na₂SO₄, filtered and concentrated under reduced pressure to obtain the crude product, which was used in the next step without purification. LCMS (ESI positive ion) m/z: calculated: 225.0 (M+1).

Step 2: methyl 4-(6-chloroimidazo[1,2-b]pyridazin-3-yl)-2-ethyl-1H-pyrrole-3-carboxylate. To a solution of (E)-6-chloro-3-(2-nitrovinyl)imidazo[1,2-b]pyridazine (6 g, 26.7 mmol) and methyl 3-oxopentanoate (5.21 g, 40.1 mmol) in MeOH (150 mL) was added sodium methoxide (0.722 g, 13.36 mmol). The reaction mixture was allowed to stir at room temperature for 30 minutes, then methanolic ammonia (7M) (19.08 mL, 134 mmol) was added, and the reaction mixture was stirred at room temperature for 16 h. The completion of the reaction was monitored by TLC. The organic solvent was removed under reduced pressure, and the residue was diluted with water (100 mL). The aqueous layer was extracted with EtOAc (3×100 mL). The combined organic layers were washed with brine, dried over Na₂SO₄, filtered and concentrated. The residue thus obtained was purified by flash silica chromatography using 70% EtOAc in petroleum ether as eluent to obtain the title compound. LCMS (ESI positive ion) m/z: 305.0 (M+1). ¹H-NMR (400 MHz, DMSO-d6): δ 11.62 (brs, 1H), 8.18-8.22 (m, 1H), 7.84-7.88 (d, 1H), 7.28-7.32 (m, 1H), 7.09 (d, 1H), 3.38-3.58 (m, 3H), 2.88-2.94 (m, 4H), 1.2 (t, 3H).

Step 3: 6-chloro-3-(5-ethyl-1H-pyrrol-3-yl)imidazo[1,2-b]pyridazine. A mixture of methyl 4-(6-chloroimidazo[1,2-b]pyridazin-3-yl)-2-ethyl-1H-pyrrole-3-carboxylate (600 mg, 1.969 mmol) in 10 N HCl (1 mL, 10.00 mmol) was allowed to stir at 100° C. under microwave radiation for 2 h. The completion of the reaction was monitored by TLC. Reaction was diluted with water (10 mL), and the precipitate obtained was filtered and washed with water. Solid cake was dried under vacuum to obtain the title compound. LCMS (ESI positive ion) m/z: calculated: 247.1 (M+1).

Step 4: 4-((3-(5-ethyl-1H-pyrrol-3-yl)imidazo[1,2-b]pyridazin-6-yl)amino)bicyclo[2.2.1]heptan-1-ol. A mixture of 6-chloro-3-(5-ethyl-1H-pyrrol-3-yl)imidazo[1,2-b]pyridazine (100 mg, 0.405 mmol), 4-aminobicyclo[2.2.1]heptan-1-ol (103 mg, 0.811 mmol) and sodium tert-butoxide (78 mg, 0.811 mmol) in 1,4-dioxane (3 mL) was purged with nitrogen for 10 minutes. To this solution Pd₂(dba)₃ (18.56 mg, 0.020 mmol) and 2-(dicyclohexylphosphino)-3,6-dimethoxy-2′,4′,6′-triisopropyl-1,1′-biphenyl (21.76 mg, 0.041 mmol) was added. The resulting reaction mixture was again purged with nitrogen for 5 minutes. The reaction mixture was allowed to stir at 100° C. under microwave radiation for 2 h. The completion of the reaction was monitored by TLC. Reaction mixture was filtered through Celite, and washed with 10% MeOH in DCM. The organic layer was concentrated under reduced pressure. The residue thus obtained was purified by flash silica chromatography using 6% MeOH in DCM as eluent to obtain the title compound. LCMS (ESI positive ion) m/z: calculated: 338.2 (M+1). HPLC: 96.2%. ¹H-NMR (400 MHz, DMSO-d6): δ 10.81 (s, 1H), 7.60 (t, J=9.60 Hz, 1H), 7.48 (d, J=24.40 Hz, 1H), 6.94 (s, 1H), 6.66 (d, J=9.60 Hz, 1H), 6.54 (s, 1H), 6.34 (d, J=Hz, 1H), 4.98 (s, 1H), 3.50 (s, 1H), 3.17-3.18 (m, 1H), 2.59 (d, J=7.60 Hz, 2H), 2.25-2.31 (m, 2H), 1.74-1.84 (m, 3H), 1.62-1.66 (m, 3H), 1.19-1.30 (m, 3H).

### Compound 4: 2-(4-((3-(5-cyclopropyl-1H-pyrazol-3-yl)imidazo[1,2-b]pyridazin-6-yl)amino)bicyclo[2.2.2]octan-1-yl)propan-2-ol

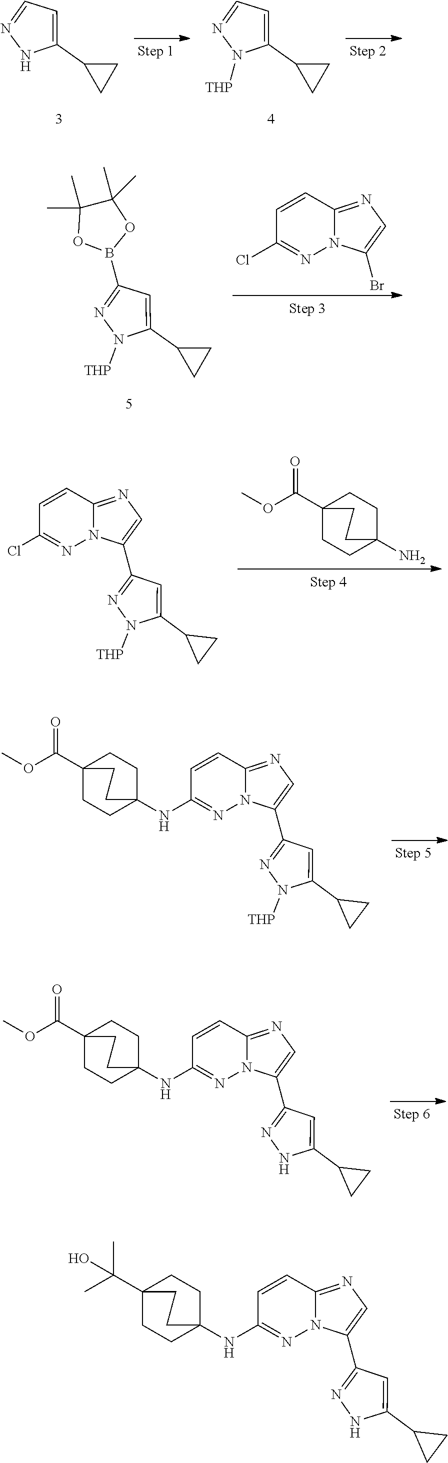

Step 1: Preparation of 5-cyclopropyl-1-(tetrahydro-2H-pyran-2-yl)-1H-pyrazole. A mixture of 5-cyclopropyl-1H-pyrazole (50 g, 462 mmol), para-toluene sulfonic acid (PTSOH, 4.40 g, 23.12 mmol), 3,4-dihydro-2H-pyran (DHP, 63.4 ml, 694 mmol) in EtOAc (100 mL) was stirred at 80° C. for 2 h. The completion of reaction was monitored by TLC. The reaction mixture was diluted with EtOAc and the organic phase was washed with 10% aqueous NaHCO₃ solution (3×500 mL) and brine. The combined organic layers were dried over Na₂SO₄, filtered and concentrated under reduced pressure. The residue thus obtained was purified by flash column chromatography (silica gel, 230-400 mesh size) using 10% EtOAc in petroleum ether as an eluent to afford the title compound. LCMS (ESI positive ion) m/z: 193.2 (M+1).

Step 2: Preparation of 5-cyclopropyl-1-(tetrahydro-2H-pyran-2-yl)-3-(4,4,5,5-tetramethyl-1,3,2-dioxaborolan-2-yl)-1H-pyrazole. In a round-bottomed flask, a solution of 5-cyclopropyl-1-(tetrahydro-2H-pyran-2-yl)-1H-pyrazole (26 g, 135 mmol) in THE (200 ml) was maintained at −78° C. To this solution, n-BuLi (2.5 M in hexane, 81 mL, 203 mmol) was added over a period of 10 minutes. After 20 minutes, 2-isopropoxy-4,4,5,5-tetramethyl-1,3,2-dioxaborolane (33.1 mL, 162 mmol) was added dropwise at −78° C., and the reaction mixture was stirred for additional 15 minutes. The reaction mixture was allowed to reach room temperature and was stirred for 8 h. The reaction mixture was quenched with aqueous NH₄Cl solution (100 mL), and extracted with ethyl acetate (3×100 mL). The combined organic layers were washed with brine, filtered, dried over Na₂SO₄ and concentrated to afford the title compound. Crude was taken to the next step without further purification. LCMS (ESI positive ion) m/z: 319.2 (M+1).

Step 3: Preparation of 6-chloro-3-(5-cyclopropyl-1-(tetrahydro-2H-pyran-2-yl)-1H-pyrazol-3-yl)imidazo[1,2-b]pyridazine. To a stirred solution of 3-bromo-6-chloroimidazo[1,2-b]pyridazine (14 g, 60.2 mmol) and 5-cyclopropyl-1-(tetrahydro-2H-pyran-2-yl)-3-(4,4,5,5-tetramethyl-1,3,2-dioxaborolan-2-yl)-1H-pyrazole (42.2 g, 66.2 mmol) in 1,4-dioxane:water (5:1, 120 mL) was added K₂CO₃ (16.65 g, 120 mmol). The solution was degassed under nitrogen atmosphere for 15 minutes, then PdCl₂(dppf)-DCM adduct (4.92 g, 6.02 mmol) was added to the reaction mixture. The resulting mixture was stirred at 100° C. for 16 h, and the completion of reaction was monitored by TLC. The mixture was filtered through a pad of Celite, washed with ethyl acetate, and the filtrate was concentrated under reduced pressure. The residue thus obtained was purified by flash column chromatography (silica gel, 230-400 mesh size) using 80% EtOAc in petroleum ether as an eluent to afford the title compound. LCMS (ESI positive ion) m/z: calculated: 344.1 (M+1).

Step 4: Preparation of methyl 4-((3-(5-cyclopropyl-1-(tetrahydro-2H-pyran-2-yl)-1H-pyrazol-3-yl)imidazo[1,2-b]pyridazin-6-yl)amino)bicyclo[2.2.2]octane-1-carboxylate. To a stirred solution of 6-chloro-3-(5-cyclopropyl-1-(tetrahydro-2H-pyran-2-yl)-1H-pyrazol-3-yl)imidazo[1,2-b]pyridazine (11 g, 26.6 mmol) and methyl 4-aminobicyclo[2.2.2]octane-1-carboxylate (5.60 g, 30.5 mmol) in DMSO (100 mL) was added potassium fluoride (9.26 g, 159 mmol). The resulting mixture was stirred at 120° C. for 18 h. After confirming the completion of reaction by LCMS, reaction was quenched with ice-cold water. The quenched reaction mixture was extracted with ethyl acetate (3×250 mL). Combined organic layers were washed with brine, dried over Na₂SO₄, filtered and concentrated under reduced pressure. The crude product was purified by flash column chromatography (silica gel, 230-400 mesh size) using 80% EtOAc in petroleum ether as an eluent to obtain the title compound. LCMS (ESI positive ion) m/z: 491.3 (M+1). ¹H-NMR (400 MHz, DMSO-d6): δ 7.74 (d, J=9.60 Hz, 1H), 7.57 (s, 1H), 6.73 (t, J=8.00 Hz, 2H), 6.57 (s, 1H), 5.30-5.33 (m, 1H), 3.60 (s, 3H), 2.42-2.46 (m, 2H), 1.94-2.01 (m, 8H), 1.84-1.88 (m, 8H), 1.56-1.65 (m, 3H), 0.93-0.97 (m, 2H) and 0.66-0.69 (m, 2H).

Step 5: Preparation of methyl 4-((3-(5-cyclopropyl-1H-pyrazol-3-yl)imidazo[1,2-b]pyridazin-6-yl)amino)bicyclo[2.2.2]octane-1-carboxylate. To a stirred solution of methyl 4-((3-(5-cyclopropyl-1-(tetrahydro-2H-pyran-2-yl)-1H-pyrazol-3-yl)imidazo[1,2-b]pyridazin-6-yl)amino)bicyclo[2.2.2]octane-1-carboxylate (6.9 g, 14.06 mmol) in DCM (30 mL) was added 4M HCl in dioxane (30 mL, 120 mmol) at room temperature. The resulting mixture was stirred at room temperature for 18 h. After confirming the completion of reaction by LCMS, the solvent was evaporated under reduced pressure to obtain crude residue. The crude residue was dissolved in water, basified with NaHCO₃ solution and extracted with ethyl acetate (3×100 mL). Combined organic layers were washed with brine (200 mL), dried over Na₂SO₄, filtered and evaporated under reduced pressure to afford the crude product. The crude product was used in the next step without further purification. LCMS (ESI positive ion) m/z: 407.2 (M+1).

Step 6: Preparation of 2-(4-((3-(5-cyclopropyl-1H-pyrazol-3-yl)imidazo[1,2-b]pyridazin-6-yl)amino)bicyclo[2.2.2]octan-1-yl)propan-2-ol. To a stirred solution of methyl 4-((3-(5-cyclopropyl-1H-pyrazol-3-yl)imidazo[1,2-b]pyridazin-6-yl)amino)bicyclo[2.2.2]octane-1-carboxylate (5 g, 10.95 mmol) in dry THE (100 mL) was added methylmagnesium bromide (73.0 ml, 219 mmol) in a dropwise manner at −10° C. The resulting mixture was stirred at room temperature for 3 h. After confirming the completion of reaction by LCMS, the reaction was quenched with saturated NH₄Cl solution (20 mL), and product was extracted with EtOAc (3×50 mL). Combined organic layers were dried over Na₂SO₄, filtered and concentrated under reduced pressure to get crude product. The crude product was purified by flash column chromatography (silica gel, 230-400 mesh size) using 5% methanol in dichloromethane as an eluent to afford the title compound. LCMS (ESI positive ion) m/z: 407.2 (M+1). HPLC purity; 97.64%. ¹H-NMR (400 MHz, DMSO-d6): δ 12.85 (s, 1H), 7.63-7.69 (m, 2H), 6.55-6.70 (m, 3H), 3.93 (s, 1H), 1.97-2.00 (m, 7H), 1.64 (brs, 6H), 0.93-1.06 (m, 8H), 0.65-0.71 (m, 2H).

### Compound 5: 4-((3-(5-cyclopropyl-1H-pyrazol-3-yl)imidazo[1,2-b]pyridazin-6-yl)amino)bicyclo[2.2.1]heptan-1-ol

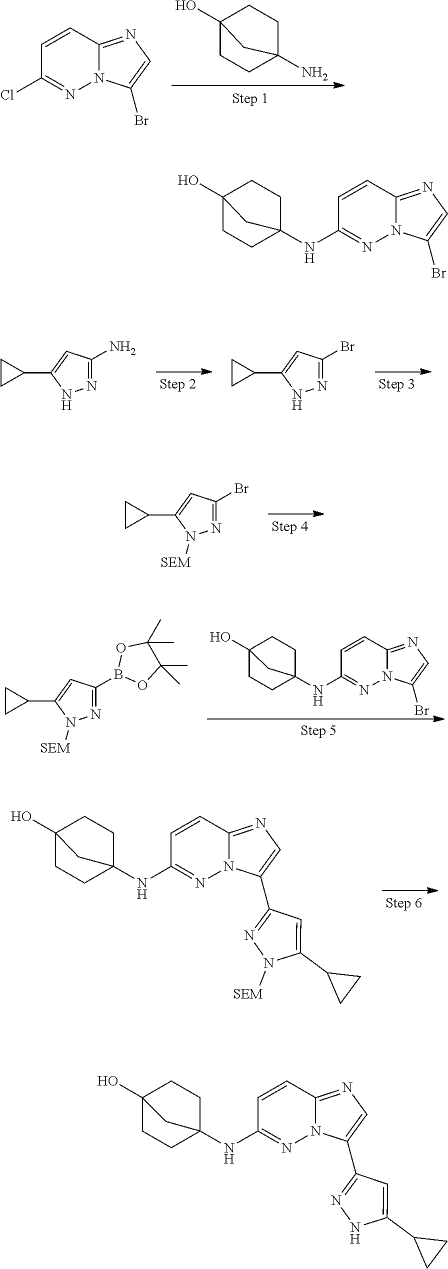

Step 1: Preparation of 4-((3-bromoimidazo[1,2-b]pyridazin-6-yl)amino)bicyclo[2.2.1]heptan-1-ol. To a stirred solution of 3-bromo-6-chloroimidazo[1,2-b]pyridazine (200 mg, 0.860 mmol) and 4-aminobicyclo[2.2.1]heptan-1-ol hydrochloride (141 mg, 0.860 mmol) in DMSO (5 mL) was added potassium fluoride (150 mg, 2.58 mmol). The resulting mixture was stirred at 120° C. for 18 h. After confirming the completion of reaction by LCMS, reaction was quenched with slow addition of ice-cold water. The quenched reaction mixture was extracted with EtOAc (3×50 mL). Combined organic layers were washed with brine (10 mL), dried over Na₂SO₄, filtered and evaporated under reduced pressure to afford the title compound, which was used in the next step without further purification. LCMS (ESI positive ion) m/z: 325 (M+2).

Step 2: Preparation of 3-bromo-5-cyclopropyl-1H-pyrazole. To an ice-cold, stirred solution of 5-cyclopropyl-1H-pyrazol-3-amine (10 g, 81 mmol), copper(II) bromide (8.16 g, 36.5 mmol) and copper(I) bromide (0.582 g, 4.06 mmol) in acetonitrile (150 mL), tert-butyl nitrite (14.47 ml, 122 mmol) was cautiously added portionwise. The resulting mixture was stirred at 0° C. for 3 h. After confirming the completion of reaction by LCMS, reaction was quenched with sodium bicarbonate solution. The quenched reaction mixture was extracted with ethyl acetate (3×500 mL). Combined organic layers were filtered through a bed of Celite, and washed with ethyl acetate. Organic layer was washed with brine (300 mL), dried over Na₂SO₄, filtered and evaporated under reduced pressure to afford the title compound, which was used in the next step without further purification. LCMS (ESI positive ion) m/z: calculated: 189 (M+2).

Step 3: Preparation of 3-bromo-5-cyclopropyl-1-((2-(trimethylsilyl)ethoxy)methyl)-1H-pyrazole. To an ice-cold suspension of NaH (2.57 g, 64.2 mmol) in THE (50 ml), 3-bromo-5-cyclopropyl-1H-pyrazole (8 g, 42.8 mmol) was added at 0° C. The reaction mixture was stirred at the same temperature for 1.5 hour and subsequently 2-(trimethylsilyl)ethoxymethyl chloride (8.34 ml, 47.0 mmol) was added. The resulting mixture was stirred at 0° C. for additional 1.5 h. After confirming the completion of reaction by TLC, reaction mixture was quenched with ice water (100 mL) and extracted with ethyl acetate (3×100 mL). The combined organic layers were washed with brine (200 mL), dried over anhydrous Na₂SO₄, filtered and concentrated under reduced pressure to afford crude product. The crude product was purified by column chromatography using 2% EtOAc in petroleum ether as eluent to afford the title compound. LCMS (ESI positive ion) m/z: calculated: 319 (M+2).

Step 4: Preparation of 5-cyclopropyl-3-(4,4,5,5-tetramethyl-1,3,2-dioxaborolan-2-yl)-1-((2-(trimethylsilyl)ethoxy)methyl)-1H-pyrazole. To a solution of 3-bromo-5-cyclopropyl-1-((2-(trimethylsilyl)ethoxy)methyl)-1H-pyrazole (4 g, 12.61 mmol) and 2-isopropoxy-4,4,5,5-tetramethyl-1,3,2-dioxaborolane (5.14 ml, 25.2 mmol) in THE (50 ml) maintained at −78° C., nBuLi (12.10 mL, 30.3 mmol) was added slowly over a period of 10 minutes. The resulting reaction mixture was brought to room temperature, and stirred for 6 h. After confirming the completion of reaction by TLC, the reaction mixture was quenched with saturated ammonium chloride solution and extracted with ethyl acetate (3×60 mL). The combined organic layers were washed with brine (100 mL), dried over anhydrous Na₂SO₄, filtered and concentrated under reduced pressure to afford the title compound, which was used in the next step without further purification. LCMS (ESI positive ion) m/z: calculated: 365.1 (M+1). NOTE: The product was a mixture of boronic acid and boronic ester.

Step 5: Preparation of 4-((3-(5-cyclopropyl-1-((2-(trimethylsilyl)ethoxy)methyl)-1H-pyrazol-3-yl)imidazo[1,2-b]pyridazin-6-yl)amino)bicyclo[2.2.1]heptan-1-ol. A stirred solution of 4-((3-bromoimidazo[1,2-b]pyridazin-6-yl)amino)bicyclo[2.2.1]heptan-1-ol (200 mg, 0.495 mmol; synthesized in the first step), 5-cyclopropyl-3-(4,4,5,5-tetramethyl-1,3,2-dioxaborolan-2-yl)-1-((2-(trimethylsilyl)ethoxy)methyl)-1H-pyrazole (313 mg, 0.644 mmol), Na₂CO₃ (79 mg, 0.743 mmol) and Cs₂CO₃ (113 mg, 0.347 mmol) in dioxane:water (3:1, 4 mL) was purged with nitrogen for 10 minutes. To this solution was added PdCl₂(dppf)-DCM adduct (40.4 mg, 0.050 mmol), and the reaction mixture was again purged with nitrogen for 10 minutes. The resulting mixture was stirred under microwave radiation at 120° C. for 2 h. After confirming the completion of reaction by TLC, the reaction mixture was filtered through a pad of Celite, washed with EtOAc and concentrated under reduced pressure to afford the crude product. The crude product was purified by flash column chromatography (silica gel, 230-400 mesh size) using 10% MeOH in DCM as an eluent to obtain the title compound. LCMS (ESI positive ion) m/z: calculated: 481.3 (M+1).

Step 6: Preparation of 4-((3-(5-cyclopropyl-1H-pyrazol-3-yl)imidazo[1,2-b]pyridazin-6-yl)amino)bicyclo[2.2.1]heptan-1-ol. To a stirred solution of 4-((3-(5-cyclopropyl-1-((2-(trimethylsilyl)ethoxy)methyl)-1H-pyrazol-3-yl)imidazo[1,2-b]pyridazin-6-yl)amino)bicyclo[2.2.1]heptan-1-ol (400 mg, 0.416 mmol) in DCM (3 mL), trifluoroacetic acid (TFA, 0.032 ml, 0.416 mmol) was added to the reaction mixture at room temperature. The resulting mixture was stirred at room temperature for 18 h. After confirming the completion of reaction by LCMS, the reaction mixture was concentrated under reduced pressure. The crude residue thus obtained was purified by reverse-phase, preparative HPLC (eluting with aqueous 0.1% TFA-acetonitrile) to afford the TFA salt of the title compound. LCMS (ESI positive ion) m/z: 351 (M+1). HPLC purity: 99.11%. ¹H-NMR (400 MHz, DMSO-d6): δ 8.18 (s, 1H), 7.96 (d, J=9.60 Hz, 1H), 7.76 (s, 1H), 7.08 (d, J=9.20 Hz, 1H), 6.72 (s, 1H), 2.15-2.21 (m, 2H), 2.03-2.09 (m, 1H), 1.90 (brs, 4H), 1.81-1.86 (m, 2H), 1.63-1.78 (m, 2H), 1.00-1.05 (m, 2H) and 0.74-0.78 (m, 2H).

### Compound 6: 2-(4-((3-(5-ethyl-1H-pyrazol-3-yl)imidazo[1,2-b]pyridazin-6-yl)amino)bicyclo[2.2.2]octan-1-yl)propan-2-ol

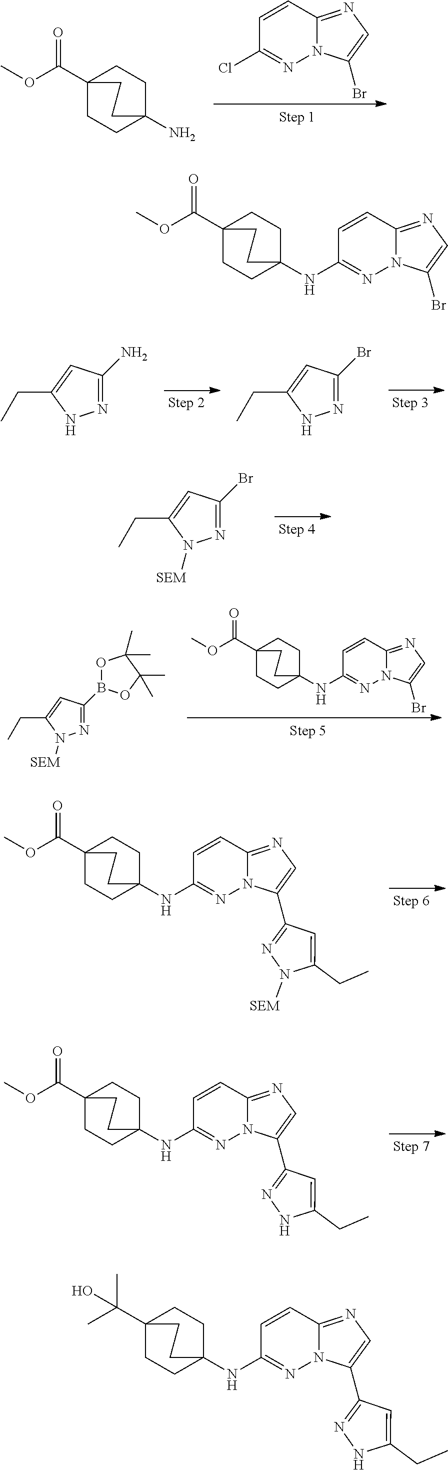

Step 1: Preparation of methyl 4-((3-bromoimidazo[1,2-b]pyridazin-6-yl)amino)bicyclo[2.2.2]octane-1-carboxylate. To a stirred solution of 3-bromo-6-chloroimidazo[1,2-b]pyridazine (1 g, 4.30 mmol) and methyl 4-aminobicyclo[2.2.2]octane-1-carboxylate (0.788 g, 4.30 mmol) in DMSO (10 mL) was added potassium fluoride (1.250 g, 21.51 mmol). The resulting mixture was stirred at 120° C. for 18 h. After confirming the completion of reaction by LCMS, reaction was quenched with ice-cold water. The aqueous layer was extracted with EtOAc (3×50 mL). Combined organic layers were washed with brine (100 mL), dried over Na₂SO₄, filtered and evaporated under reduced pressure. The crude residue thus obtained was purified by flash column chromatography (silica gel, 230-400 mesh size) using 80% EtOAc in petroleum ether as an eluent to afford the title compound. LCMS (ESI positive ion) m/z: 381 (M+2).

Step 2: Preparation of 4-((3-(5-cyclopropyl-1-((2-(trimethylsilyl)ethoxy)methyl)-1H-pyrazol-3-yl)imidazo[1,2-b]pyridazin-6-yl)amino)bicyclo[2.2.1]heptan-1-ol. To an ice-cold stirred solution of 5-ethyl-1H-pyrazol-3-amine (10 g, 90 mmol), copper(II) bromide (9.04 g, 40.5 mmol) and copper(I) bromide (0.645 g, 4.50 mmol) in acetonitrile (100 mL), tert-butyl nitrite (16.16 ml, 135 mmol) was cautiously added portionwise. The resulting mixture was stirred at 0° C. for 2 h. After confirming the completion of reaction by LCMS, reaction was quenched with sodium bicarbonate solution. The quenched reaction mixture was extracted with EtOAc (3×500 mL). Combined organic layers were filtered through a bed of Celite, and washed with excess EtOAc. Organic layer was washed with brine (300 mL), dried over anhydrous Na₂SO₄, filtered and concentrated under reduced pressure to afford the title compound, which was used in the next step without further purification. LCMS (ESI positive ion) m/z: 177.1 (M+2).

Step 3: Preparation of 3-bromo-5-ethyl-1-((2-(trimethylsilyl)ethoxy)methyl)-1H-pyrazole. To an ice-cold suspension of sodium hydride (4.11 g, 103 mmol) in THE (100 mL) was added 3-bromo-5-ethyl-1H-pyrazole (12 g, 68.6 mmol) at 0° C. The mixture was stirred at the same temperature for 1.5 h. To the mixture SEM-Cl (12.57 g, 75 mmol) was slowly added at 0° C. The resulting reaction mixture was stirred at 0° C. for another 1.5 h. After confirming the completion of reaction by TLC, the reaction mixture was quenched with ice water (100 mL) and extracted with ethyl acetate (3×100 mL). The combined organic layers were washed with brine (200 mL), dried over anhydrous Na₂SO₄, filtered and concentrated under reduced pressure. The crude residue thus obtained was purified by column chromatography using 2% EtOAc in petroleum ether as eluent to afford the title compound. LCMS (ESI positive ion) m/z: calculated: 306.9 (M+2).

Step 4: Preparation of 5-ethyl-3-(4,4,5,5-tetramethyl-1,3,2-dioxaborolan-2-yl)-1-((2-(trimethylsilyl)ethoxy)methyl)-1H-pyrazole. To an ice-cold, stirred solution 3-bromo-5-ethyl-1-((2-(trimethylsilyl)ethoxy)methyl)-1H-pyrazole (3.7 g, 12.12 mmol) and 2-isopropoxy-4,4,5,5-tetramethyl-1,3,2-dioxaborolane (4.51 g, 24.24 mmol) in THE (50 mL) maintained at −78° C., nBuLi (11.63 ml, 29.1 mmol) was added slowly over a period of 15 minutes. The resulting mixture was stirred at −78° C. for 2 h. After confirming the completion of reaction by TLC, the reaction mixture was quenched with saturated ammonium chloride solution and extracted with ethyl acetate (3×50 mL). The combined organic layers were washed with brine (200 mL), dried over anhydrous Na₂SO₄, filtered and concentrated under reduced pressure to afford the title compound, which was used in the next step without further purification. LCMS (ESI positive ion) m/z: 353.2 (M+1). NOTE: Crude product was a mixture of boronic acid and boronic ester.

Step 5: Preparation of methyl 4-((3-(5-ethyl-1-((2-(trimethylsilyl)ethoxy)methyl)-1H-pyrazol-3-yl)imidazo[1,2-b]pyridazin-6-yl)amino)bicyclo[2.2.2]octane-1-carboxylate. A stirred solution of methyl 4-((3-bromoimidazo[1,2-b]pyridazin-6-yl)amino)bicyclo[2.2.2]octane-1-carboxylate (700 mg, 1.513 mmol), 5-ethyl-3-(4,4,5,5-tetramethyl-1,3,2-dioxaborolan-2-yl)-1-((2-(trimethylsilyl)ethoxy)methyl)-1H-pyrazole (990 mg, 1.968 mmol), Na₂CO₃ (241 mg, 2.270 mmol) and Cs₂CO₃ (345 mg, 1.059 mmol) in dioxane:water (3:1, 4 mL) was purged with nitrogen for 15 minutes. To this mixture, PdCl₂(dppf)-CH₂Cl₂ adduct (124 mg, 0.151 mmol) was added, and the resulting mixture was again purged with nitrogen for 15 minutes. The resulting mixture was stirred under microwave radiation at 120° C. for 2 h. After confirming the completion of reaction by LCMS, the reaction mixture was filtered through a pad of Celite, and washed with excess EtOAc. The organic layer was concentrated under reduced pressure to afford crude. The crude product was purified by flash column chromatography (silica gel, 230-400 mesh size) using 5% MeOH in DCM as an eluent to give the title compound. LCMS (ESI positive ion) m/z: 525.3 (M+1).

Step 6: Preparation of methyl 4-((3-(5-ethyl-1H-pyrazol-3-yl)imidazo[1,2-b]pyridazin-6-yl)amino)bicyclo[2.2.2]octane-1-carboxylate. To a stirred solution of methyl 4-((3-(5-ethyl-1-((2-(trimethylsilyl)ethoxy)methyl)-1H-pyrazol-3-yl)imidazo[1,2-b]pyridazin-6-yl)amino)bicyclo[2.2.2]octane-1-carboxylate (380 mg, 0.724 mmol) in DCM (10 mL) TFA (0.558 mL, 7.24 mmol) was added at room temperature. The resulting mixture was stirred at room temperature for 18 h. After confirming the completion of reaction by TLC, the reaction mixture was concentrated under reduced pressure. The residue was dissolved in water and basified with sodium bicarbonate solution, and extracted with EtOAc. The crude product was purified by flash column chromatography (silica gel, 230-400 mesh size) using 5% MeOH in DCM as an eluent to obtain the title compound. LCMS (ESI positive ion) m/z: 395.3 (M+1). ¹H-NMR (400 MHz, DMSO-d6): δ 12.76 (s, 1H), 7.69 (br s, 1H), 7.69 (s, 1H), 6.65 (brs, 2H), 3.60 (s, 3H), 2.57-2.79 (m, 2H), 2.07-2.09 (m, 6H), 1.89-1.96 (m, 6H) and 1.28 (t, J=9.60 Hz, 3H).

Step 7: Preparation of 2-(4-((3-(5-ethyl-1H-pyrazol-3-yl)imidazo[1,2-b]pyridazin-6-yl)amino)bicyclo[2.2.2]octan-1-yl)propan-2-ol. Methyl magnesium bromide (4.22 ml, 12.67 mmol) was added to a stirred solution of methyl 4-((3-(5-ethyl-1H-pyrazol-3-yl)imidazo[1,2-b]pyridazin-6-yl)amino)bicyclo[2.2.2]octane-1-carboxylate (250 mg, 0.634 mmol) in THF (10 ml) at 0° C. The reaction mixture was heated to 65° C., and stirred at 65° C. for 2 h. After confirming the completion of reaction by TLC and LCMS, the reaction was quenched with saturated NH₄Cl (20 mL) and extracted with EtOAc (3×25 mL). Combined organic layers were dried over Na₂SO₄, filtered and concentrated under reduced pressure. The crude residue thus obtained was purified by flash column chromatography (silica gel, 230-400 mesh size) using 5% MeOH in DCM as an eluent to afford the title compound. LCMS (ESI positive ion) m/z: 395.3 (M+1). HPLC purity: 96.34%. ¹H-NMR (400 MHz, DMSO-d6): δ 7.65 (t, J=10.40 Hz, 2H), 6.94 (s, 1H), 6.55-6.70 (m, 2H), 3.91 (s, 1H), 2.67-2.72 (m, 2H), 2.01 (br s, 6H), 1.63 (br s, 6H), 1.29 (t, J=7.60 Hz, 3H), 1.03 (s, 6H).

### Compound 7: 4-((3-(5-ethyl-1H-pyrazol-3-yl)imidazo[1,2-b]pyridazin-6-yl)amino)bicyclo[2.2.1]heptan-1-ol

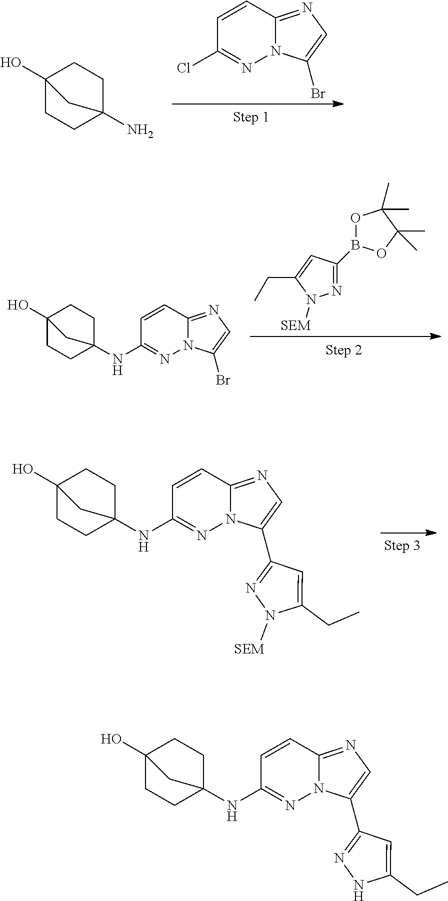

Step 1: 4-((3-bromoimidazo[1,2-b] pyridazin-6-yl)amino)bicyclo[2.2.1]heptan-1-ol was synthesized in accordance with the procedure described in Step 1 of the synthesis of Compound 5. LCMS (ESI positive ion) m/z: calculated: 325 (M+2).

Step 2: Preparation of 4-((3-(5-ethyl-1-((2-(trimethylsilyl)ethoxy)methyl)-1H-pyrazol-3-yl)imidazo[1,2-b]pyridazin-6-yl)amino)bicyclo[2.2.1]heptan-1-ol. To a stirred solution of 4-((3-bromoimidazo[1,2-b]pyridazin-6-yl)amino)bicyclo[2.2.1]heptan-1-ol (278 mg, 0.860 mmol) and 5-ethyl-3-(4,4,5,5-tetramethyl-1,3,2-dioxaborolan-2-yl)-1-((2-(trimethylsilyl)ethoxy)methyl)-1H-pyrazole (synthesized in accordance with the procedure described in Step 4 of the synthesis of Compound 6, 657 mg, 1.118 mmol) in 1,4-dioxane:water (5:1, 12 mL) was added Cs₂CO₃ (196 mg, 0.602 mmol) and Na₂CO₃ (137 mg, 1.290 mmol). The resulting reaction mixture was purged with nitrogen for 15 minutes. To this solution PdCl₂(dppf)-CH₂Cl₂ adduct (70.2 mg, 0.086 mmol) was added, and the reaction mixture was again purged with nitrogen for 15 minutes. The resulting mixture was stirred under microwave radiation at 120° C. for 2 h. After confirming the completion of reaction by LCMS, the reaction mixture was filtered through a pad of Celite, washed with excess EtOAc and concentrated under reduced pressure. The crude residue thus obtained was purified by flash column chromatography (silica gel, 230-400 mesh size) using 5% MeOH in DCM as an eluent to afford the title compound. LCMS (ESI positive ion) m/z: 469.2 (M+1).

Step 3: Preparation of 4-((3-(5-ethyl-1H-pyrazol-3-yl)imidazo[1,2-b]pyridazin-6-yl)amino)bicyclo[2.2.1]heptan-1-ol. To a stirred solution of 4-((3-(5-ethyl-1-((2-(trimethylsilyl)ethoxy)methyl)-1H-pyrazol-3-yl)imidazo[1,2-b]pyridazin-6-yl)amino)bicyclo[2.2.1]heptan-1-ol (300 mg, 0.416 mmol) in DCM (5 ml), TFA (0.032 ml, 0.416 mmol) was added to the reaction mixture at room temperature. The resulting mixture was stirred at room temperature for 18 h. After confirming the completion of reaction by LCMS, the reaction mixture was concentrated under reduced pressure. The crude residue was purified by reverse-phase, preparative HPLC (eluting with aqueous 0.1% TFA-acetonitrile) to afford the TFA salt of the title compound. LCMS (ESI positive ion) m/z: calculated: 339.2 (M+1). HPLC purity: 98.35%. ¹H-NMR (400 MHz, DMSO-d6): δ 8.19 (s, 1H), 7.97 (d, J=10.00 Hz, 1H), 7.78 (s, 1H), 7.10 (d, J=9.60 Hz, 1H), 6.90 (s, 1H), 2.73 (q, J=7.60 Hz, 2H), 2.18-2.21 (m, 2H), 1.83-1.92 (m, 4H), 1.72-1.82 (m, 2H), 1.60-1.70 (m, 2H), 1.29 (t, J=7.60 Hz, 3H).

### Example 2. RBC HotSpot™ Kinase Inhibition Assay

The RBC HotSpot™ Kinase inhibition assay was conducted in accordance with published protocol (see Anastassiadis T, et al., “Comprehensive assay of kinase catalytic activity reveals features of kinase inhibitor selectivity”, Nat Biotechnol. 2011 Oct. 30; 29(11):1039-45. doi: 10.1038/nbt.2017. PMID: 22037377; PMCID: PMC3230241), and further details of the method are summarized therein.

Reagents. Base Reaction Buffer: 20 mM Hepes (pH 7.5), 10 mM MgCl₂, 1 mM EGTA, 0.01% Brij35, 0.02 mg/ml BSA, 0.1 mM Na₃VO₄, 2 mM DTT, 1% DMSO. The following kinases were used in the assay at the indicated concentrations:

| | Kinase | Kinase | Kinase in |
|---|---|---|---|
| Kinase | Catalog No. | Provider | reaction (nM) |
| PIM1 | PV3503 | Invitrogen | 1 |
| PIM2 | PR6347B | Invitrogen | 0.4 |
| PIM3 | 14-738 | Millipore | 0.3 |

The following substrates were used in combination with the indicated kinases in the assay at the indicated concentration:

| | | | Substrate | Substrate in |
|---|---|---|---|---|
| Kinase | Substrate | Substrate Sequence | Provider | reaction (μM) |
| PIM1 | S6K/Rsk2 | KKRNRTLTK (SEQ ID NO: 1) | GenScript | 20 |
| | peptide 2 | | | |
| PIM2 | Pim2tide | RSRHSSYPAGT (SEQ ID NO: 2) | GenScript | 20 |
| PIM3 | Pim2tide | RSRHSSYPAGT (SEQ ID NO: 2) | GenScript | 20 |

No cofactors were added to any of the reactions.

### Reaction Procedure:

1. Prepared substrate in freshly prepared Reaction Buffer.
2. Delivered kinase into the substrate solution and gently mixed.
3. Delivered test compound in 100% DMSO into the kinase reaction mixture by Acoustic technology (Echo550; nanoliter range), incubated for 20 minutes at room temperature.
4. Delivered ³³P-ATP into the reaction mixture to initiate the reaction.
5. Incubated for 2 hours at room temperature.
6. Detected kinase activity by P81 filter-binding method.

The results of the RBC HotSpot™ kinase assay are set forth in Table 1, wherein “A” indicates an IC₅₀<10 nM, “B” indicates an IC₅₀ wherein 10 nM≤IC₅₀≤100 nM, and “C” indicates an IC₅₀>100 nM.

TABLE 1

| Cmpd. | | | PIM1 | PIM2 | PIM3 |
|---|---|---|---|---|---|
| No. | Structure | IUPAC Name | IC₅₀ | IC₅₀ | IC₅₀ |
| 1 | 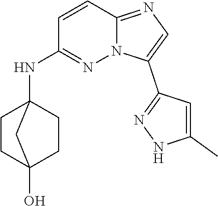 | 4-((3-(5-methyl-1H-pyrazol-3- yl)imidazo[1,2-b]pyridazin-6- yl)amino)bicyclo[2.2.1]heptan- 1-ol | C | C | C |
| 2 | 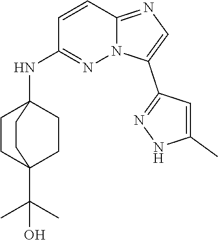 [SMILES: Cc1cc(-c2cnc3ccc(NC45CCC(C(C)(C)O)(CC4)CC5)nn23)n[nH]1] | 2-(4-((3-(5-methyl-1H-pyrazol- 3-yl)imidazo[1,2-b]pyridazin- 6- yl)amino)bicyclo[2.2.2]octan- 1-yl)propan-2-ol | A | C | A |
| 3 | 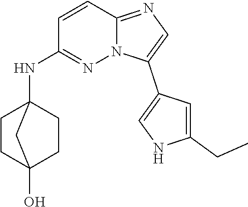 [SMILES: CCc1cc(-c2cnc3ccc(NC45CCC(O)(CC4)C5)nn23)c[nH]1] | 4-((3-(5-ethyl-1H-pyrrol-3- yl)imidazo[1,2-b]pyridazin-6- yl)amino)bicyclo[2.2.1]heptan- 1-ol | B | C | B |
| 4 | 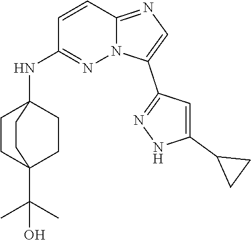 [SMILES: CC(C)(O)C12CCC(Nc3ccc4ncc(-c5cc(C6CC6)[nH]n5)n4n3)(CC1)CC2] | 2-(4-((3-(5-cyclopropyl-1H- pyrazol-3-yl)imidazo[1,2- b]pyridazin-6- yl)amino)bicyclo[2.2.2]octan- 1-yl)propan-2-ol | A | B | A |
| 5 | 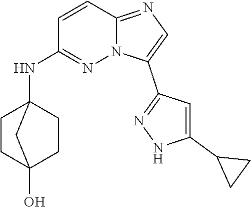 [SMILES: OC12CCC(Nc3ccc4ncc(-c5cc(C6CC6)[nH]n5)n4n3)(CC1)C2] | 4-((3-(5-cyclopropyl-1H- pyrazol-3-yl)imidazo[1,2- b]pyridazin-6- yl)amino)bicyclo[2.2.1]heptan- 1-ol | B | C | B |
| 6 | 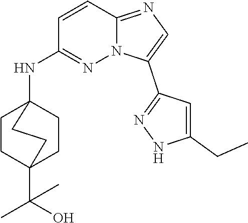 [SMILES: CCc1cc(-c2cnc3ccc(NC45CCC(C(C)(C)O)(CC4)CC5)nn23)n[nH]1] | 2-(4-((3-(5-ethyl-1H-pyrazol- 3-yl)imidazo[1,2-b]pyridazin- 6- yl)amino)bicyclo[2.2.2]octan- 1-yl)propan-2-ol | A | C | A |
| 7 | 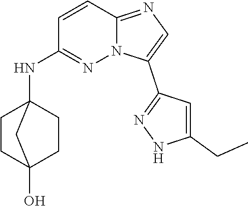 [SMILES: CCc1cc(-c2cnc3ccc(NC45CCC(O)(CC4)C5)nn23)n[nH]1] | 4-((3-(5-ethyl-1H-pyrazol-3- yl)imidazo[1,2-b]pyridazin-6- yl)amino)bicyclo[2.2.1]heptan- 1-ol | B | C | B |

### Example 3. In-Cell Western

PIM1 is a kinase, and has many downstream targets, including ribosomal protein S6, which controls translation and cell proliferation. PIM inhibition should result in a decrease of S6 phosphorylation without affecting total S6. To test the ability of Compounds 2 and 4 to decrease PIM activity, S6 phosphorylation was followed in A549 cells after incubation with Compound 2 or Compound 4 for 24 hours.

Procedure. Three thousand five hundred A549 cells (ATCC, CCL-185) were added per well of a 384-well plate in a total volume of twenty-five microliters, and cultured in RPMI medium (no phenol red) plus ten percent fetal calf serum. The next day, DMSO or test compound was added (90 nanoliters of test compound/DMSO per well) at 7 different concentrations starting at 10 micromolar (3.3-fold dilution). After 24 hours at 37° C. with 5% CO₂, cells were processed for in-cell Western (Li-Cor).

Briefly, medium was removed, and cells were immediately fixed with 4% formaldehyde in 1×PBS (50 microliters per well) for 20 minutes at room temperature with no shaking. Fixation solution was then discarded and wells were washed three times with 1×PBS containing 0.1% Triton® X-100. Cells were blocked by adding 50 microliters of Odyssey® Blocking Buffer (Li-Cor) to each well and incubating at room temperature (RT) with moderate shaking. Primary antibodies (Phospho-S6 (Ser235/236): Rabbit CST 2211S, (1/100 dilution) and total S6 Ribosomal Protein (54D2) Mouse mAb #2317, (1/100 dilution)) in 25 microliters of Odyssey® Blocking Buffer (Li-Cor) were then added to each well. The next day, primary antibodies were removed and wells were washed three times with 1×PBS containing 0.1% Tween-20. Secondary antibodies (IRDye® 800CW Goat anti-Mouse (1:800 dilution) and IRDye 680RD Goat anti-Rabbit (1:800 dilution)) prepared in Odyssey® Blocking Buffer (Li-Cor)+0.2% Tween-20 were added to each well, and the wells were incubated in the dark for an hour at RT with gentle shaking. The cells were washed three times with 1×PBS+0.1% Tween 20 with gentle shaking and imaged using the Odyssey Imaging system (Li-Cor). Signal for each channel was measured and correlated to the presence of total S6 (700 nm) or phospho-S6 (Ser235/236) (800 nm). The ratio between the phospho-S6 and total S6 (800 nm/700 nm) was calculated for each concentration.

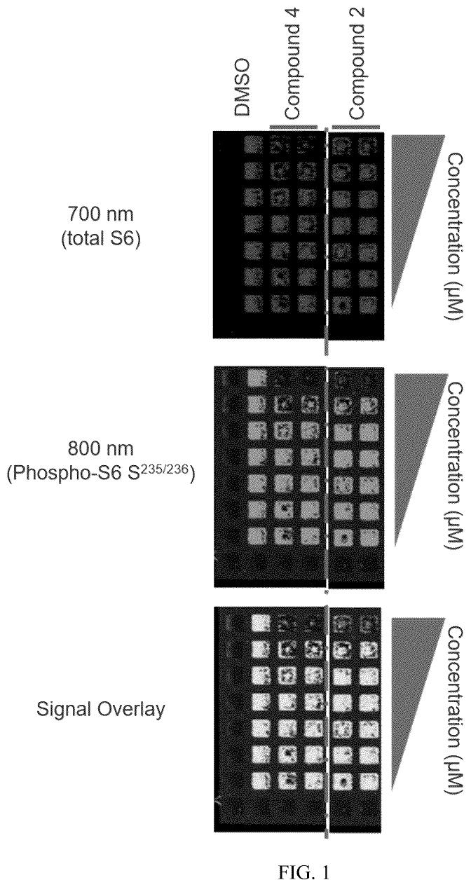

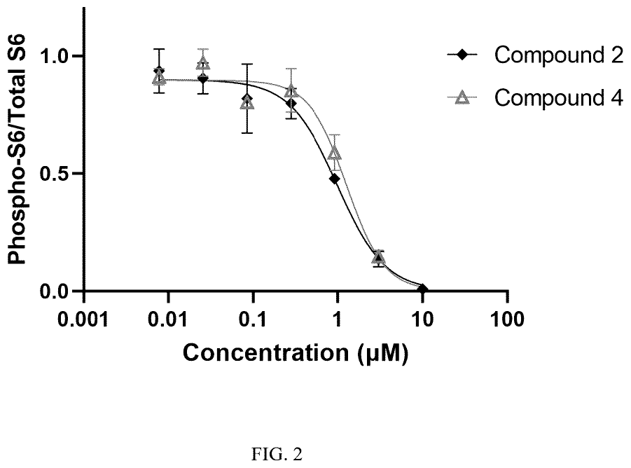

Results. FIG. 1 is a representative image of the in-cell Western blot, and shows expression of total S6 (red channel, 700 nm), phospho-S6 (Ser235/236) (green channel, 800 nm) and the overlay between the two channels. FIG. 2 shows the dose-response for Compounds 2 and 4. Both Compounds 2 and 4 strongly reduced S6 phosphorylation in this assay, with IC₅₀ values between 100 and 1,500 nM.

### Example 4. MilliPLEX Assay

Overview. PIM1 is a kinase, and has many downstream targets, including Bad (BCL2 associated agonist of cell death), a member of the BCL-2 family. BCL-2 family members are regulators of the programmed cell death pathways. Proapoptotic activity of Bad is regulated through its phosphorylation. Dephosphorylated Bad forms a heterodimer with Bcl-2 and Bcl-xL, inactivating them and thus allowing Bax/Bak-triggered apoptosis. Bad phosphorylation is thus anti-apoptotic, and Bad dephosphorylation is pro-apoptotic. PIM inhibition is expected to result in a decrease of phosphorylated Bad on serine 112 without affecting total Bad.

Procedure. Twelve thousand A549 cells (ATCC, CCL-185) were added per well of a 96-well plate (Nunc Edge, ThermoScientific, catalog number 167425) in a total volume of fifty microliters and cultured in RPMI medium (no phenol red) plus ten percent fetal calf serum. The next day, DMSO or test compound was added (90 nanoliters of drug/DMSO per well) at 7 different concentrations starting at 10 micromolar (3.3-fold dilution). After 24 hours at 37° C. with 5% CO₂, cells were being processed for protein expression using the 6-plex Bcl-2 Family Apoptosis Panel 1 Magnetic Bead Kit (Millipore, catalog number 48-682MAG)) and Luminex technology (MAGPIX).

Briefly, media was removed, and cells were washed with 100 μl cold 1×PBS. Wash buffer was removed and cells were lysed in 25 μl 1× lysis buffer (+ phosphatase/protease inhibitors). Lysis was performed at 4° C. on the stirring plate for 15 minutes. After 15 minutes, equal volume of assay buffer (25 l) was added to the cell lysate, mixed for ten seconds at 600 rpm and centrifuged for five minutes at 1000×g at 4° C. to remove the insoluble fraction. Beads were prepared following the manufacturer's recommendations (sonication for 20 seconds and vortexing for 30 seconds). Assay buffer was discarded and 25 μl of diluted cell lysate was added to each well containing 25 l of beads. Incubation was performed overnight while shaking at 700 rpm 4° C.

The next day, the supernatants were discarded, and the plate was washed twice with 100 μl assay buffer. Twenty-five microliters of the antibody solution (0.150 ml detection antibody plus 2.85 ml assay buffer) were added per well, and the plate was incubated for an hour at room temperature while shaking at 400 rpm. The antibody solution was then discarded, and 25 μl of SAPE solution (0.120 ml SAPE plus 2.8 ml assay buffer) were added per well. Plate was incubated for 15 minutes at room temperature while shaking at 400 rpm. Twenty-five microliters of amplification buffer were added to the SAPE solution, and plate was incubated for 15 minutes at room temperature while shaking at 400 rpm. The SAPE solution and amplification buffer were then discarded, 150 μl of assay buffer were added per well, and plate was incubated for 5 minutes at room temperature while shaking before reading it using the Luminex instrument (MAGPIX). The ratio between phospho-Bad (S112) and total Bad was calculated, and the IC₅₀ was determined.

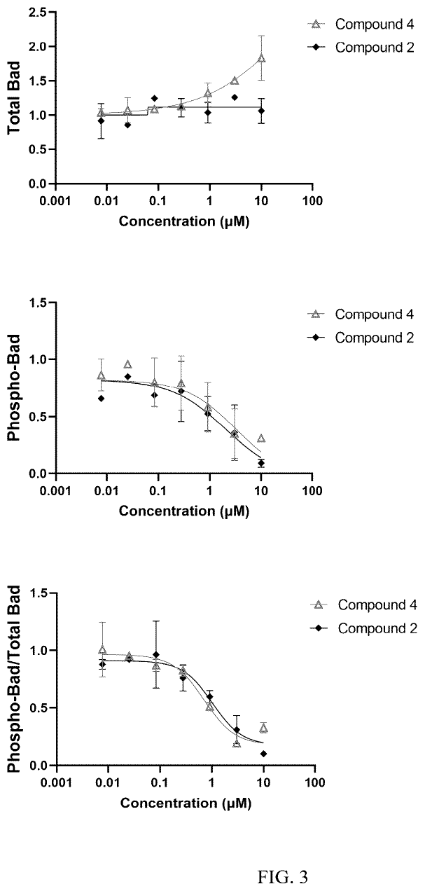

Results. FIG. 3 shows Bad expression in cells measured with the Luminex instrument, and the dose-response for Compounds 2 and 4. Bad phosphorylation was strongly reduced in the presence of Compounds 2 and 4, with IC₅₀ values of between 100 nM and 1 μM.

### Example 5. Apoptosis Assay

Overview. PIM1 is a kinase, and has many downstream targets including Bad (BCL2 associated agonist of cell death), a member of the BCL-2 family. BCL-2 family members are regulators of the programmed cell death pathways. Proapoptotic activity of Bad is regulated through its phosphorylation. Dephosphorylated Bad forms a heterodimer with Bcl-2 and Bcl-xL, inactivating them and thus allowing Bax/Bak-triggered apoptosis. Bad phosphorylation is thus anti-apoptotic, and Bad dephosphorylation is pro-apoptotic. PIM inhibition is expected to result in a decrease of phosphorylated Bad, and an increase in apoptosis.

Procedure. Three thousand five hundred A549 cells (ATCC, CCL-185) were added per well of a 384-well plate in a total volume of twenty-five microliters and cultured in RPMI medium (no phenol red)+plus ten percent fetal calf serum. The next day, DMSO or test compound was added (90 nanoliters of test compound/DMSO per well) at 7 different concentrations starting at 10 micromolar (3.3-fold dilution). After 24 hours at 37° C. with 5% CO₂, apoptosis was measured by using the Caspase 3/7 assay system (Promega, G8090). Briefly, equal volume of Caspase-Glo 3/7 reagent (25 microliters) was added per well and well contents were gently mixed for 30 seconds at 300 rpm. The cells were then incubated at room temperature for 30 minutes and luminescence was measured for each sample using a plate reading luminometer (Envision, PerkinElmer).

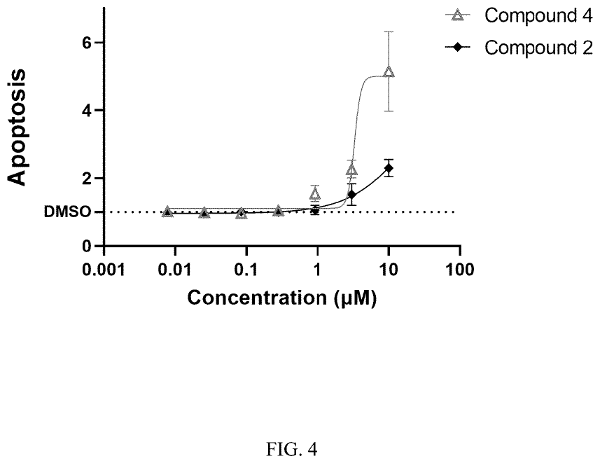

Results. FIG. 4 shows that Compounds 2 and 4 activated Caspase 3/7 with EC₅₀ values between 1 and 20 μM. This suggests that inhibition of Bad phosphorylation and therefore PIM activity is enhanced in presence of Compounds 2 and 4 in this assay.

### Example 6. Wound Healing

Overview. Epithelial to mesenchymal transition (EMT) is used to describe epithelial cells losing their cell to cell adhesion and polarity while gaining migratory and invasive properties. The process of EMT is required for normal embryonic development and wound healing, but has also been implicated in fibrosis. In humans, TGF-β signaling is the main driver of EMT, and inhibiting this pathway may be beneficial in various diseases such as fibrosis. A wound healing assay was used to study cell migration and cell-cell interaction, and probe the EMT properties of lung epithelial cells (A549) in presence of Compound 2 or Compound 4.

Procedure. A549 cells (ATCC, CCL-185) were seeded into the ImageLock plate (Essen Bioscience) at 2×10⁴ cells/well in 100 μL of complete growth media. Cells were incubated overnight at 37° C., 5% CO₂. The next day, cells were washed with 1×PBS and Compound 2, Compound 4, DMSO control (±TGF-β) or galunisertib (TGF-β inhibitor used as positive control) was added to the cells in a serum-free F12K media (0.2% DMSO final, 10 μM starting concentration, 3-fold dilution, 8-doses) in presence of 5 ng/ml TGF-β. Cells were incubated for 48 hours (until they reached confluency) at 37° C., 5% CO₂. Wounds were simultaneously created using a WoundMaker™ (Essen Bioscience). Debris and non-attached cells were removed by gently washing once with F12K.

Test compound or control compound and TGF-β were added to the cells in F12K+10% FBS as previously described. The ImageLock plate was put into IncuCyteS3 instrument installed inside a 5% CO₂ incubator. The plate was allowed to equilibrate in the IncuCyteS3 instrument for 15 minutes prior to scheduling the first scan and data recording. The plate was incubated for 72 hours at 37° C., and wound closure was recorded in real time with the IncuCyte S3 imaging system. Percentage of relative wound density was quantitatively calculated by the IncuCyte software.

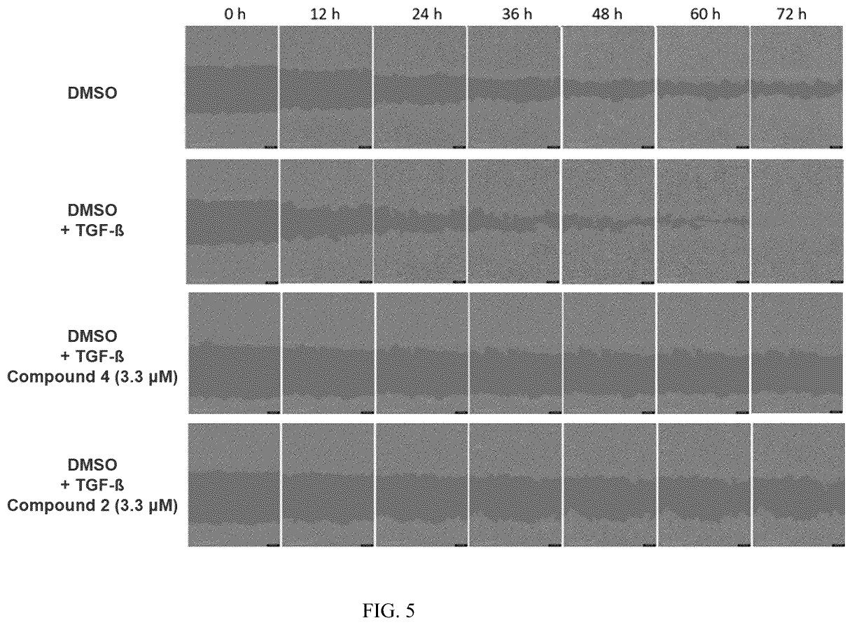

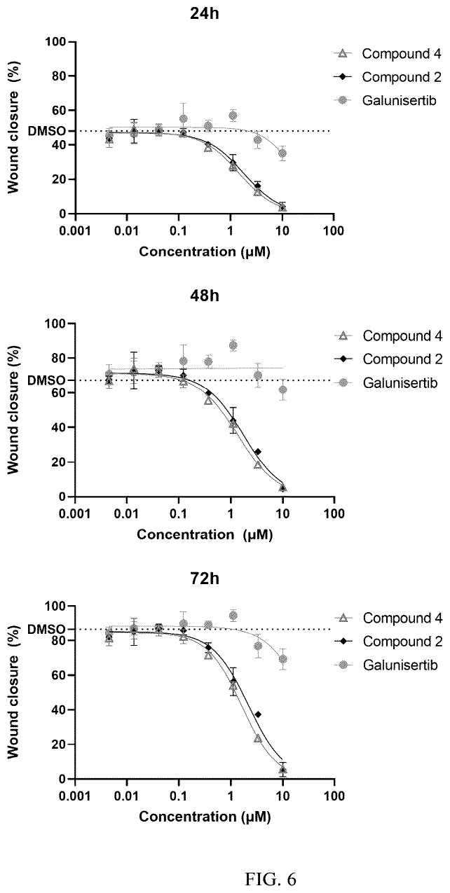

Results. FIG. 5 shows representative images of wound closure in scratched cells in the 96-well ImageLock plate over 24, 48 and 72 hours in presence of Compound 2, Compound 4 or DMSO (with or without TGF-β). FIG. 6 corresponds to the relative wound density measured from each condition described above. Table 2 shows the range of IC₅₀ values of Compound 2, Compound 4 and galunisertib in the wound healing assay at 24, 48 and 72 hours.

TABLE 2

| | IC50 (μM) | | |
|---|---|---|---|
| | 24 h | 48 h | 72 h |
| Compound 2 | 1-10 | 1-10 | 1-10 |
| Compound 4 | 1-10 | 1-10 | 1-10 |
| Galunisertib | >10 | | |

The data in FIGS. 5 and 6 and Table 2 show that after 72 hours, wound closure is completed in presence of the vehicle control with TGF-β. In contrast, migration is strongly inhibited in the presence of Compound 2 or 4. These results suggest that Compounds 2 and 4 could successfully be used in fibrosis.

### Example 7. Bleomycin-Induced Lung Fibrosis Study

Overview. The most common animal model of pulmonary fibrosis is the bleomycin-induced lung fibrosis model in rodents. This model is commonly used to investigate biology and potential therapies for fibrotic diseases affecting the lung, such as idiopathic pulmonary fibrosis (IPF). Bleomycin, a cytotoxic drug, is administered to mice or rats, and causes direct cell injury triggered by DNA strand breaks, which leads to an overproduction of reactive oxygen species causing inflammation, pulmonary toxicity, activation of fibroblasts and subsequent fibrosis. Fibrosis is hallmarked by aberrant activation of lung epithelial cells and accumulation of fibroblasts and myofibroblasts with excessive production of extracellular matrix such as collagen.

Hydroxyproline is a major component of protein collagen, and plays a key role in the stability of the collagen triple helix. Hydroxyproline levels are used as an indicator of the amount of collagen.

It has been shown that PIM1 silencing plays a critical role in accelerating the resolution of lung fibrosis by reversing the progress of EMT induced by bleomycin administration. To test the ability of Compounds 2 and 4 to reduce fibrosis in vivo, a bleomycin-induced lung fibrosis model in mice was utilized and end-points such as lung hydroxyproline levels, a surrogate marker of collagen and fibrosis, and histological scoring of fibrosis were performed.

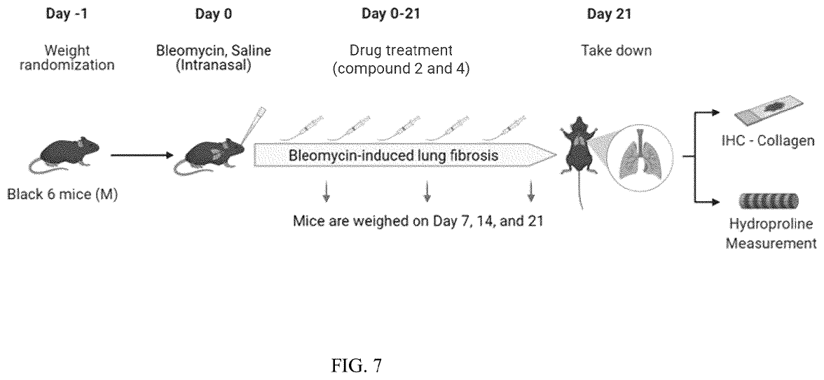

Procedure. FIG. 7 shows the overall study design. For this study, male C57BL/6J mice between 6 to 7 weeks old (purchased from Charles River) were weighed on the day prior to study start to establish a baseline. All treatment arms contained sixteen total animals except the sham/vehicle group, which contained 10 animals. All animals, except the sham/vehicle group were dosed intranasally with 4 international units per kilogram bleomycin in the morning of day 1 of the study. Bleomycin was prepared in a solution of 0.9 percent sodium chloride. All test compounds were prepared in 10 percent Tween 20. Starting in the evening on day one of the study, one dose of drug (ten or fifteen milligrams per kilogram of Compound 2 or fifteen or twenty milligrams per kilogram of Compound 4) was given, followed by twice a day daily dosing for the remainder of the twenty-one day study. At day twenty-one, the lungs were harvested and either snap frozen or fixed in paraformaldehyde. The snap frozen portion of the lung tissue was subjected to hydroxyproline quantification, which is used as a common biomarker of fibrosis. Additionally, lung tissue was fixed in paraformaldehyde to be processed for histological examination following hematoxylin and eosin (H&E) and Masson's trichrome stainings. The histological sections were scored using a modified Ashcroft scale, the number of mononuclear cell aggregates and alveolar mixed infiltrates as well as the levels of collagen. The mice were weighed on day seven, fourteen, and twenty-one to monitor weight changes, and mortality and clinical observations were recorded daily. The following modified Ashcroft scale was used:
Grade 0=Normal lung
Grade 1=Minimally detectable thickening of alveolar walls
Grade 2=Mild thickening of alveolar walls
Grade 3=Moderate contiguous thickening of walls with fibrous nodules
Grade 4=Thickened septae and confluent fibrotic masses totaling less than 10% of the microscopic field
Grade 5=Increased fibrosis with definite damage to lung structure and formation of fibrous bands or small fibrous masses between 10-50% of the microscopic field
Grade 6=Large contiguous fibrotic masses consolidating more than 50% of the microscopic field
Grade 7=Severe distortion of structure and large fibrous areas
Grade 8=Total fibrous obliteration of lung within the microscopic field

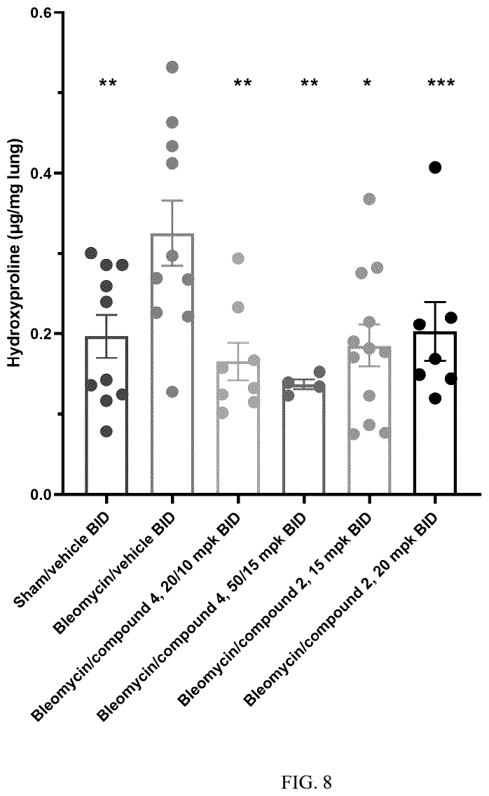

Results. FIG. 8 shows the amount of hydroxyproline (micrograms of hydroxyproline per milligram of lung tissue) measured from a portion of the lung tissue harvested from each mouse remaining on day twenty-one of the study. Hydroxyproline levels were reduced in all treatment groups, to levels similar to the sham/vehicle group. In fact, using the Dunnett's multiple comparisons test, all groups showed a statistically significant difference in the amount of hydroxyproline compared to the bleomycin/vehicle group. The greatest and most statistically significant reduction of hydroxyproline was found in the arm of animals receiving 15 milligrams per kilogram of Compound 4 twice a day.

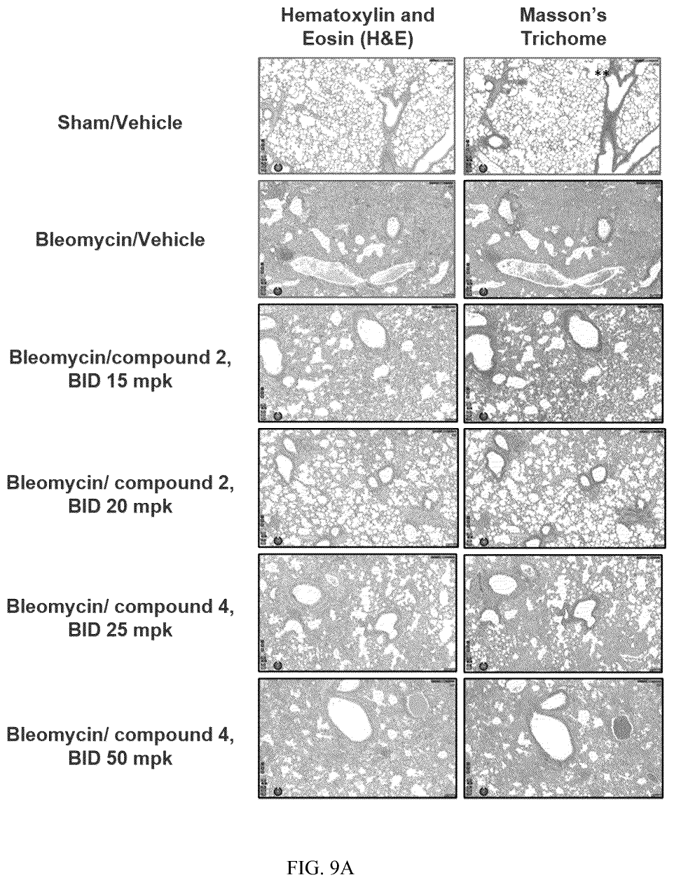

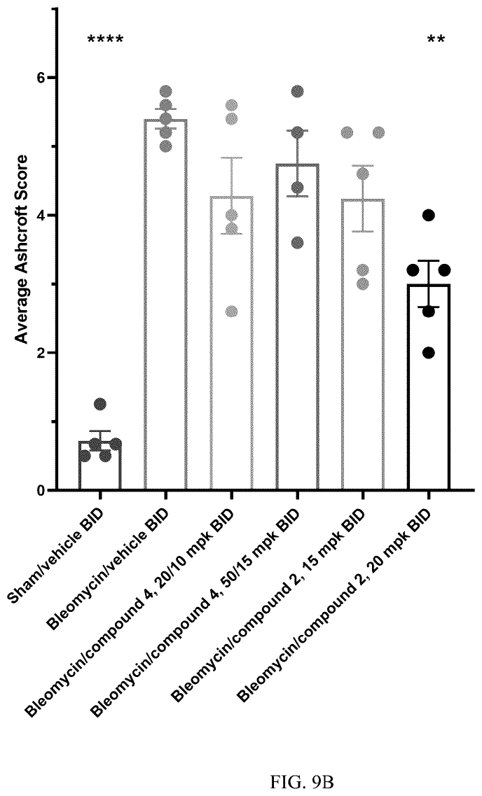

FIG. 9A shows representative histological images of the lung tissues from each group stained with Masson's trichrome and H&E. FIG. 9B corresponds to the analysis using the Ashcroft score and other cell scorings of five randomly chosen animals from each treatment group. Every score was determined by a pathologist using the modified Ashcroft scale set forth above. The Ashcroft score is used to indicate the fibrosis severity and lung tissue structure changes due to the fibrosis. At least 2 out of the 5 total animals in each treatment group had a reduction of the Ashcroft score compared to the average Ashcroft score in the vehicle group. The Ashcroft score was significantly reduced in the arm of animals receiving 20 milligrams per kilogram of Compound 2 twice a day.

In summary, PIM1 inhibition by Compound 2 may be a useful therapeutic strategy for pulmonary progressive fibrotic disorders.

### Example 8. Psoriasis Study

Overview. Cytokines produced by T cells are predominant mediators of skin pathology in psoriasis, a chronic inflammatory skin disease. Accumulating evidence suggests that interleukin (IL)-23 and the subsequent T helper 17 (Th17) cell response it promotes are central factors in the pathogenesis of this disease.

It has been shown that PIM1 is overexpressed in patients with psoriasis compared to healthy individuals. Moreover, studies involving PIM1 knockout mice and SuperGen's first generation PIM inhibitor (SGI-1776) demonstrated partial reduction in vascularization and epidermal thickening in mouse psoriasis models.

The IL-23-induced hyperplasia model involves the injection of IL-23 into the ear of mice, and is dependent on IL-22 for the development of dermal inflammation and acanthosis. This model is commonly used to investigate pathways involved in psoriasis as well as potential therapies.

IL-17A and IL-22 are found in excess in skin affected by psoriasis. IL-17A is a major effector cytokine in the pathogenesis of psoriatic disease. IL-22 mediates keratinocyte proliferation and epidermal hyperplasia, inhibits terminal differentiation of keratinocytes, and induces the production of antimicrobial proteins.

To test the ability of Compounds 2 and 4 to reduce psoriasis in vivo, an IL-23 induced hyperplasia mouse model was utilized, and analyses of end-points, such as IL-17A and IL-22 levels, and histological measure of epidermal thickness of the ear tissue, were performed.

Procedure. Female C57BL/6J mice between 9 to 13 weeks old (purchased from Taconic Biosciences) were weighed on the day prior to study start to establish a baseline. All treatment arms contained ten animals. All animals, except the sham/vehicle group received 0.4 μg of recombinant mouse IL-23 (rmIL-23) in a volume of 20 μL intradermally into one ear. Mice in group 1 (sham/vehicle) received 20 μL PBS. A total of 4 injections per ear was performed while the second ear was not injected nor analyzed. All drugs were prepared in 0.5 percent methylcellulose and 2 percent Tween 80. Starting on day zero of the study (0.5 to 4 hours before rmIL-23 injection), two doses of test compound (100 or 150 milligrams per kilogram of Compound 4, or 50 or 75 milligrams per kilogram of Compound 2) were given. All dosing was at the same time (+/−1 hour) each day with no less than 10 hours and no more than 14 hours between doses.

On days zero (before rmIL-23 injection), three, and four (approximately 24 hours after the final hyperplasia induction injection), thickness of ears was measured using a caliper. Measurements were performed blind, by a person unaware of group or previous measurements.

On day four, approximately 24 hours after the final hyperplasia induction injection, all mice were euthanized, and treated ears collected and weighed. Half of each ear was weighed and frozen for cytokine analysis (IL-17A and IL-22 concentration was measured using the Luminex technology by ELISA). The other half of each ear were fixed in 10 percent buffered formalin following H&E staining for histological analysis.

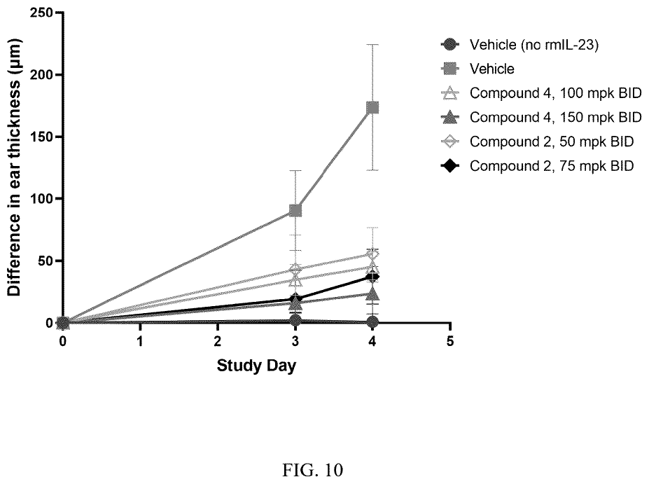

Results. Gross ear thickness, as measured in live mice with a caliper, is greater in rmIL-23-injected mice when compared with saline-injected mice (FIG. 10). Treatment with Compound 2 or Compound 4 significantly reduced gross ear thickness compared to the rmIL-23/vehicle control group. The greater effect was observed for the group treated with Compound 4 at 150 milligrams per kilogram twice a day.

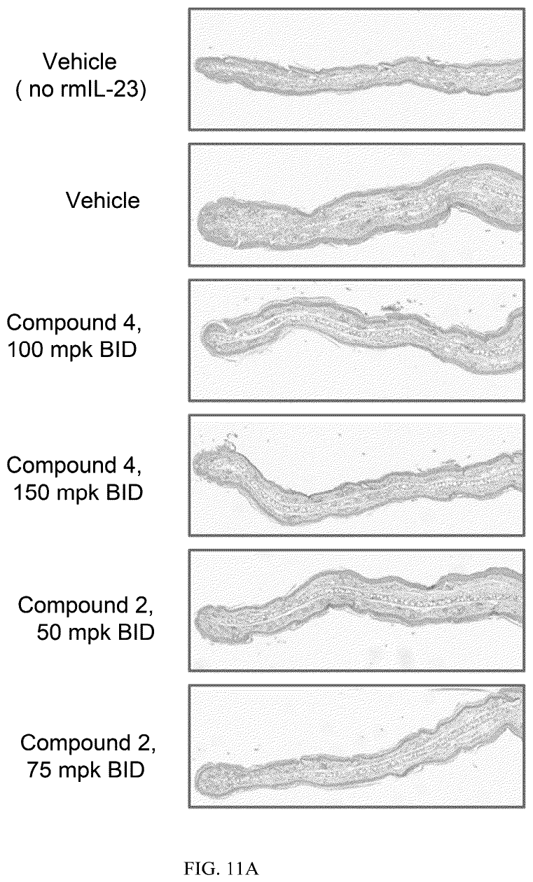

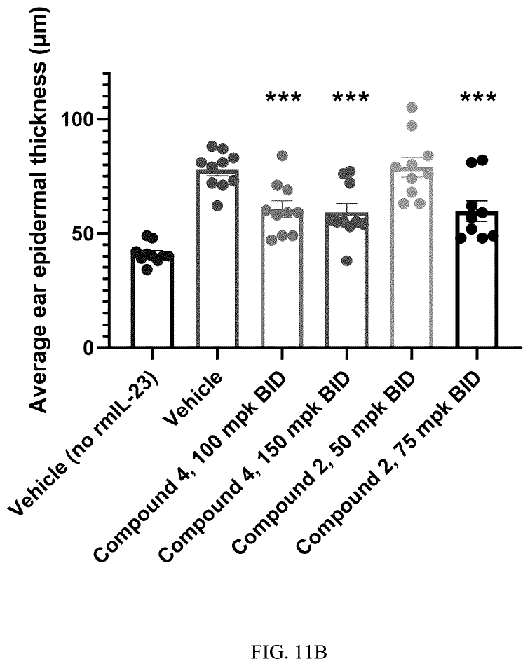

H&E staining of ear sections shows that rmIL-23-injected mice had a thicker epidermal layer compared to saline-injected mice (FIG. 11). Quantification of histology analysis revealed that dosing with Compound 4 (100 or 150 milligrams per kilogram twice a day) or Compound 2 (75 milligrams per kilogram twice a day) significantly reduced the thickness of the ear epidermal tissue compared to the group treated with rmIL-23/vehicle.

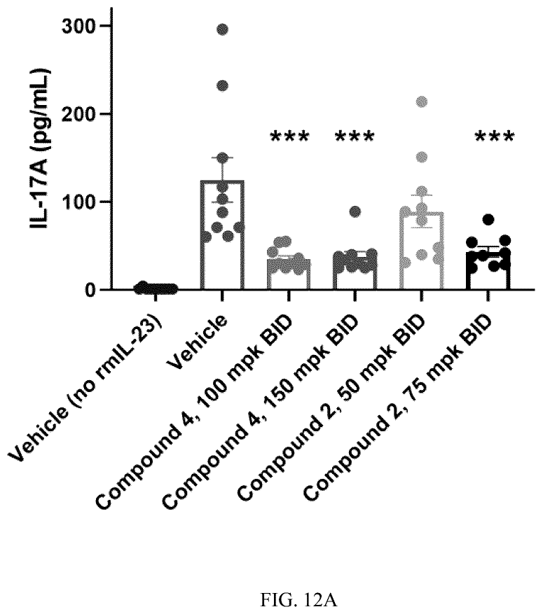

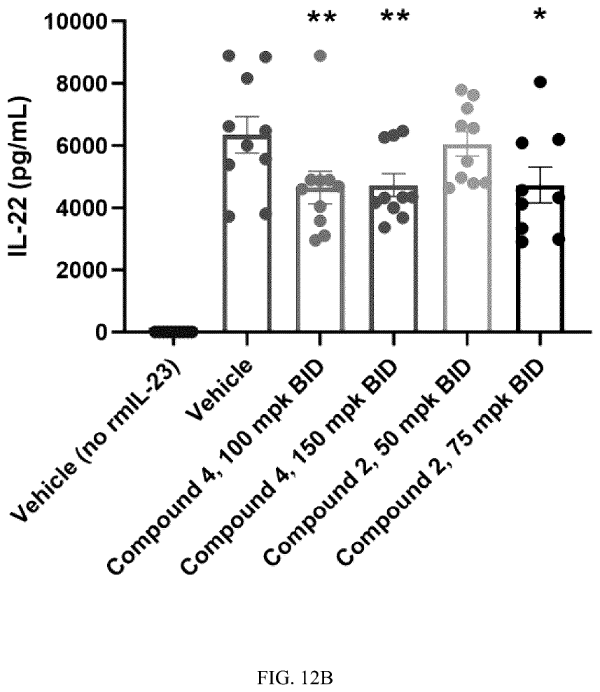

IL-17A and IL-22 concentrations from frozen ear tissue, as determined on the final day of the study, were higher in rmIL-23-injected mice when compared with saline-injected mice (FIG. 12). Treatment with Compound 2 (75 milligrams per kilogram twice a day) or with Compound 4 (100 or 150 milligrams per kilogram twice a day) strongly inhibited IL-17A production and reduced IL-22 synthesis compared to the rmIL-23/vehicle control group, suggesting that PIM1 inhibition by Compound 2 or 4 may be a useful therapeutic strategy for psoriasis.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A compound of the following structural formula: 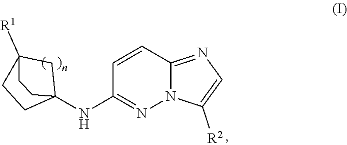
or a pharmaceutically acceptable salt thereof, wherein:
R¹ is —H, —OH, (C₁-C₃)alkyl or (C₁-C₃)hydroxyalkyl;
R² is (C₅-C₆)heteroaryl optionally substituted with one or more R²⁰;
each R²⁰ is independently —OH, halo, —CN, (C₁-C₃)alkyl, (C₁-C₃)haloalkyl, (C₃)carbocyclyl, —NH₂, —N(H)S(O)₂(C₁-C₃)alkyl or —N(CH₃)₂; and
n is 1 or 2.

2. The compound of claim 1, wherein R¹ is —OH or (C₁-C₃)hydroxyalkyl;
optionally wherein: R¹ is —OH or —C(CH₃)₂OH.

3. The compound of claim 1 or claim 2, wherein R² is (C₅)heteroaryl optionally substituted with one or more R²⁰;
optionally wherein: R² is pyrazolyl or pyrrolyl optionally substituted with one or more R²⁰.

4. The compound of any one of claims 1-3, wherein R² is substituted with one R²⁰.

5. The compound of any one of claims 1-4, wherein each R²⁰ is independently halo, —CN, (C₁-C₃)alkyl, (C₁-C₃)haloalkyl or cyclopropyl;
optionally wherein:
each R²⁰ is independently (C₁-C₃)alkyl or cyclopropyl; or
each R²⁰ is independently methyl, ethyl or cyclopropyl.

6. The compound of any one of claims 1-5, wherein n is 1.

7. The compound of any one of claims 1-5, wherein n is 2.

8. The compound of any one of claims 1, 2, 6, or 7, of the following structural formula: 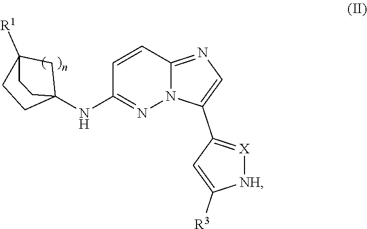
or a pharmaceutically acceptable salt thereof, wherein:
X is N or C(H); and
R³ is —H, —OH, halo, —CN, (C₁-C₃)alkyl, (C₁-C₃)haloalkyl, (C₃)carbocyclyl, —NH₂, —N(H)S(O)₂CH₃ or N(CH₃)₂.

9. The compound of claim 8, wherein X is N.

10. The compound of claim 8, wherein X is C(H).

11. The compound of any one of claims 8-10, wherein R³ is —H, halo, —CN, (C₁-C₃)alkyl, (C₁-C₃)haloalkyl or cyclopropyl;
optionally wherein:
R³ is (C₁-C₃)alkyl or cyclopropyl; or
R³ is methyl, ethyl or cyclopropyl.

12. The compound of claim 1, wherein the compound has the formula: 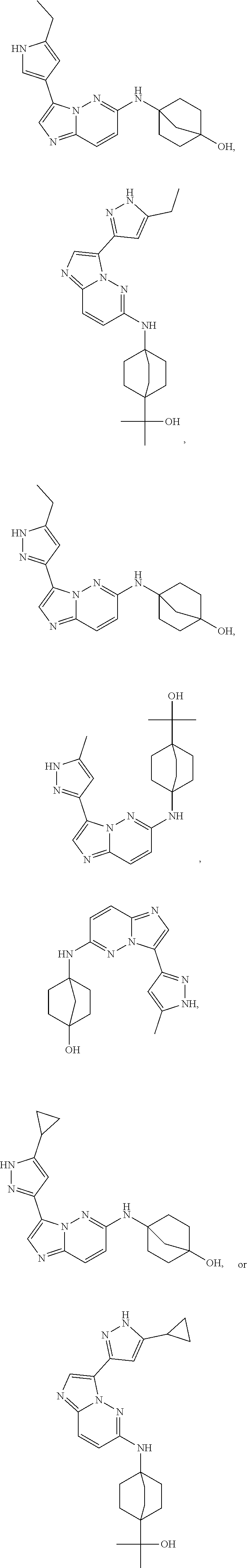
or a pharmaceutically acceptable salt thereof.

13. A pharmaceutical composition comprising a compound of any one of claims 1-12, or a pharmaceutically acceptable salt thereof, and a pharmaceutically acceptable carrier.

14. A pharmaceutical combination comprising a compound of any one of claims 1-12, or a pharmaceutically acceptable salt thereof, or a pharmaceutical composition of claim 13, and one or more additional therapeutic agents.

15. A method of inhibiting a proviral integration site for Moloney murine leukemia virus (PIM) kinase in a cell, comprising contacting the cell with a compound of any one of claims 1-12, or a pharmaceutically acceptable salt thereof, a pharmaceutical composition of claim 13 or a pharmaceutical combination of claim 14.

16. A method of inhibiting a PIM kinase in a subject, comprising administering to the subject a compound of any one of claims 1-12, or a pharmaceutically acceptable salt thereof, a pharmaceutical composition of claim 13 or a pharmaceutical combination of claim 14 in an amount effective to inhibit the PIM kinase in the subject.

17. A method of treating a disease, disorder or condition associated with a PIM kinase in a subject in need thereof, comprising administering to the subject a therapeutically effective amount of a compound of any one of claims 1-12, or a pharmaceutically acceptable salt thereof, a pharmaceutical composition of claim 13 or a pharmaceutical combination of claim 14.

18. A method of treating a fibrotic disease, disorder or condition in a subject in need thereof, comprising administering to the subject a therapeutically effective amount of a compound of any one of claims 1-12, or a pharmaceutically acceptable salt thereof, a pharmaceutical composition of claim 13 or a pharmaceutical combination of claim 14, wherein the fibrotic disease, disorder or condition is present in the absence of a fibrotic cancer;
optionally wherein:
the fibrotic disease, disorder or condition is lung fibrosis, liver fibrosis, cardiac disease, cardiac fibrosis or restenosis, vascular fibrosis, kidney fibrosis, skin fibrosis, gastrointestinal fibrosis, bone marrow fibrosis, athrofibrosis, Dupuytren's contracture, mediastinal fibrosis, Peyronie's disease, retroperitoneal fibrosis, systemic sclerosis, autoimmune hepatitis, nonalcoholic steatohepatitis, cystic fibrosis, beta-thalassemia, actinic keratosis, hypertension, chronic kidney disease, Chagas' heart disease, dry eye, ulcer, corneal fibrosis, wet age-related macular degeneration, chronic wound, or psoriasis; or
the fibrotic disease, disorder or condition is lung fibrosis, optionally wherein the lung fibrosis is idiopathic pulmonary fibrosis; or
the fibrotic disease, disorder or condition is liver fibrosis; or
the fibrotic disease, disorder or condition is cardiac fibrosis or restenosis.

19. A method of treating inflammation in a subject in need thereof, comprising administering to the subject a therapeutically effective amount of a compound of any one of claims 1-12, or a pharmaceutically acceptable salt thereof, a pharmaceutical composition of claim 13 or a pharmaceutical combination of claim 14.

20. A method of treating an inflammatory disease, disorder or condition in a subject in need thereof, comprising administering to the subject a therapeutically effective amount of a compound of any one of claims 1-12, or a pharmaceutically acceptable salt thereof, a pharmaceutical composition of claim 13 or a pharmaceutical combination of claim 14;
optionally wherein:
the inflammatory disease, disorder or condition is non-alcoholic fatty liver disease (NAFLD), alcoholic steatohepatitis (ASH), non-alcoholic steatohepatitis (NASH), primary biliary cholangitis (PBC), primary sclerosing cholangitis, autoimmune hepatitis, skin inflammation or psoriasis; or
the inflammatory disease, disorder or condition is an autoimmune disease, disorder or condition, optionally wherein the autoimmune disease, disorder or condition is osteoarthritis, rheumatoid arthritis, pain, inflammatory bowel disease, a respiratory disorder or a skin disorder.

21. The method of any one of claims 16-20, further comprising administering to the subject one or more additional therapeutic agents.
